(19) United States
(12) Patent Application Publication
(10) Pub. No.: US 2011/0166194 A1
(43) Pub. Date: Jul. 7, 2011

(54) Asenapine Prodrugs

(75) Inventors: Laura Cook Blumberg, Lincoln, MA (US); Örn Almarsson, Shrewsbury, MA (US)

(73) Assignee: Alkermes, Inc., Waltham, MA (US)

(21) Appl. No.: 12/978,298

(22) Filed: Dec. 23, 2010

Related U.S. Application Data
(60) Provisional application No. 61/293,171, filed on Jan. 7, 2010.
(60) Provisional application No. 61/293,163, filed on Jan. 7, 2010.

(51) Int. Cl. A61K 31/407; C07D 491/04; A61P 25/18

(52) U.S. Cl. 514/410; 548/421

(57) ABSTRACT

Compounds of Formula I and their use for the treatment of neurological and psychiatric disorders including schizophrenia and manic or mixed episodes associated with bipolar I disorder with or without psychotic features is disclosed:

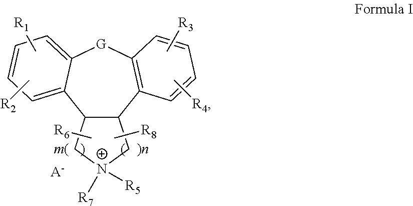

wherein R₁-R₈, G, N and A⁻ are as defined in the written description.

### RELATED APPLICATION(S)

This application claims the benefit of U.S. Provisional Application Nos. 61/293,171 and 61/293,163, both filed on Jan. 7, 2010. The entire teachings of the above application(s) are incorporated herein by reference.

### BACKGROUND OF THE INVENTION

Asenapine is one of several heterocyclic drugs approved by the U.S. Food and Drug Administration for the acute treatment of schizophrenia in adults, and acute treatment of manic or mixed episodes associated with bipolar I disorder, with or without psychotic features in adults. Asenapine has demonstrated efficacy for improving the positive (delusions and hallucinations) and negative (emotional withdrawal, apathy, avolition, and cognitive dysfunction) symptoms of schizophrenia, while showing limited extrapyramidal, antimuscarinic, and metabolic adverse affects. Asenapine binds to multiple receptors for neurotransmitters, with higher affinity to a variety of serotonergic (5-HT_{2A}, 5-HT_{2C}, 5-HT₆, 5-HT_{S}), noradrenergic (α_{2A}, α_{2B}, α_{2C}) and dopaminergic (D₃, D₄) receptors than for D₂ receptors, and with minimal affinity for muscarinic receptors. The chemical structure of asenapine is given below.

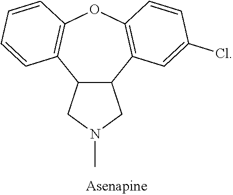

Other examples of heterocyclic derivatives that are useful for the treatment of schizophrenia are discussed in U.S. Pat. No. 5,350,747, U.S. Pat. No. 5,006,528, U.S. Pat. No. 7,160,888, and in U.S. Pat. No. 6,127,357. Heterocyclic derivatives that have been stated to be useful as antipsychotic agents are discussed in WO 93/04684 and European patent application EP 402644.

Drug delivery systems are often critical for the safe and effective administration of a biologically active agent. Perhaps the importance of these systems is best realized when drug bioavailability, patient compliance, and consistent dosing are taken under consideration. For instance, reducing the dosing requirement for a drug from four-times-a-day to a single dose per day, or to once a week or even less frequently would have significant value in terms of ensuring patient compliance.

In an attempt to address the need for improved bioavailability several drug release modulation technologies have been developed. Enteric coatings have been used as a protector of pharmaceuticals in the stomach and microencapsulating active agents using protenoid microspheres, liposomes or polysaccharides have been effective in abating enzyme degradation of the active agent. Enzyme inhibiting adjuvants have also been used to prevent enzyme degradation.

While microencapsulation and enteric coating technologies impart enhanced stability and time-release properties to active agent substances, these technologies suffer from several shortcomings. Incorporation of the active agent is often dependent on diffusion into the microencapsulating matrix, which may not be quantitative and may complicate dosage reproducibility. In addition, encapsulated drugs rely on diffusion out of the matrix or degradation of the matrix, which is highly dependent on the water solubility of the active agent. Conversely, water-soluble microspheres, based on their ability to swell rapidly and disintegrate in aqueous medium, may release the active agent in bursts with little active agent available for sustained release. Additionally, there is a need for an active agent delivery system that is able to deliver certain active agents which have been heretofore not formulated or difficult to formulate in a sustained release formulation, and which is convenient for patient dosing.

Tertiary amine-containing drugs have been derivatized to form compounds that enhance solubility of the parent tertiary amine-containing drug and improve targeting of the drug in the body and ultimately release the parent drug in its original form for further pharmacological action. These compounds, derivatized from tertiary amine-containing parent drugs, are referred to in the prior art as “delivery systems”, “transient delivery systems”, “prodrugs”, or promoieties, and comprise quaternary ammonium salts of parent drug compounds that are labile to enzymatic and/or chemical cleavage in vivo. However, the derivatives, promoieties and prodrugs of parent tertiary amine-containing drugs of the prior art are concerned with increasing solubility of these drugs, protecting labile moieties on the parent drugs and achieving rapid release of the parent drug from the prodrug moiety with minimal toxicity. Thus far there have been no prodrugs of tertiary amine-containing drugs that provide sustained release or zero-order kinetics by decreasing the solubility of the parent drug.

### SUMMARY OF THE INVENTION

The instant application relates to compounds of Formula I and their use for the treatment of neurological and psychiatric disorders including schizophrenia, mania, and bipolar disease. In particular the instant application relates to quaternary ammonium salt compounds of formula I:

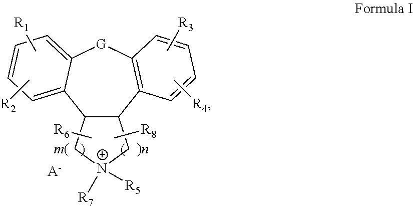

or its geometric isomers, enantiomers, diastereomers, racemates, pharmaceutically acceptable salts and solvates thereof;
wherein each m and n is independently selected from 0, 1, 2 or 3;
each R₁, R₂, R₃, R₄, R₆, R₇, and R₈ is independently selected from absent, hydrogen, hydroxy, halogen, —OR₁₀, —SR₁₀, —N(R₁₀)(R₁₁)—, optionally substituted aliphatic, optionally substituted aryl or optionally substituted heterocyclyl;
wherein each R₁₀ and R₁₁ are independently hydrogen, halogen, aliphatic, substituted aliphatic, aryl or substituted aryl;
   alternatively, two of R₁, R₂, R₃ and R₄ together form an optionally substituted ring;
   R₅ is selected from —C(R₁₀)(R₁₁)—OR₁₂, —C(R₁₀)(R₁₁)—OC(O)OR₂₁, —C(R₁₀)(R₁₁)—OC(O)R₂₁, —C(R₁₀)(R₁₁)—OC(O)NR₁₂R₂₁, —C(R₁₀)(R₁₁)—OPO₃²⁻MY, —C(R₁₀)(R₁₁)—OP(O)(O⁻M)(OR₂₁), —C(R₁₀)(R₁₁)—OP(O)(OR₂₁)(O R₂₂);
wherein each R₁₂ is independently hydrogen, halogen, aliphatic, substituted aliphatic, aryl or substituted aryl;
each R₂₁ and R₂₂ is independently hydrogen, aliphatic, substituted aliphatic, aryl or substituted aryl;
   G is selected from —O—, —S—, —NR₁₀, or —C(R₁₀)(R₁₁)—;
   Y and M are the same or different and each is a monovalent cation;

or M and Y together is a divalent cation; and,

A- is a pharmaceutically acceptable counterion.

The quaternary ammonium compounds of the invention incorporate a labile bio-activatable prodrug moiety which is cleaved in vivo to produce a bioactive compound such as asenapine. The addition of the prodrug moiety allows modification of the physical properties of the parent drug providing extended-release formulations. In addition, in embodiments in which the prodrug moiety comprises a phosphonate group, modification of the phosphonate group, through esterification with lipophilic groups, will modulate the solubility of the prodrugs. The physical chemical and solubility properties of these derivatives can be further modulated by the choice of counterion A⁻ (for example Cl⁻, Br⁻, I⁻, CH₃CO₂⁻, or other organic anions).

In one preferred embodiment, the prodrug compound of formula I further comprises a biocompatible delivery system for delivering the prodrug wherein the system is capable of minimizing accelerated hydrolytic cleavage of the prodrug by minimizing exposure of the prodrug to water. Preferred delivery systems include biocompatible polymeric matrix delivery systems capable of minimizing diffusion of water into the matrix.

### BRIEF DESCRIPTION OF THE DRAWINGS

FIG. 1: Solution stability of asenapine N-methyleneoxy octanoate as a function of pH.

FIG. 2: Solution stability of asenapine N-methyleneoxy pivalate as a function of pH.

FIG. 3: Pharmacokinetic analysis of asenapine:maleate, asenapine N-methyleneoxy palmitate chloride, and asenapine N-methyleneoxy (α,α-)dimethyl-butyrate iodide.

### DETAILED DESCRIPTION OF THE INVENTION

Currently asenapine is not available in an extended release formulation. The prodrug compounds of the present invention having the general structure of Formula I provide sustained or extended release to a parent compound such as asenapine. The terms “sustained release”, “sustained delivery” and “extended release” are used interchangeably herein to indicate that the prodrugs of the invention provide release of the parent drug by any mechanism including slow first-order kinetics of absorption or zero-order kinetics of absorption, such that the parent drug which is released from the prodrug provides a longer duration of action than the duration of action of the parent drug when administered alone (i.e. not as a prodrug of the invention). In accordance with the invention, “sustained release” of the prodrugs of the invention may include other pharmacokinetic indices such as a lower maximum concentration (Cmax) of parent drug in the blood and/or an extended period of time for the parent drug to reach maximum concentration in the blood (Tmax) as compared to the Cmax and Tmax when the parent drug is administered alone. Sustained release may also decrease concentration fluctuations in the body, as indicated by plasma concentration-time profiles.

Without being limited to any theory, the mechanism for sustained release of the prodrugs of the invention may be due to several factors including, but not limited to, the decreased solubility of the prodrug as compared to the parent drug at a reference pH such as the pH wherein the parent drug (not in prodrug form) would generally be fully protonated (e.g. around a pH 5.0). Such lower solubility of the prodrug at the reference pH may result in more gradual dissolution and slower release of the parent drug by the action of serum enzymes or chemical hydrolysis. In addition or alternatively, the mechanism of sustained release may be the result of the pH-independent solubility properties characteristic of the prodrugs of the invention that result in controlled and sustained release of the parent drug from the prodrug.

It has also been discovered that the reduction or elimination in pH-dependence of solubility of the prodrug compounds of the invention can be exploited in novel ways. The reduced solubility of the prodrug of the invention is maintained even if the prodrug is administered into an environment of fluctuating pH such as would be encountered in the stomach or at the site of injection. The pH independence of aqueous solubility for a prodrug of the invention also reduces or eliminates the problem of “dose dumping” (i.e. the undesirable rapid release of active agent upon administration of a sustained release formulation) which may occur in a sustained release formulation that is susceptible to changes in solubility in response to fluctuations in pH of the surrounding environment.

In one embodiment, the prodrugs of the present invention provide an extended period during which an active agent is absorbed thereby providing a longer duration of action per dose than is currently expected. This leads to an overall improvement of dosing parameters and the potential for less frequent dosing or improved pharmacokinetics for the duration of the currently prescribed dosing intervals.

“Effective amounts” or a “therapeutically effective amount” of a prodrug of the invention is based on that amount of the parent drug which is deemed to provide clinically beneficial therapy to the patient. In one embodiment, the prodrug of the invention provides an effective amount for a longer period of time per dose than that of the parent drug per the same dose when delivered alone.

In one embodiment, the prodrugs of the invention provide a lower Cmax of the parent drug as compared to the parent drug when administered alone. A lower Cmax means that dose dumping is minimized or avoided and that the side effects of the drug are also generally reduced or eliminated.

In one embodiment, a prodrug compound of the invention is less soluble in an aqueous solution such as a phosphate buffer at a reference pH than the parent drug. As used herein the term “reference pH” refers to the pH at which the aqueous solubility of a prodrug of the invention is compared to the aqueous solubility of the parent drug (not in prodrug form). Generally the reference pH is the pH at which the parent drug is fully protonated. Typically, the reference pH is about 5 and is preferably in the range of 4-6 and is more preferably in the range of about 4 to about 7. In another embodiment, the aqueous solubility of a prodrug compound of the invention at the reference pH is at least an order of magnitude lower than that of the aqueous solubility of the parent drug.

In one embodiment, a prodrug compound of the invention has an aqueous solubility in a phosphate buffer, at room temperature of less than about 0.1 mg/ml, preferably less than about 0.01 mg/mL, preferably less than about 0.001 mg/mL and even more preferably less than about 0.00001 mg/ml at a pH of about 6.

In one embodiment, a compound of the invention provides sustained delivery of the parent drug over hours, days, weeks or months when administered parenterally to a subject. For example, the compounds can provide sustained delivery of the parent drug for up to 7, 15, 30, 60, 75 or 90 days or longer. Without being bound by theory, it is believed that the compounds of the invention form an insoluble depot upon parenteral administration, for example subcutaneous, intramuscular or intraperitoneal injection.

In another embodiment, the prodrug of the invention provides sustained delivery of the parent drug when delivered orally. The prodrugs of the invention are generally stable to hydrolysis in the low pH of the stomach. Given that the solubility of the prodrugs of the invention is pH independent, crossing from the intestine having a low pH to the blood stream having a pH of around 7 will not cause the prodrugs to become soluble and dose dump. In a preferred embodiment, the orally delivered prodrugs further comprise a delivery system capable of enhancing sustained release and providing protection from enzymatic and chemical cleavage in the stomach and upper intestines. Additionally, such prodrug delivery system may comprise lipid-like features that facilitate uptake via lymph fluid, mitigating exposure to the liver on the way to the systemic circulation. This latter property can be advantageous for drugs that experience metabolism in the liver to metabolites that are undesirable due to inactivity and/or toxicity.

Other embodiments of the invention exploit the pH-independent aqueous solubility of the prodrugs of the invention. A key advantage of the prodrugs of the invention over their parent drugs is that the prodrug solubility remains essentially unchanged between pH 3 and 8, while the solubility of the tertiary amine parent drugs commonly increases by more than 100-fold over this pH range. The extent of solubilization accompanying pH reduction across this range depends on drug base solubility, pKa of the conjugate acid and counterions in the medium forming the ammonium salt. It is known in the art that biological tissues can become inflamed in response to injections, and that the pH of the inflamed tissue typically decreases from 7.1-7.4 down to pH 6.4 (See: A Dominant Role of Acid pH in Inflammatory Excitation and Sensitization of Nociceptors in Rat Skin, in vitro. Steen, K. H.; Steen, A. E.; Reeh, P. W. The Journal of Neuroscience, (1995), 15: pp. 3982-3989). Transiently pH in inflamed tissue can sometimes be as low as pH 4.7. Exercise alone can bring about a pH drop of about 0.5 units for up to 30 minutes (see: Continuous intramuscular pH measurement during the recovery from brief, maximal exercise in man. Allsop P; Cheetham M; Brooks S; Hall G M; Williams C. European journal of applied physiology and occupational physiology (1990), 59(6), pp. 465-70). It has also been demonstrated that release of drug from sustained release formulations can become rapid with reduced pH from subcutaneous space (see: Effect and interaction of pH and lidocaine on epinephrine absorption. Ueda, Wasa; Hirakawa, Masahisa; Mori, Koreaki, Anesthesiology (1988), 68(3), pp. 459-62), leading to a “burst” or “dumping” effect if the local pH drops at the injection site. It is hypothesized that this apparent failure of the formulations is caused by the high solubility of the drug at the lower pH. Therefore, even if the solubility of the prodrugs is similar to that of the corresponding parent tertiary amine at pH 7, the pH-independent solubility profiles of the prodrugs mean that solubility is controlled by the formulation without concern over dose-dumping in response to injection site irritation or, more generally, by pH fluctuations caused by patient activities, therapeutic interventions or illness.

Sustained release drug formulations often contain higher amounts of drugs than immediate release formulations. Functionality and safety of a sustained release formulation are based on a reliable and controlled rate of drug release from the formulation over an extended period of time after administration. The drug release profile of a formulation often depends on the chemical environment of the sustained release formulation, for example, on pH, ionic strength and presence of solvents such as ethanol.

The relatively high amount of drug that is present in a sustained release formulation can, in some instances, harm a patient if the formulation releases the drug at a rate that is faster than the intended controlled release rate. If the formulation releases the drug at a rate that is slower than the intended controlled release rate, the therapeutic efficacy of the drug can be reduced.

In most cases, partial or total failure of a sustained release formulation results in a rapid release of the drug into the bloodstream. This rapid release is generally faster than the intended sustained release of the drug from the formulation, and is sometimes referred to as “dose dumping.”

Dose dumping can create severe consequences for a patient, including permanent harm and even death. Examples of drugs that can be fatal if the therapeutically beneficial dose is exceeded, e.g., by dose dumping, include pain medications such as opioids, as well as other agents active in the central nervous system. In those situations where dose dumping may not be fatal, dose dumping may at least be responsible for the side effect of increased sedation of the patient as compared to administration of the parent drug alone (not in prodrug form).

The present invention solves the problem of dose dumping and its associated side effects, including but limited increased sedation, in a sustained release formulation by providing prodrugs that maintain their reduced solubility and sustained release action in a manner which is independent of the pH of the environment in which the prodrug is administered. The pH-independent solubility of the prodrugs of the invention is an important feature for drugs that are administered both orally and by injection. During oral administration, the prodrugs of the invention are exposed to a variety of pH conditions including very low pHs in the stomach (e.g. pH 1-2) and then increased pH when crossing the intestinal walls into the bloodstream. During injection it has been observed that the pH at the injection site may also be lowered (e.g. below pH 6.0). CRS 2009 Annual Meeting, Copenhagen Denmark, poster 242; Steen, K. H.; Steen, A. E.; Reeh, P. W. The Journal of Neuroscience, (1995), 15: pp. 3982-3989). The pH of an injection site may be lowered for a short amount of time (1-2 hours), but the perturbation may be sufficient to substantially dissolve a basic drug having pH-dependent solubility. In accordance with the invention, the reduced solubility of the prodrugs of the invention remains independent of any change in pH. In one preferred embodiment the reduced solubility of the prodrugs of the invention remains independent over a pH range of pH 4 to pH 8. More preferably the reduced solubility of the prodrugs of the invention remains independent over a pH range of pH 3 to pH 9. Most preferably, the reduced solubility of the prodrugs of the invention remains independent over a pH range of 1.0 to 10.

In addition, it is known that the stability of carboxyl ester linkages, such as those contemplated in the prodrugs of the invention, is dependent on pH with optimum stability occurring at around pH 4-5. If injection site pH fluctuates to a value lower than neutral pH of 7.4, then the stability of the prodrug is increased relative to neutral pH. This stability increase further reduces the risk of early release of active drug from the compound, and thus avoids dose dumping by way of accelerated chemical cleavage of the prodrug.

One aspect of the present invention provides a compound having the general Formula I:

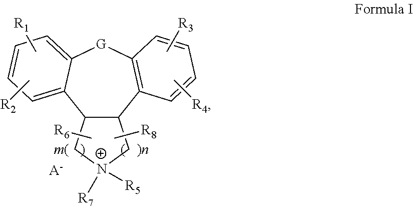

and geometric isomers, enantiomers, diastereomers, racemates, pharmaceutically acceptable salts and solvates thereof;
wherein each m and n is independently selected from 0, 1, 2 or 3;
each R₁, R₂, R₃, R₄, R₆, R₇, and R₈ is independently selected from absent, hydrogen, hydroxy, halogen, —OR₁₀, —SR₁₀, —N(R₁₀)(R₁₁)—, optionally substituted aliphatic, optionally substituted aryl or optionally substituted heterocyclyl;
wherein each R₁₀ and R₁₁ is independently hydrogen, halogen, aliphatic, substituted aliphatic, aryl or substituted aryl;
   alternatively, two of R₁, R₂, R₃ and R₄ together form an optionally substituted ring;
   R₅ is selected from —C(R₁₀)(R₁₁)—OR₁₂, —C(R₁₀)(R₁₁)—OC(O)OR₂₁, —C(R₁₀)(R₁₁)—OC(O)R₂₁, —C(R₁₀)(R₁₁)—OC(O)NR₁₂R₂₁, —C(R₁₀)(R₁₁)—OPO₃²⁻MY, —C(R₁₀)(R₁₁)—OP(O)(O⁻M)(OR₂₁), —C(R₁₀)(R₁₁)—OP(O)(OR₂₁)(OR₂₂);
wherein, each R₁₂ is independently hydrogen, halogen, aliphatic, substituted aliphatic, aryl or substituted aryl;
each R₂₁ and R₂₂ is independently hydrogen, aliphatic, substituted aliphatic, aryl or substituted aryl;
   G is selected from —O—, —S—, —NR₁₀, or —C(R₁₀)(R₁₁)—;
   Y and M are the same or different and each is a monovalent cation;

or M and Y together is a divalent cation; and

A- is a pharmaceutically acceptable counterion.

Optionally, the prodrug compound of formulas (I) or (II) further comprises a biocompatible delivery system for delivering the prodrug wherein the system is capable of minimizing accelerated hydrolytic cleavage of the prodrug by minimizing exposure of the prodrug to water. Preferred delivery systems include biocompatible polymeric matrix delivery systems capable of minimizing diffusion of water into the matrix and thereby minimizing exposure of the prodrug to bulk water during delivery. Substituents indicated as attached through variable points of attachments can be attached to any available position on the ring structure.

In another embodiment, compounds of the present invention are represented by Formula II as illustrated below, or its geometric isomers, enantiomers, diastereomers, racemates, pharmaceutically acceptable salts and solvates thereof:

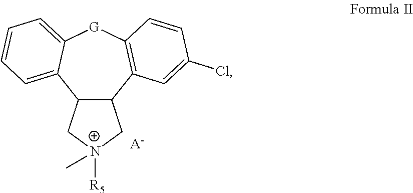

wherein R₅ and A- are as defined above.

In some embodiments, the R₅ is selected from:

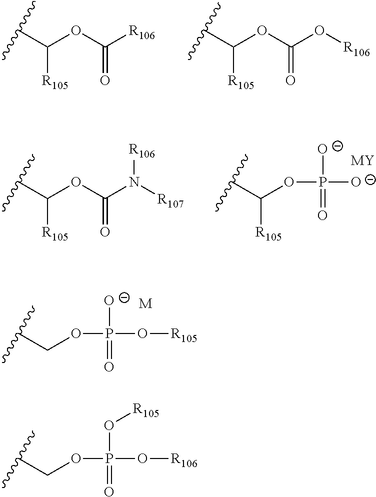

wherein R₁₀₅, R₁₀₆ and R₁₀₇ are independently selected from hydrogen, halogen, optionally substituted C₁-C₂₄ alkyl, optionally substituted C₂-C₂₄ alkenyl, optionally substituted C₂-C₂₄ alkynyl, optionally substituted C₃-C₂₄ cycloalkyl, optionally substituted C₁-C₂₄ alkoxy, optionally substituted C₁-C₂₄ alkylamino and optionally substituted C₁-C₂₄ aryl.

In some embodiments, R₅ is selected from:

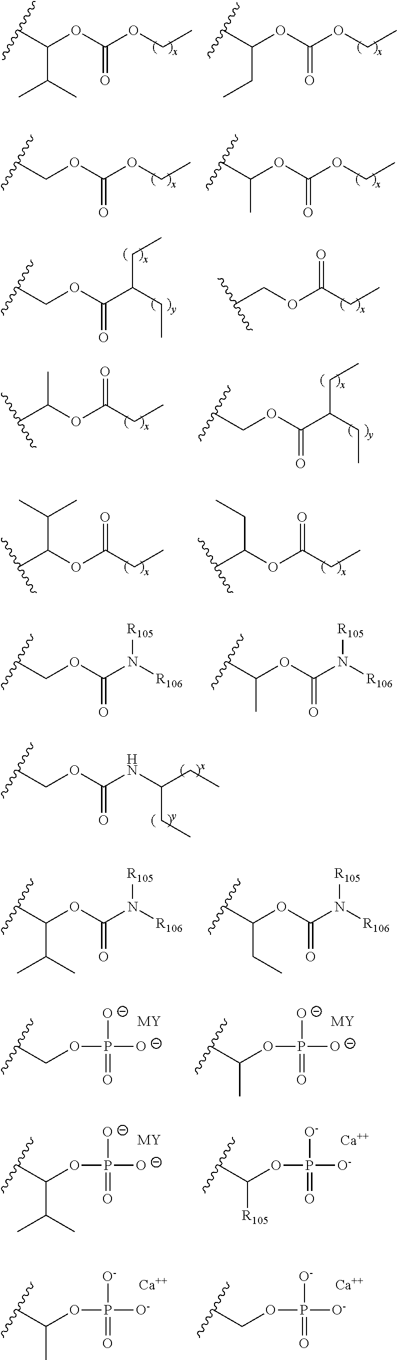

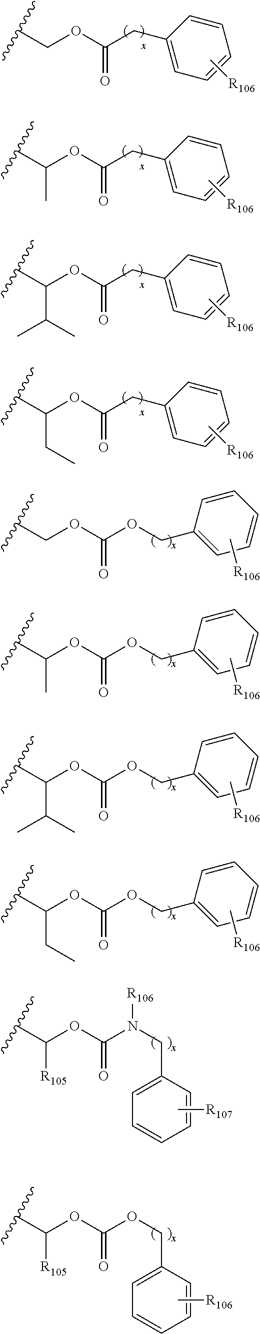

Wherein each x and y is independently an integer between 0 and 30; and M, Y, R₁₀₅, R₁₀₆, and R₁₀₇ are as defined above.

In certain embodiments, R₅ selected from:

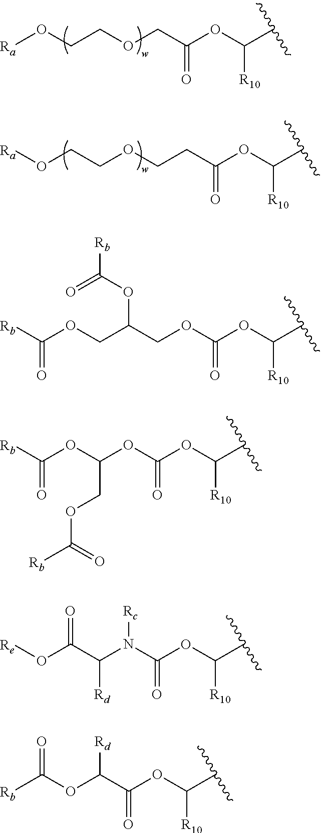

wherein w is 1 to about 1000, preferably 1 to about 100; Rₐ, R_{b} and Rₑ are each independently C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, aryl or substituted aryl; R_{c} is H or substituted or unsubstituted C₁-C₆-alkyl; R_{d} is H, substituted or unsubstituted C₁-C₆-alkyl, substituted or unsubstituted aryl-C₁-C₆-alkyl or substituted or unsubstituted heteroaryl-C₁-C₆-alkyl; and R₁₀ is as defined above and is preferably hydrogen. Preferably Rₐ, R_{b} and Rₑ are each C₁-C₂₄-alkyl.

Preferably R_{d} is the side chain of one of the twenty naturally occurring amino acids, more preferably a neutral or hydrophobic side chain, such as hydrogen, methyl, isopropyl, isobutyl, benzyl, indolylmethyl, and sec-butyl. R_{c} and R_{d} can also, together with the carbon and nitrogen atoms to which they are attached, form a heterocycloalkyl group, preferably a pyrrolidine group.

In some embodiments, x is an integer between 5 and 20. In a preferred embodiment, compounds or the geometric isomers, enantiomers, diastereomers, racemates, pharmaceutically acceptable salts or solvates thereof are selected from:

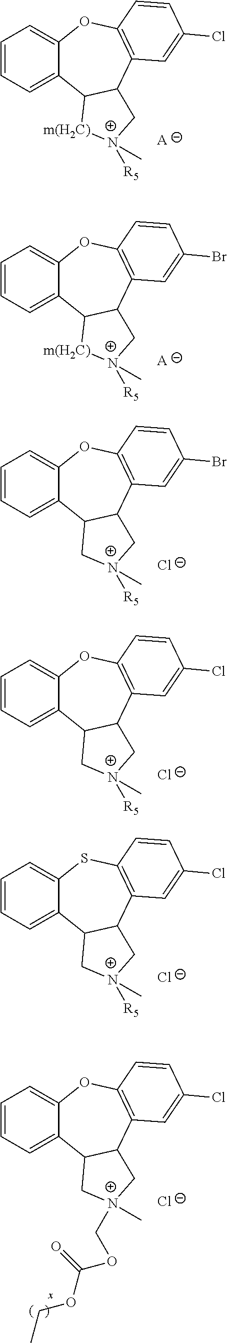

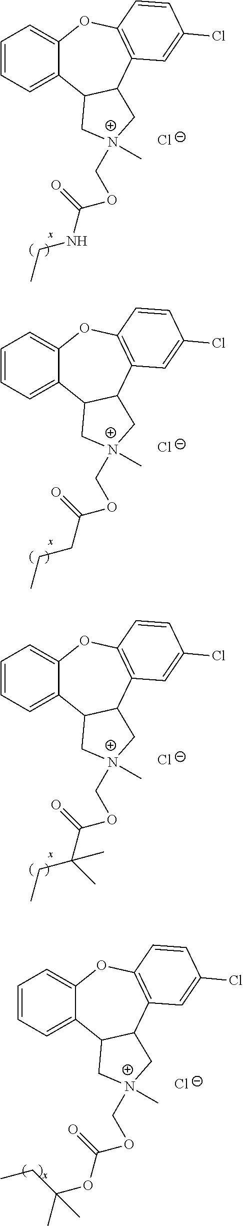

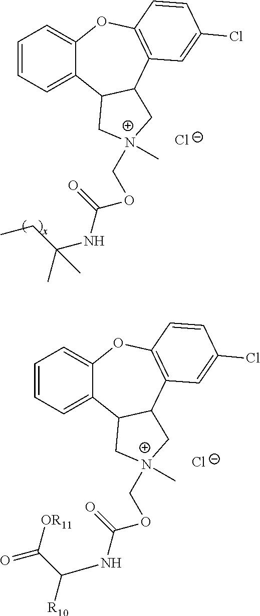

wherein x, m, R₅, R₁₀ and A⁻ are as defined above.

In one embodiment, variable R₅ in Formula I or Formula II and the geometric isomers, enantiomers, diastereomers, racemates, pharmaceutically acceptable salts or solvates thereof are selected from the group set forth in the Table 1 below, where the variables Y and M are the same or different and each is a monovalent cation, or M and Y together are a divalent cation. Compounds of formula I and II can form intramolecular or intermolecular salt bridges instead of associating with other counterions. For example, compounds of Formula I and II in which R₅ is —C(R₈)(R₉)—OPO₃MY or —CH(R₈)(R₉)—OP(O)₂(OR₁₁)M, it is possible for the phosphate moiety to serve as X— and for the quaternary ammonium group to serve as M.

TABLE 1

| | |
|---|---|
| 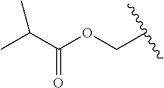 | 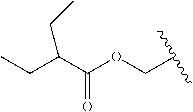 |

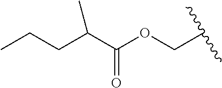

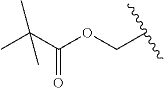

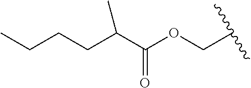

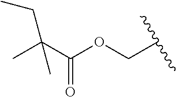

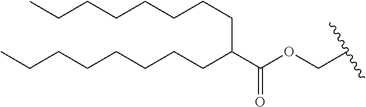

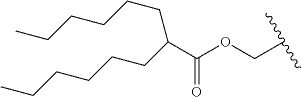

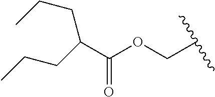

| | |
|---|---|
| 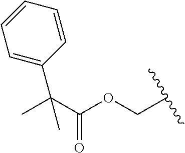 | 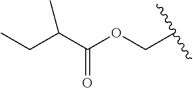 |
| 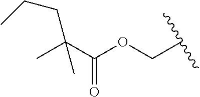 | 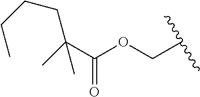 |

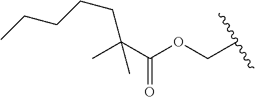

| | |
|---|---|
| 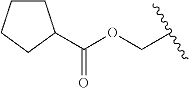 | 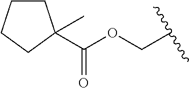 |
| 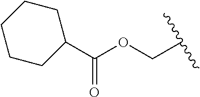 | 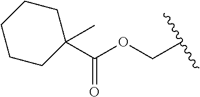 |
| 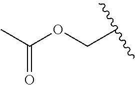 | 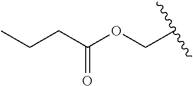 |

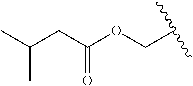

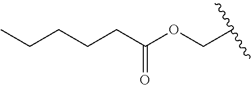

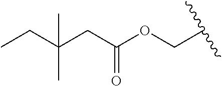

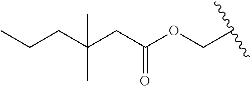

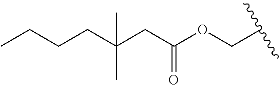

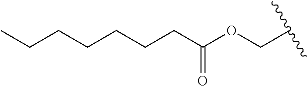

| | |
|---|---|
| 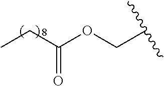 | 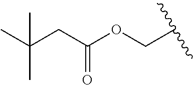 |
| 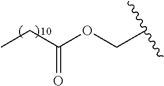 | 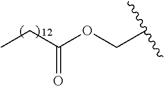 |
| 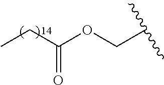 | 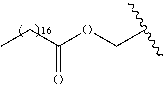 |
| 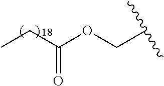 | 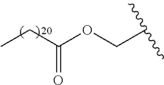 |
| 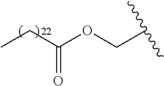 | 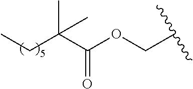 |
| 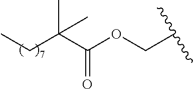 | 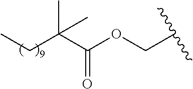 |
| 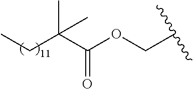 | 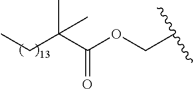 |
| 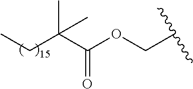 | 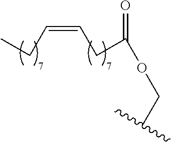 |
| 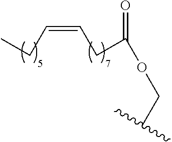 | 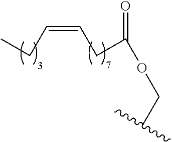 |

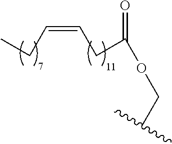

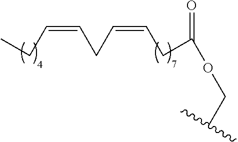

| | |
|---|---|
| 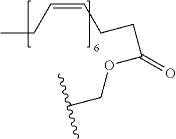 | 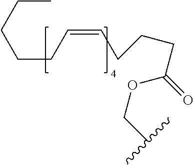 |

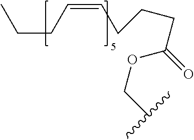

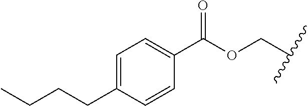

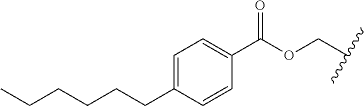

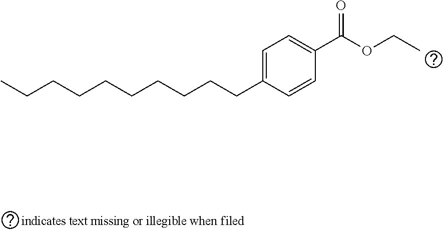

| | |
|---|---|
| 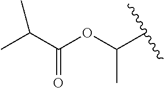 | 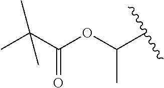 |
| 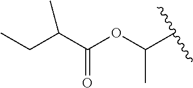 | 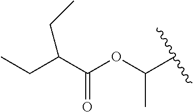 |

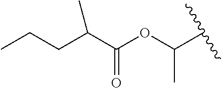

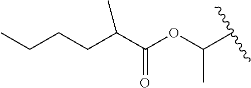

| | |
|---|---|
| 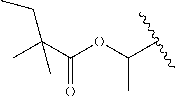 | 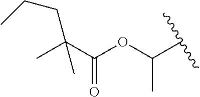 |

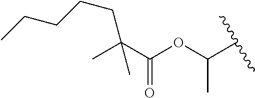

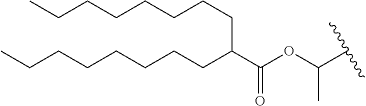

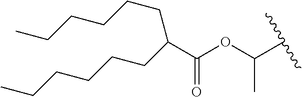

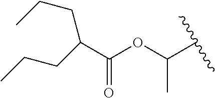

| | |
|---|---|
| 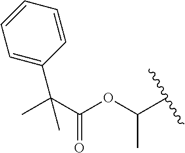 | 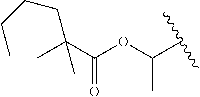 |
| 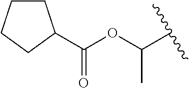 | 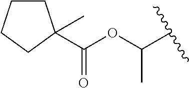 |
| 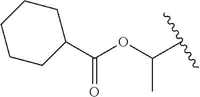 | 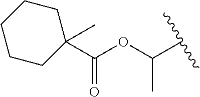 |
| 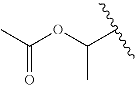 | 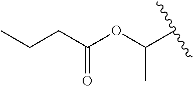 |
| 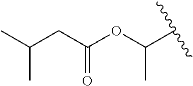 | 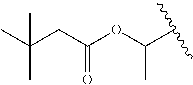 |

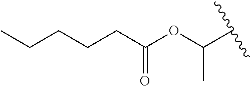

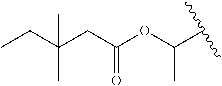

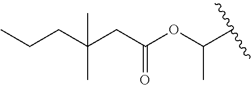

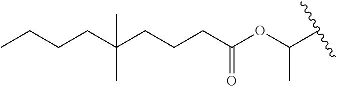

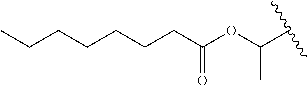

| | |
|---|---|
| 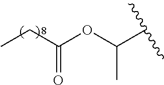 | 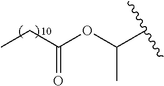 |
| 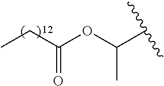 | 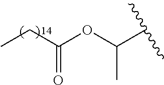 |
| 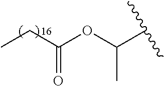 | 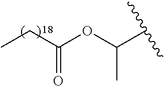 |
| 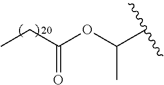 | 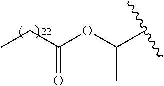 |
| 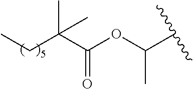 | 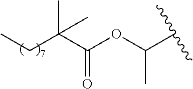 |
| 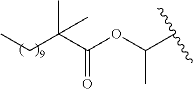 | 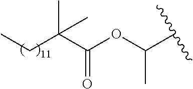 |
| 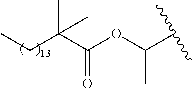 | 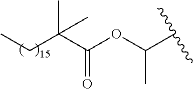 |
| 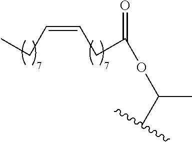 | 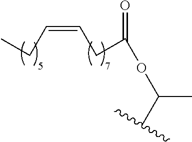 |
| 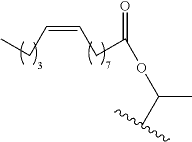 | 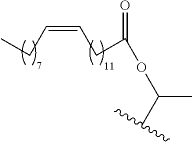 |

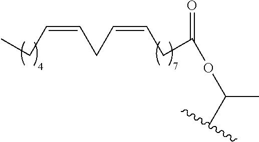

| | |
|---|---|
| 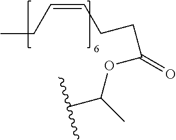 | 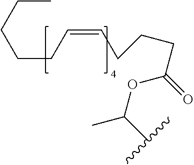 |

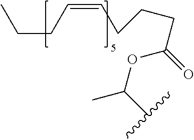

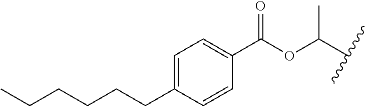

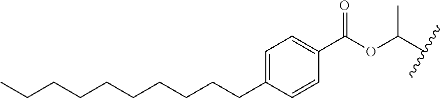

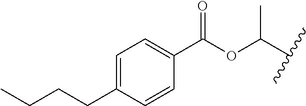

| | |
|---|---|
| 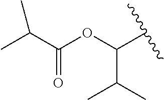 | 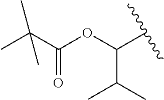 |
| 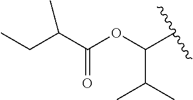 | 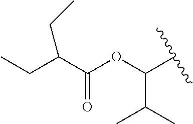 |

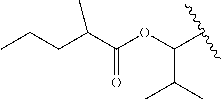

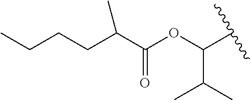

| | |
|---|---|
| 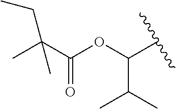 | 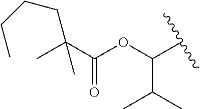 |

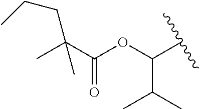

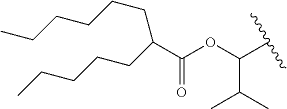

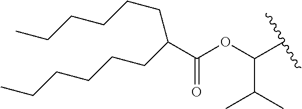

| | |
|---|---|
| 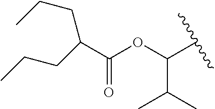 | 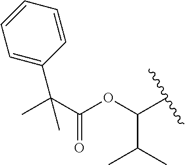 |

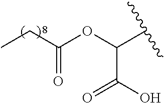

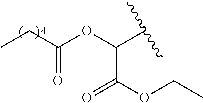

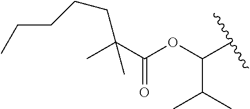

| | |
|---|---|
| 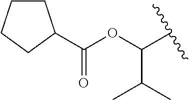 | 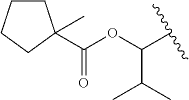 |
| 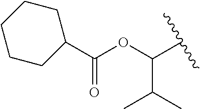 | 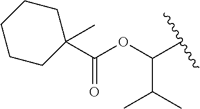 |
| 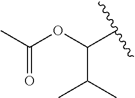 | 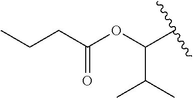 |
| 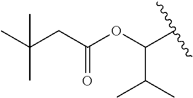 | 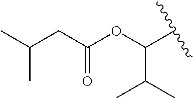 |

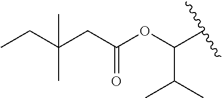

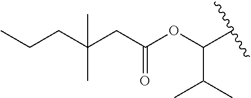

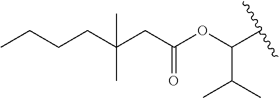

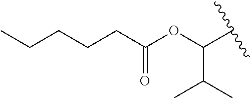

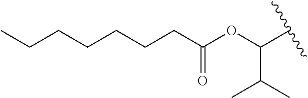

| | |
|---|---|
| 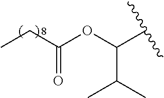 | 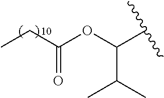 |
| 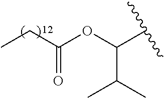 | 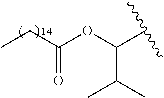 |
| 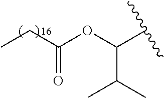 | 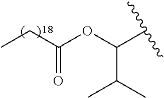 |
| 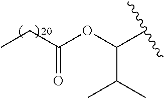 | 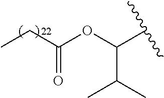 |
| 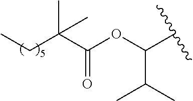 | 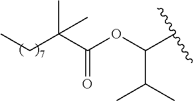 |
| 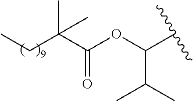 | 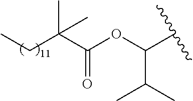 |
| 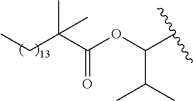 | 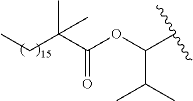 |

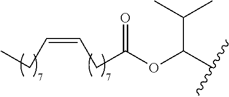

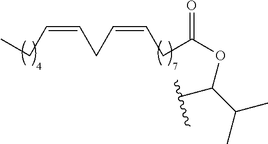

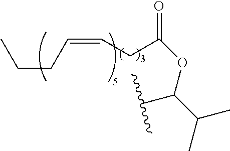

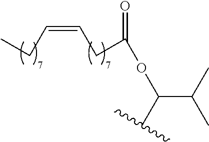

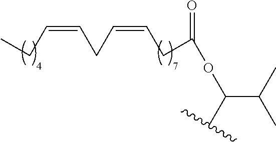

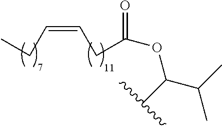

| | |
|---|---|
| 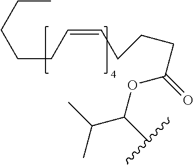 | 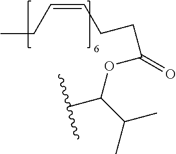 |

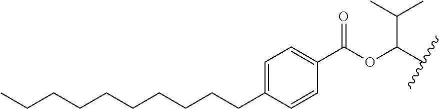

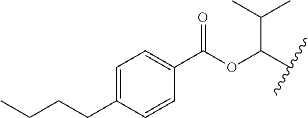

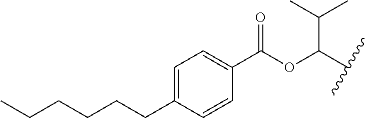

| | |
|---|---|
| 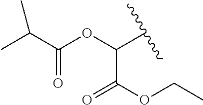 | 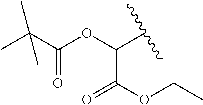 |

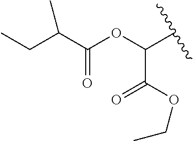

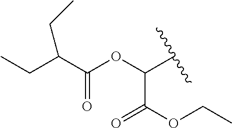

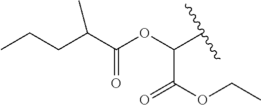

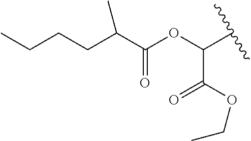

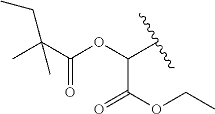

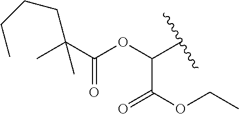

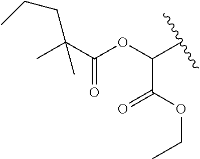

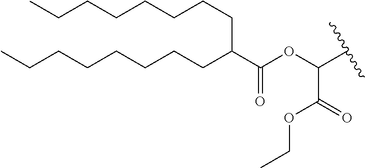

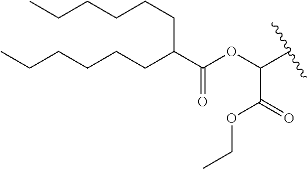

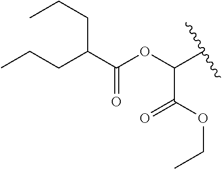

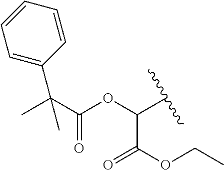

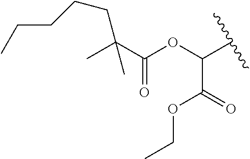

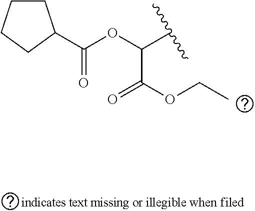

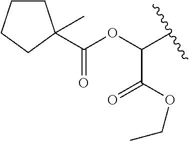

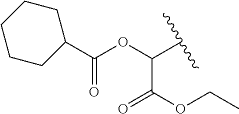

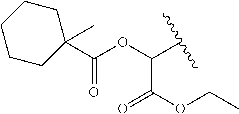

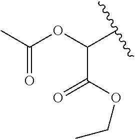

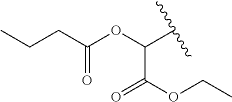

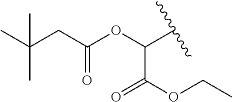

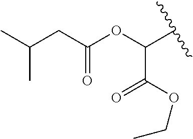

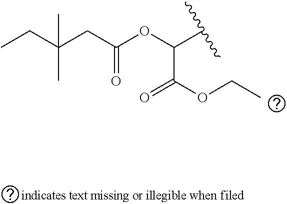

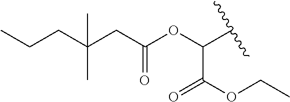

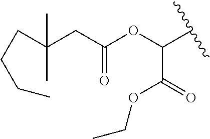

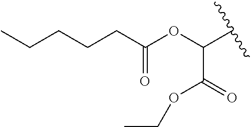

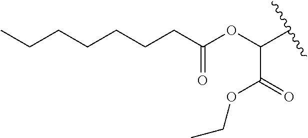

| | |
|---|---|
| 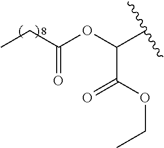 | 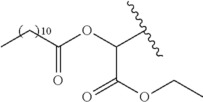 |
| 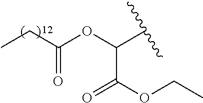 | 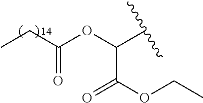 |
| 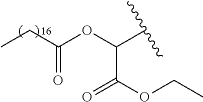 | 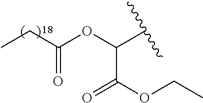 |
| 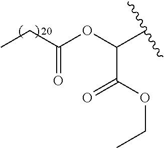 | 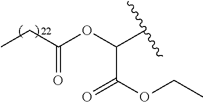 |

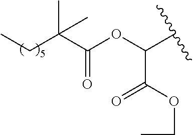

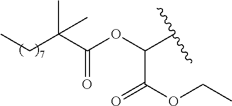

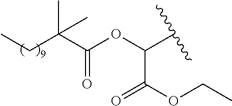

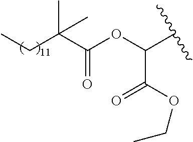

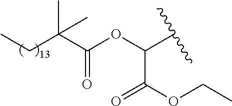

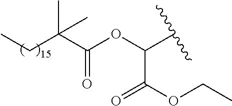

| | |
|---|---|
| 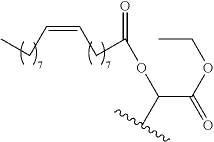 | 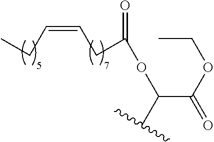 |

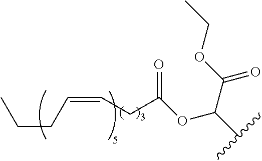

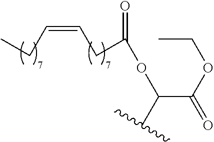

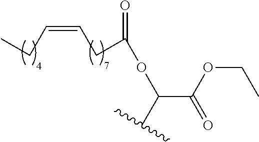

| | |
|---|---|
| 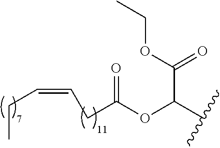 | 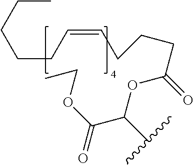 |

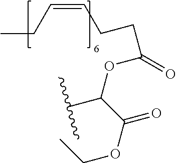

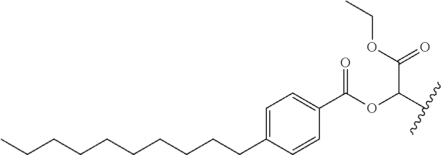

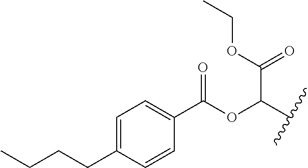

| | |
|---|---|
| 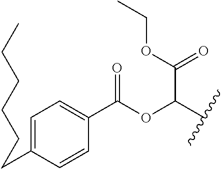 | 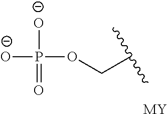 |
| 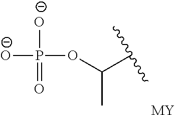 | 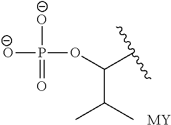 |
| 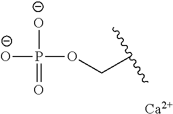 | 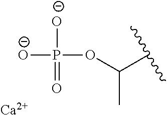 |
| 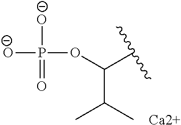 | 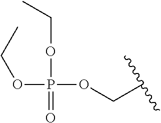 |
| 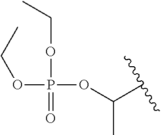 | 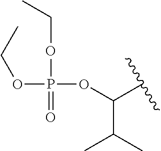 |

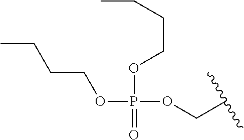

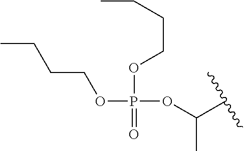

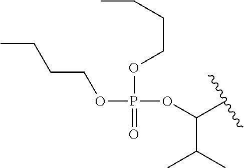

| | |
|---|---|
| 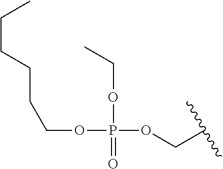 | 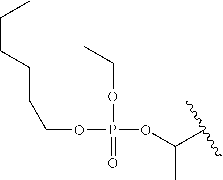 |

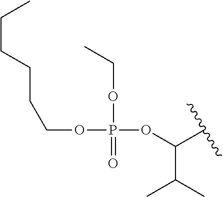

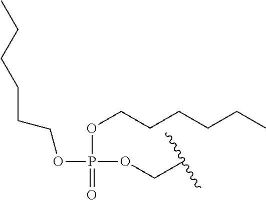

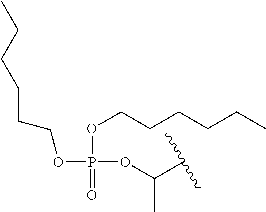

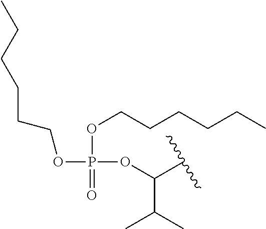

| | |
|---|---|
| 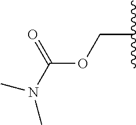 | 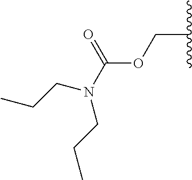 |
| 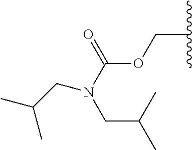 | 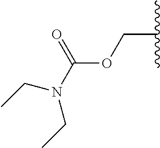 |
| 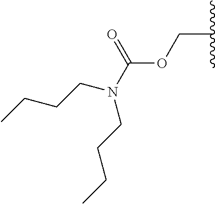 | 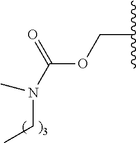 |
| 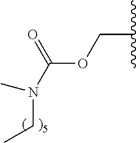 | 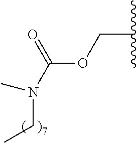 |

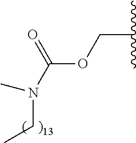

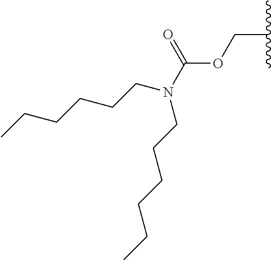

| | |
|---|---|
| 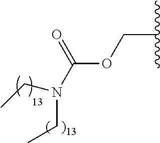 | 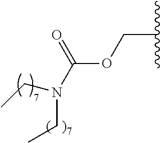 |
| 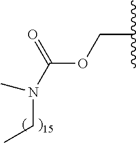 | 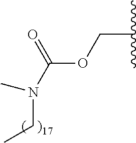 |
| 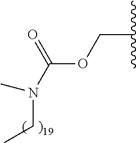 | 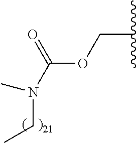 |
| 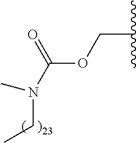 | 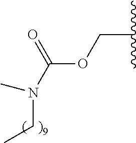 |

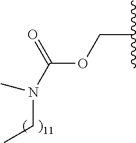

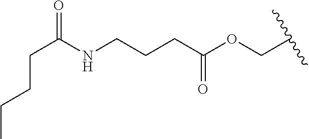

| | |
|---|---|
| 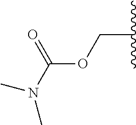 | 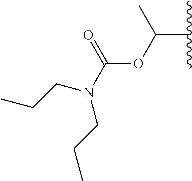 |

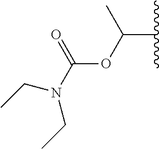

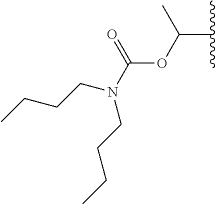

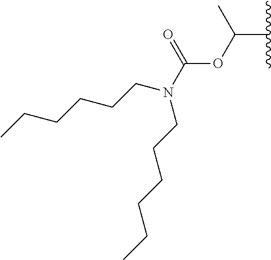

| | |
|---|---|
| 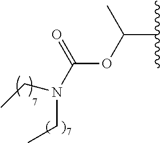 | 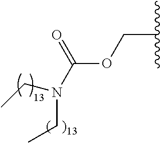 |
| 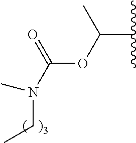 | 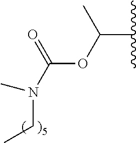 |
| 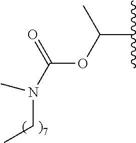 | 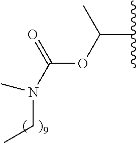 |
| 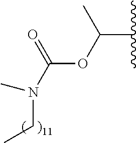 | 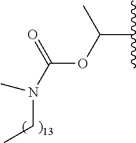 |
| 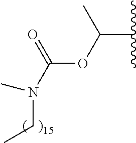 | 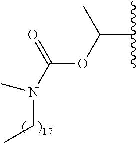 |
| 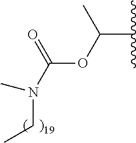 | 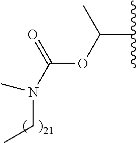 |
| 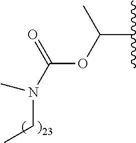 | 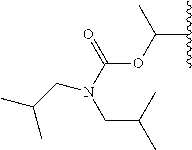 |
| 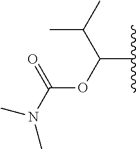 | 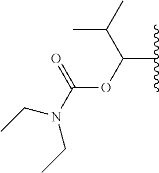 |
| 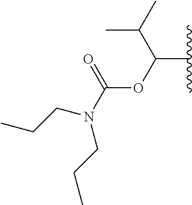 | 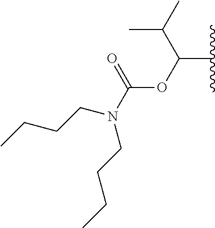 |

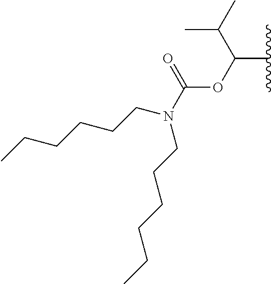

| | |
|---|---|
| 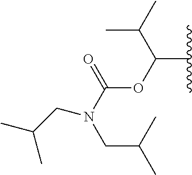 | 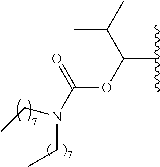 |
| 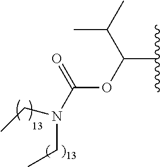 | 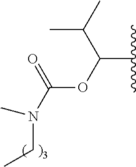 |
| 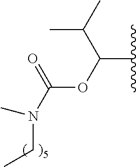 | 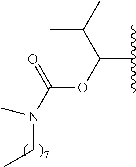 |
| 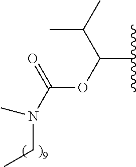 | 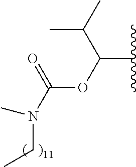 |
| 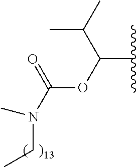 | 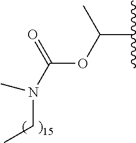 |
| 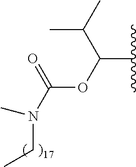 | 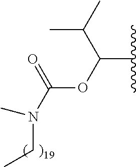 |
| 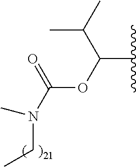 | 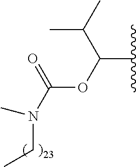 |

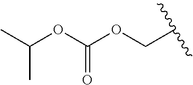

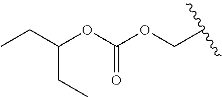

| | |
|---|---|
| 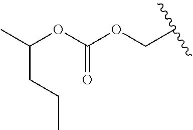 | 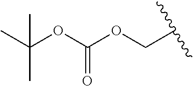 |

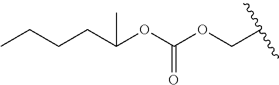

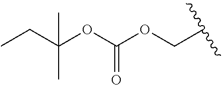

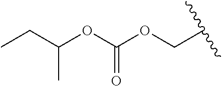

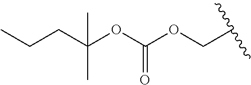

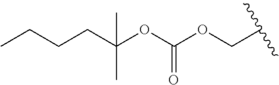

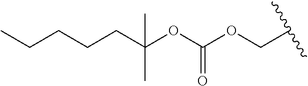

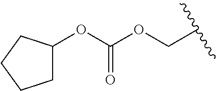

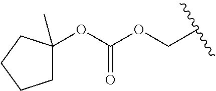

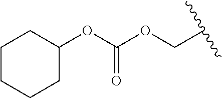

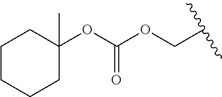

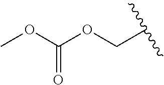

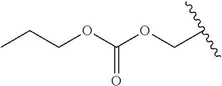

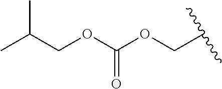

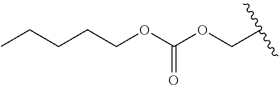

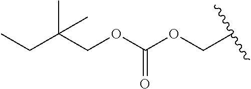

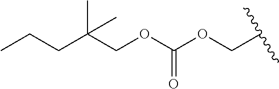

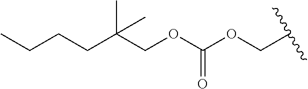

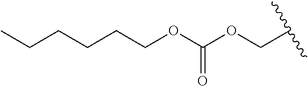

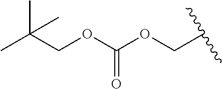

| | |
|---|---|
| 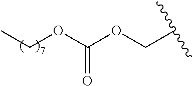 | 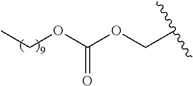 |
| 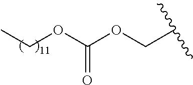 | 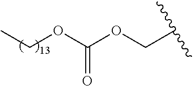 |
| 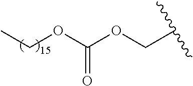 | 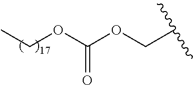 |
| 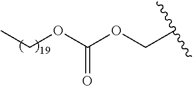 | 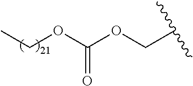 |

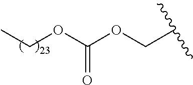

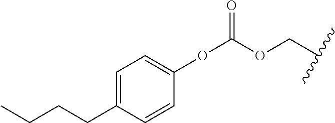

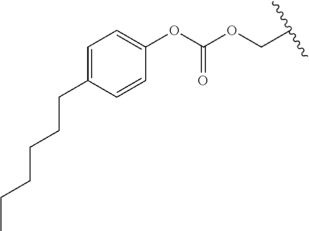

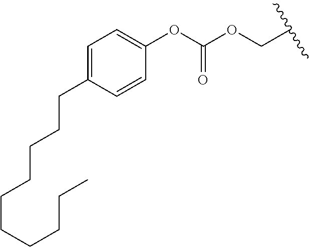

| | |
|---|---|
| 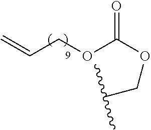 | 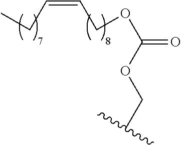 |
| 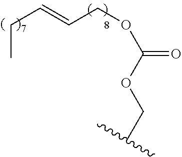 | 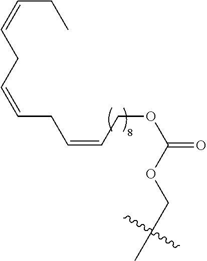 |
| 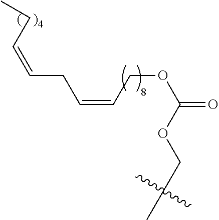 | 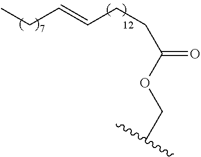 |
| 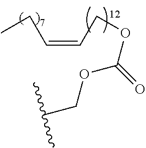 | 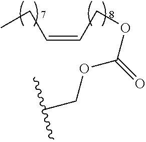 |
| 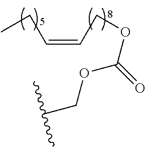 | 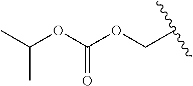 |

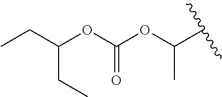

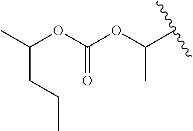

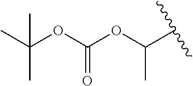

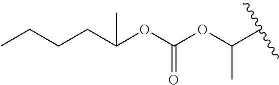

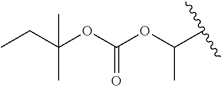

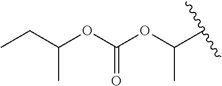

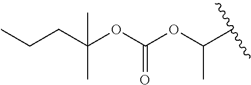

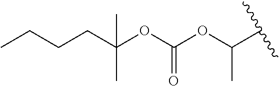

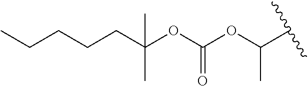

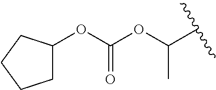

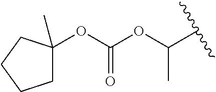

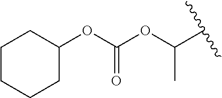

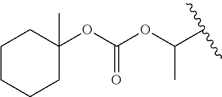

| | |
|---|---|
| 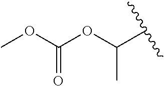 | 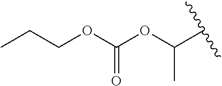 |

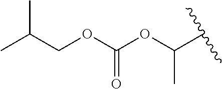

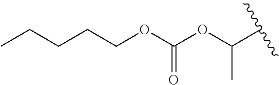

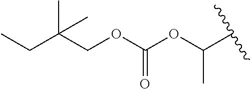

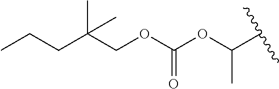

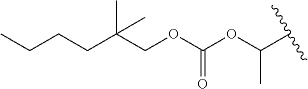

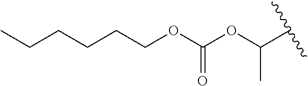

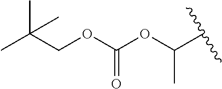

| | |
|---|---|
| 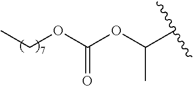 | 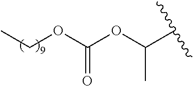 |
| 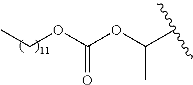 | 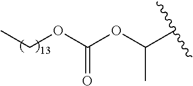 |
| 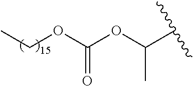 | 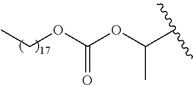 |
| 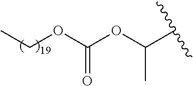 | 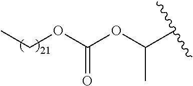 |

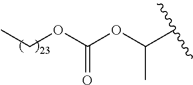

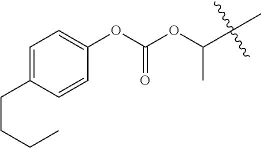

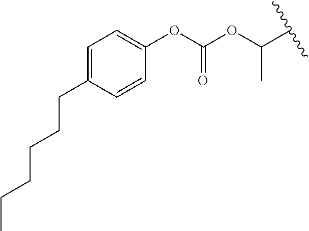

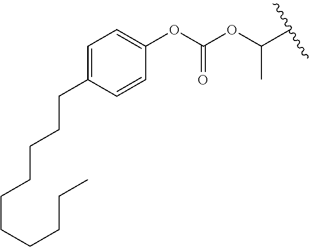

| | |
|---|---|
| 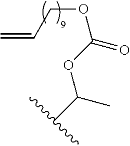 | 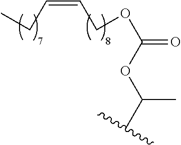 |
| 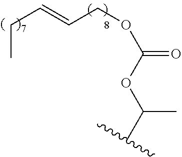 | 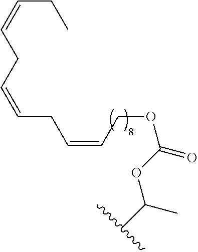 |
| 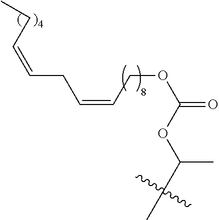 | 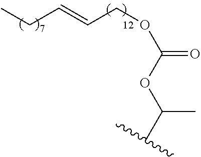 |
| 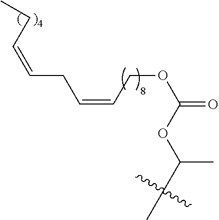 | 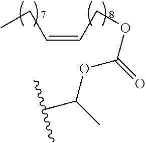 |
| 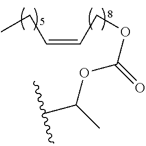 | 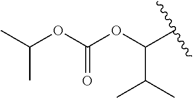 |

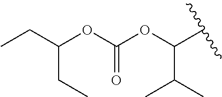

| | |
|---|---|
| 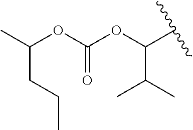 | 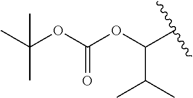 |

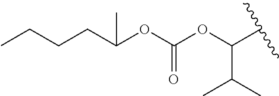

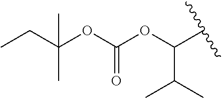

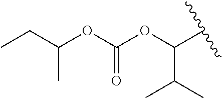

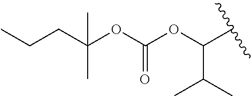

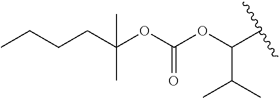

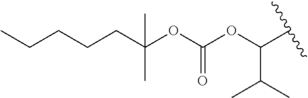

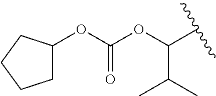

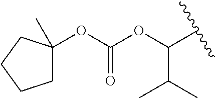

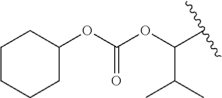

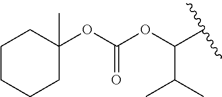

| | |
|---|---|
| 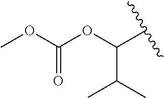 | 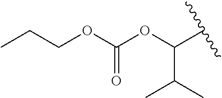 |

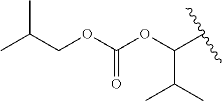

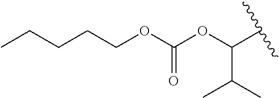

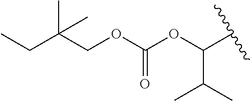

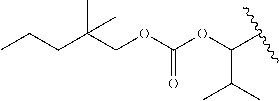

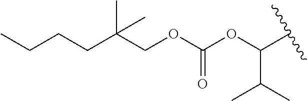

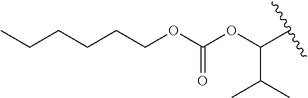

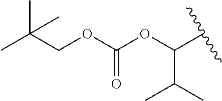

| | |
|---|---|
| 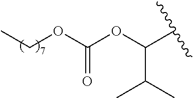 | 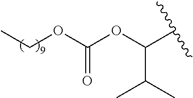 |
| 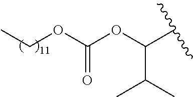 | 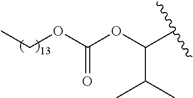 |
| 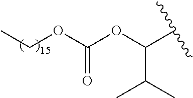 | 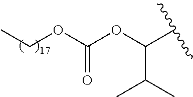 |
| 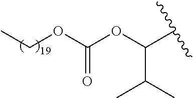 | 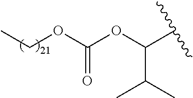 |

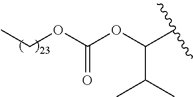

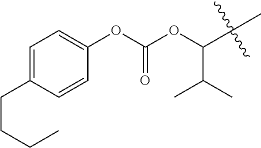

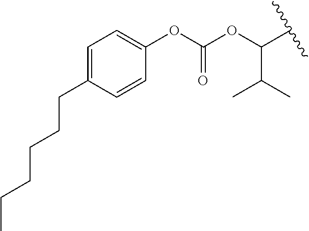

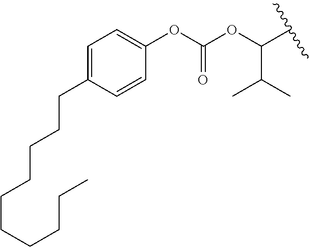

| | |
|---|---|
| 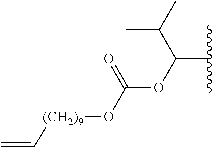 | 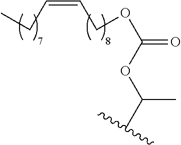 |
| 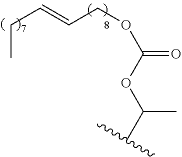 | 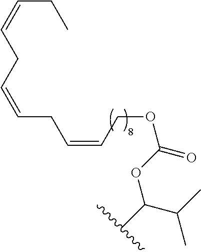 |

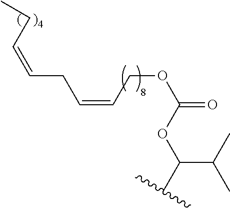

| | |
|---|---|
| 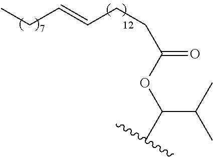 | 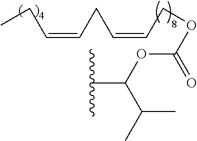 |
| 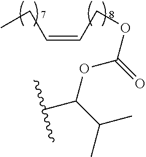 | 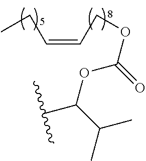 |

In one embodiment, R₅ in any of Formulae I and II is selected from the group set forth in Tables 2, 3, 4 or 5 below.

TABLE 2

| | |
|---|---|
| 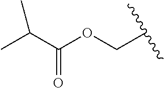 | 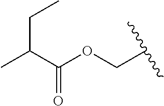 |

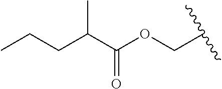

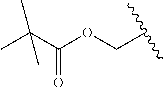

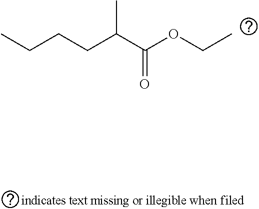

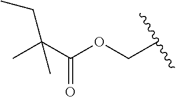

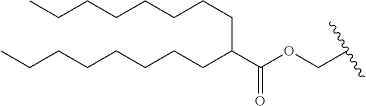

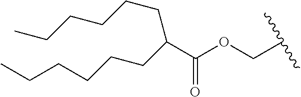

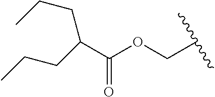

| | |
|---|---|
| 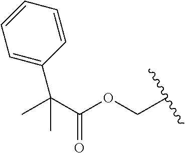 | 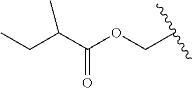 |
| 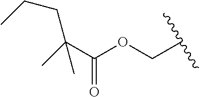 | 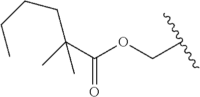 |

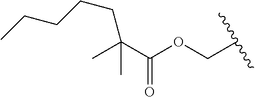

| | |
|---|---|
| 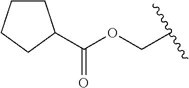 | 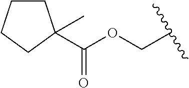 |
| 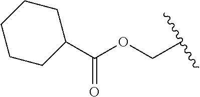 | 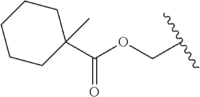 |
| 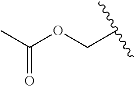 | 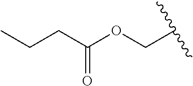 |

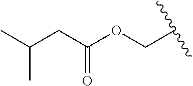

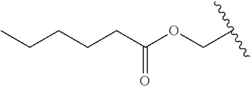

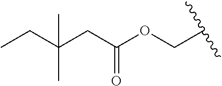

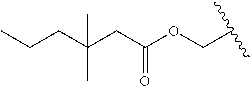

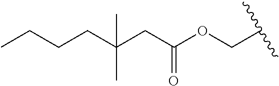

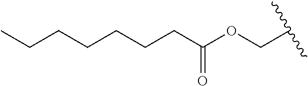

| | |
|---|---|
| 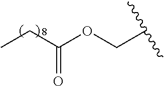 | 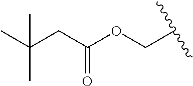 |
| 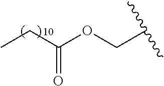 | 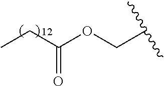 |
| 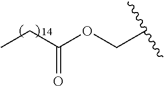 | 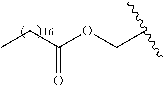 |
| 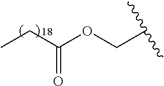 | 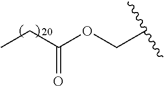 |
| 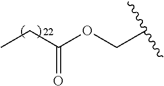 | 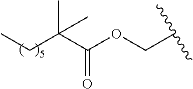 |
| 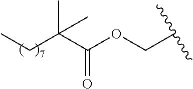 | 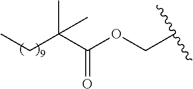 |
| 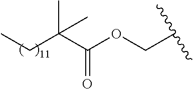 | 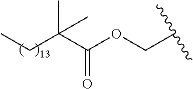 |
| 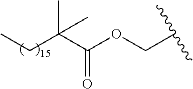 | 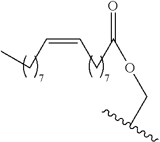 |
| 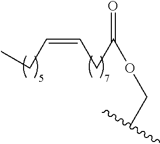 | 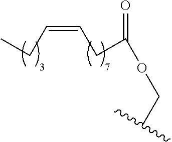 |

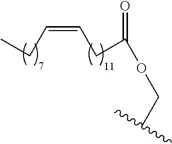

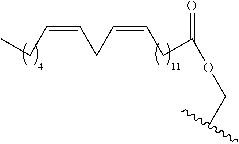

| | |
|---|---|
| 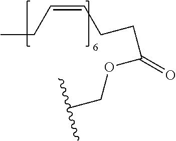 | 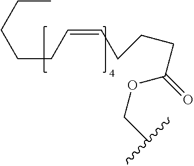 |

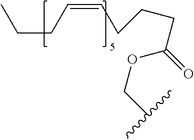

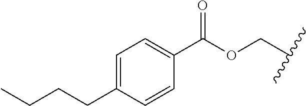

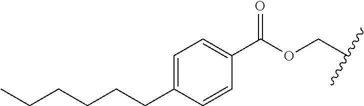

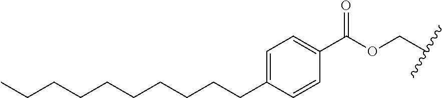

TABLE 3

| 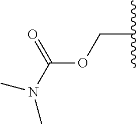 | 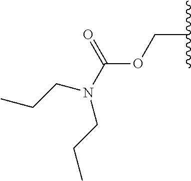 |
|---|---|
| 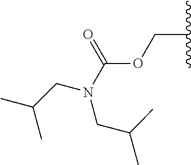 | 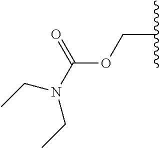 |
| 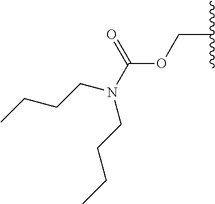 | |
| 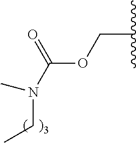 | 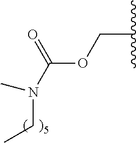 |
| 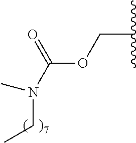 | 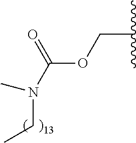 |
| 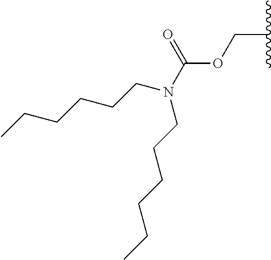 | |
| 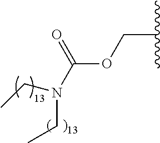 | 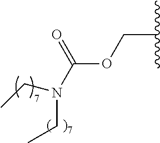 |
| 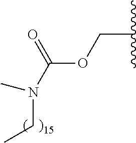 | 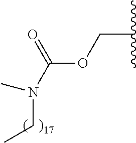 |
| 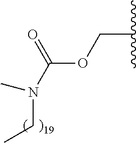 | 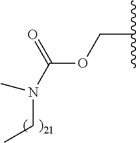 |
| 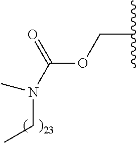 | 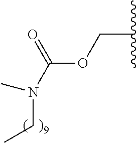 |

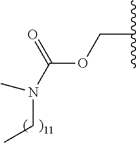

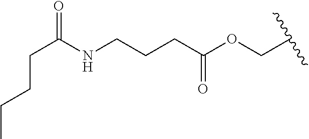

TABLE 4

| 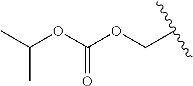 | |
|---|---|
| 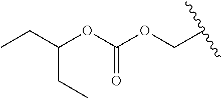 | |
| 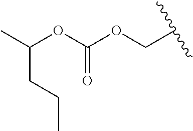 | |
| 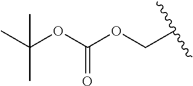 | |
| 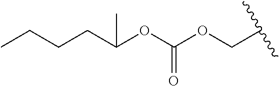 | |
| 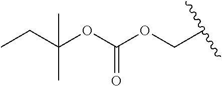 | |
| 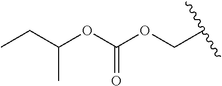 | |
| 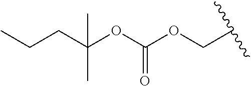 | |
| 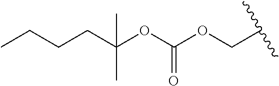 | |
| 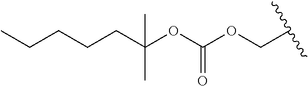 | |
| 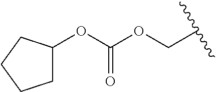 | |
| 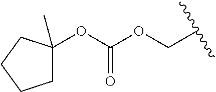 | |
| 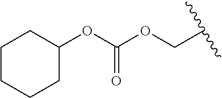 | |
| 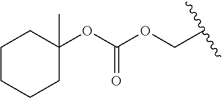 | |
| 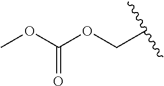 | |
| 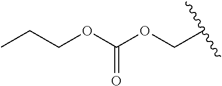 | |
| 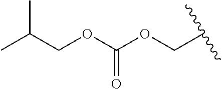 | |
| 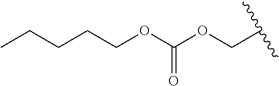 | |
| 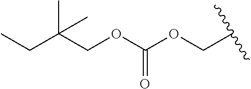 | |
| 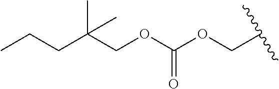 | |
| 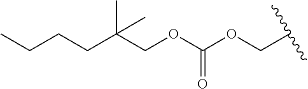 | |
| 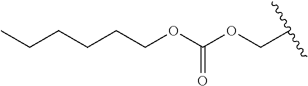 | |
| 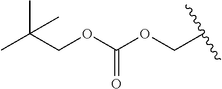 | |
| 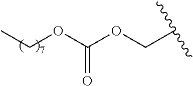 | |
| 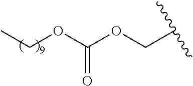 | |
| 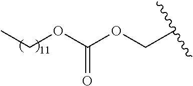 | |
| 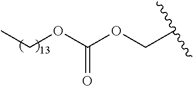 | |
| 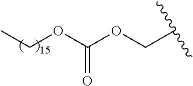 | |
| 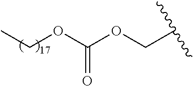 | |
| 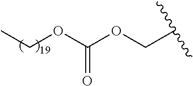 | |
| 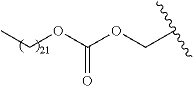 | |
| 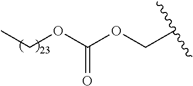 | |
| 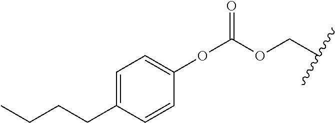 | |
| 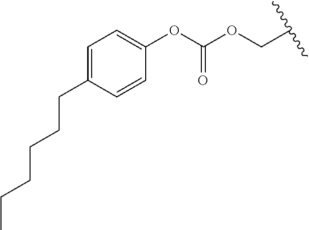 | |
| 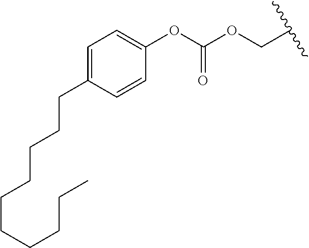 | |
| 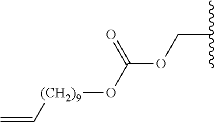 | |
| 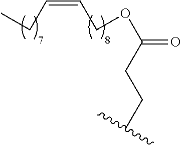 | |
| 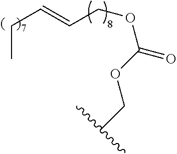 | |
| 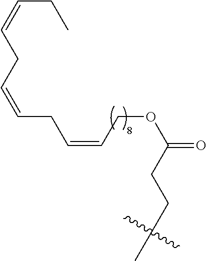 | |
| 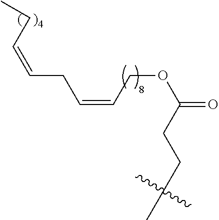 | |
| 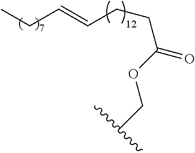 | |
| 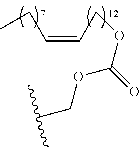 | 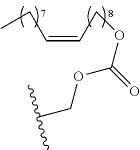 |

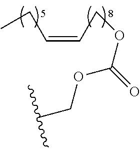

TABLE 5

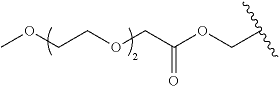

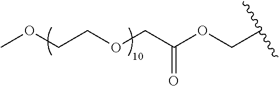

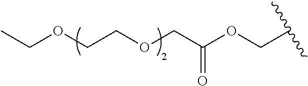

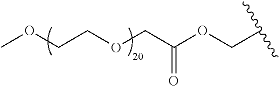

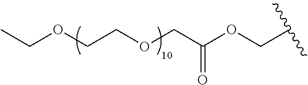

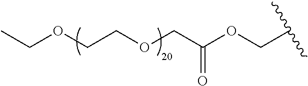

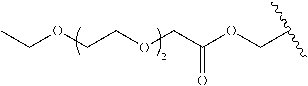

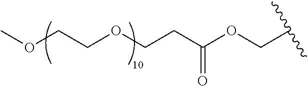

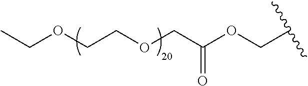

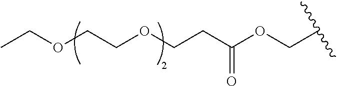

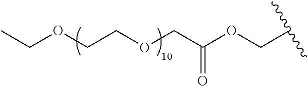

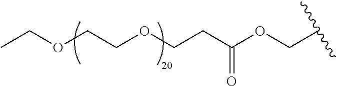

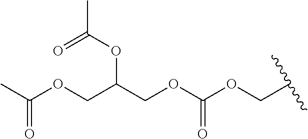

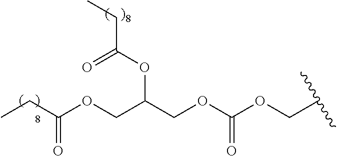

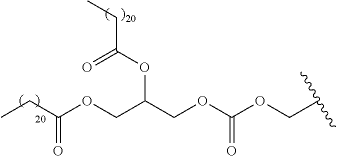

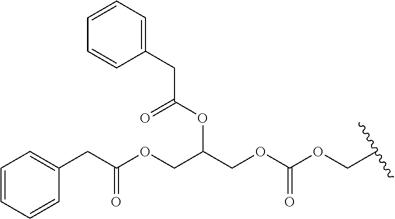

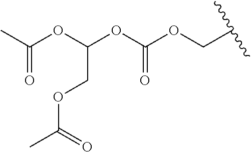

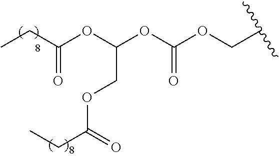

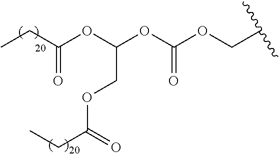

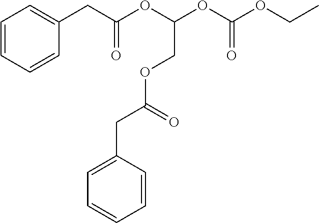

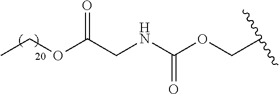

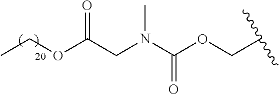

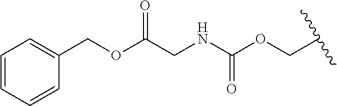

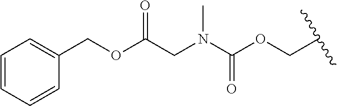

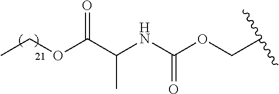

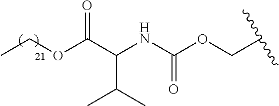

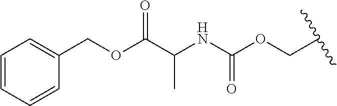

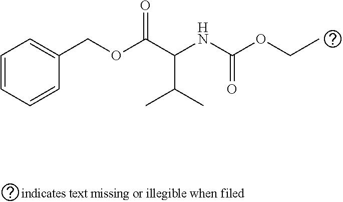

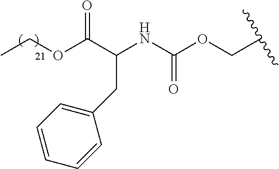

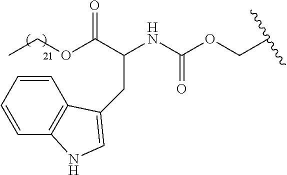

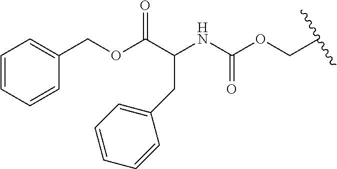

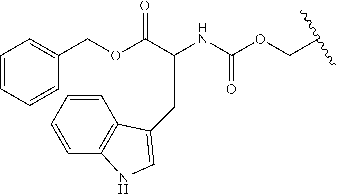

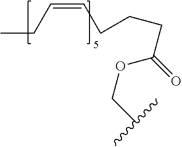

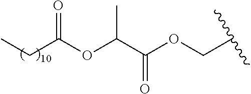

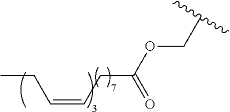

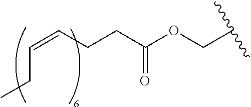

A preferred compound is a compound of having the formula:

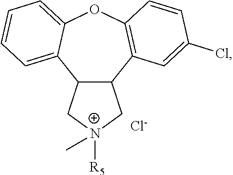

wherein R₅ is selected from Table 1.

Another preferred compound is a compound of having the formula:

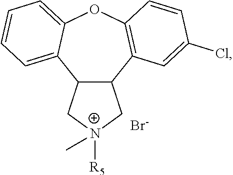

wherein R₅ is selected from Table 1.

In compounds of the invention, the quaternized nitrogen atom is a chiral center and both stereoisomers are converted in vivo to yield the parent drug. Such compounds can be formulated and used as a mixture of stereoisomers or as a composition having a single stereoisomer or a mixture with excess of one stereoisomer. In certain compounds the parent drug, such as asenapine, is chiral and can be used as a racemic mixture, containing two enantiomers with trans relative stereochemistry between the two chiral centers. For such a racemic mixture, quaternization of the nitrogen atom produces an additional chiral center and up to four stereoisomers. Such compounds can be formulated and used as a mixture of four stereoisomers. Alternatively, the diastereomers are separated to yield pairs of enantiomers, and a racemic mixture of one pair of enantiomers is formulated and used. In another embodiment, a single stereoisomer is formulated and used. Additionally it is possible to separate the two enantiomers of Asenapine. Quaternization of a single enantiomer of Asenapine will provide two diastereomer products that can be either formulated and used as a mixture or separated and formulated and used as a single stereoisomer. Unless otherwise stated, the structural formula of a compound herein is intend to represent all enantiomers, racemates and diastereomers of that compound.

Representative compounds according to the invention are those selected from the Table A below and, enantiomers, diastereomers, racemates, pharmaceutically acceptable salts, and prodrugs thereof. Some of the salts are represented as chloride, bromide, iodide, calcium or acetate salts; however the compounds can be prepared as salts of other pharmaceutically acceptable anions. Selection of a suitable anion can be made on a case-by-case basis to modulate the solubility and/or delivery properties of the material.

TABLE A

| No. | Structure |
|---|---|
| 1 | 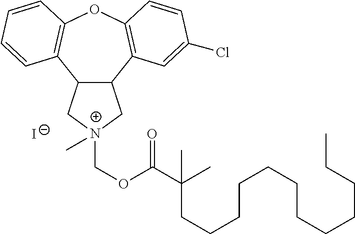 |
| 2 | 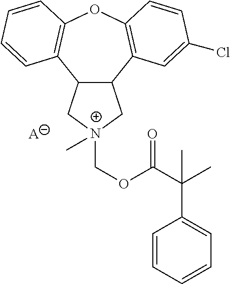 |
| 3 | 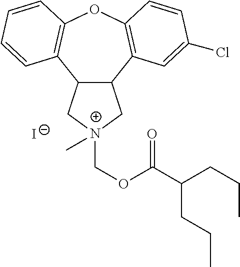 |
| 4 | 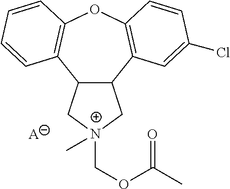 |
| 5 | 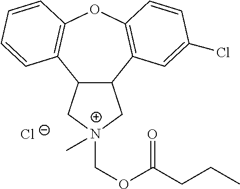 |
| 6 | 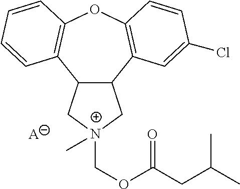 |
| 7 | 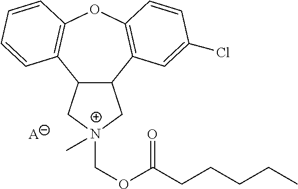 |
| 8 | 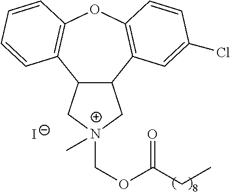 |
| 9 | 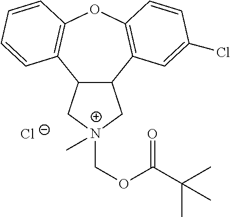 |
| 10 | 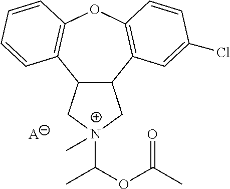 |
| 11 | 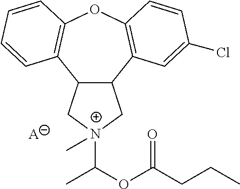 |
| 12 | 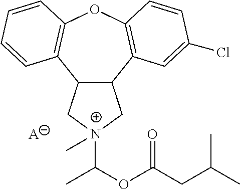 |
| 13 | 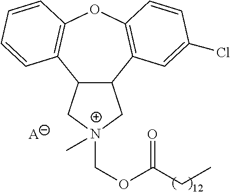 |
| 14 | 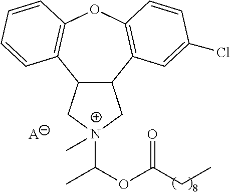 |
| 15 | 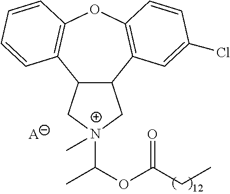 |
| 16 | 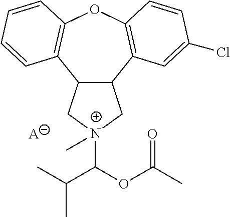 |
| 17 | 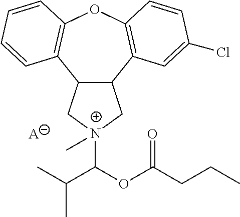 |
| 18 | 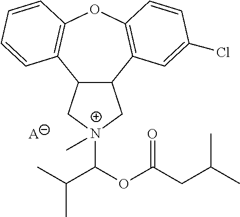 |
| 19 | 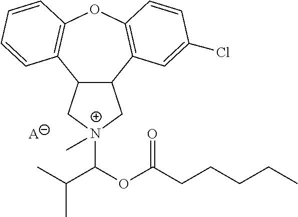 |
| 20 | 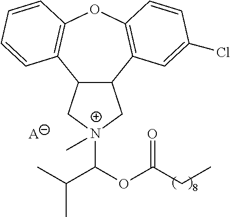 |
| 21 | 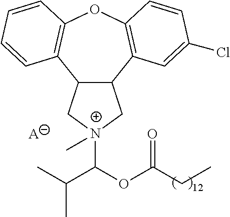 |
| 22 | 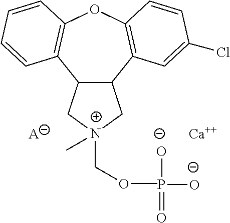 |
| 23 | 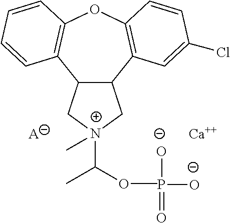 |
| 24 | 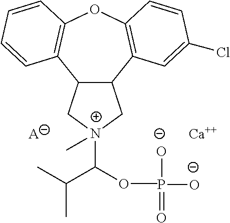 |
| 25 | 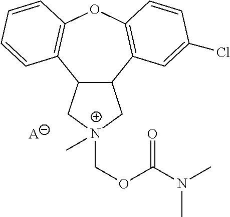 |
| 26 | 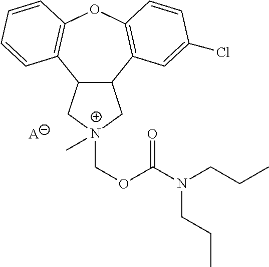 |
| 27 | 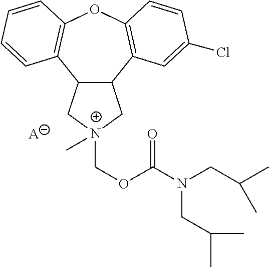 |
| 28 | 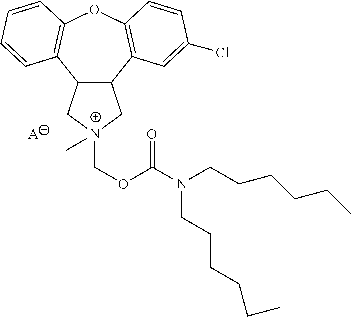 |
| 29 | 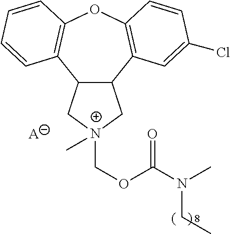 |
| 30 | 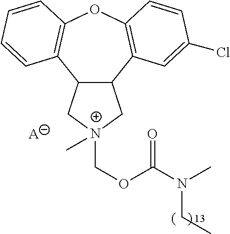 |
| 31 | 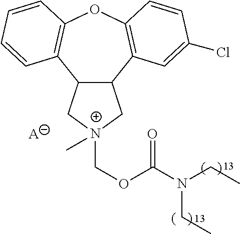 |
| 32 | 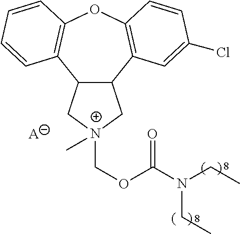 |
| 33 | 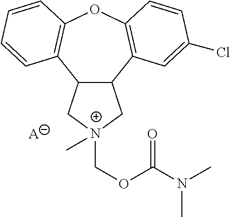 |
| 34 | 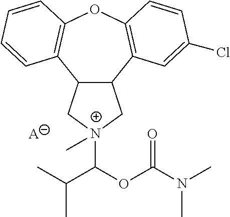 |
| 35 | 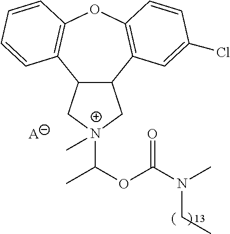 |
| 36 | 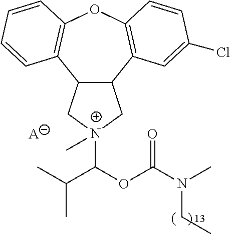 |
| 37 | 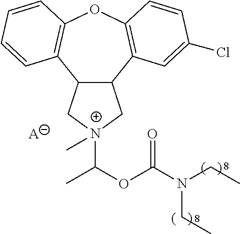 |
| 38 | 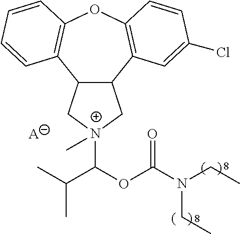 |
| 39 | 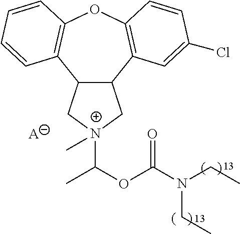 |
| 40 | 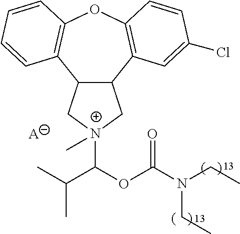 |
| 41 | 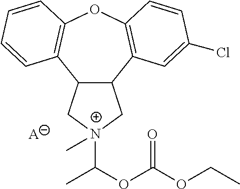 |
| 42 | 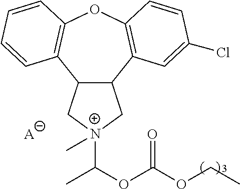 |
| 43 | 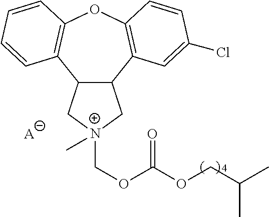 |
| 44 | 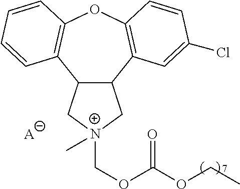 |
| 45 | 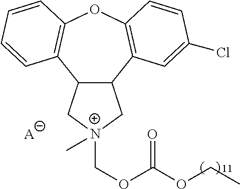 |
| 46 | 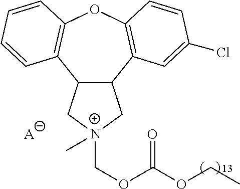 |
| 47 | 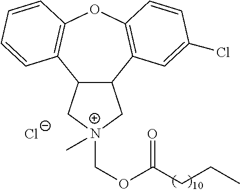 |
| 48 | 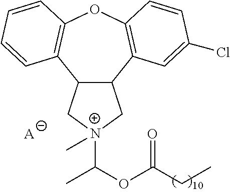 |
| 49 | 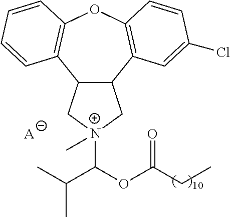 |
| 50 | 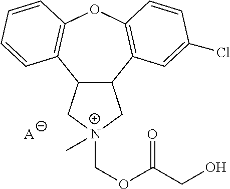 |
| 51 | 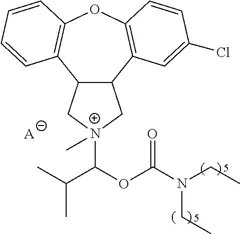 |
| 52 | 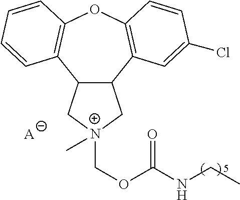 |
| 53 | 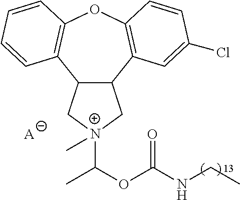 |
| 54 | 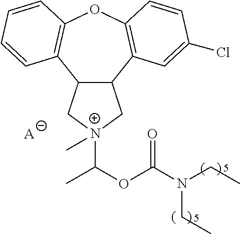 |
| 55 | 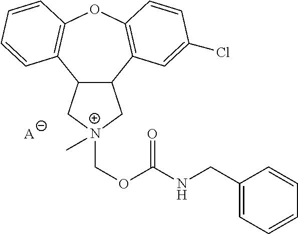 |
| 56 | 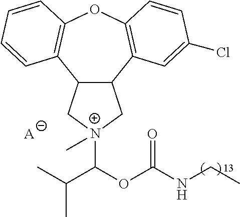 |
| 57 | 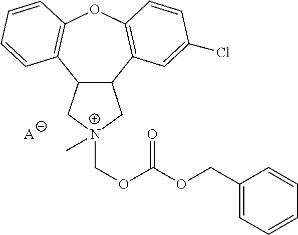 |
| 58 | 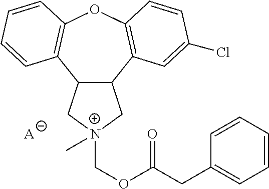 |
| 59 | 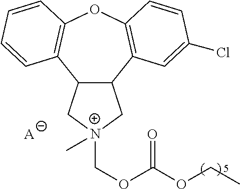 |
| 60 | 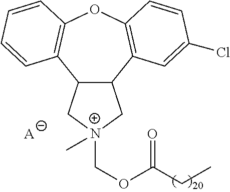 |
| 61 | 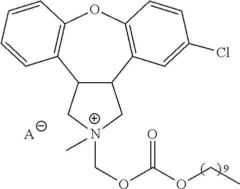 |
| 62 | 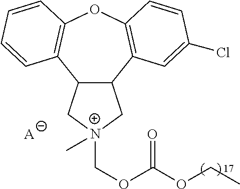 |
| 63 | 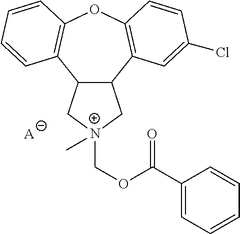 |
| 64 | 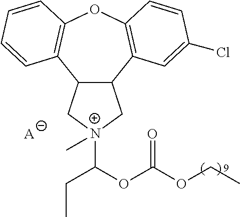 |
| 65 | 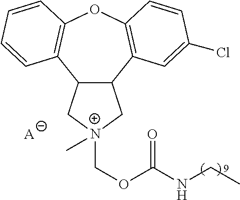 |
| 66 | 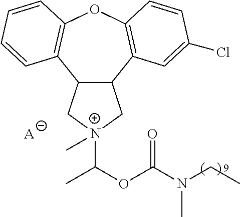 |
| 67 | 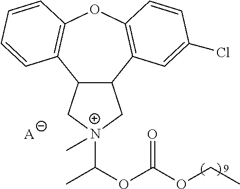 |
| 68 | 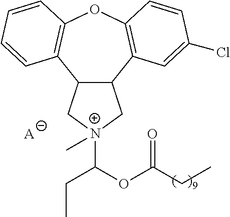 |
| 69 | 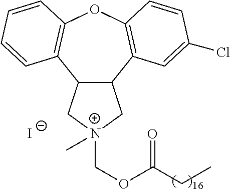 |
| 70 | 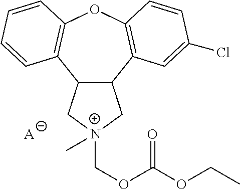 |
| 71 | 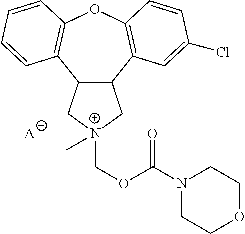 |
| 72 | 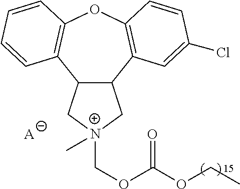 |
| 73 | 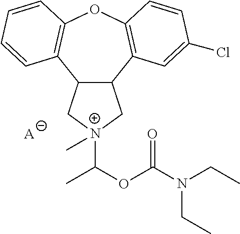 |
| 74 | 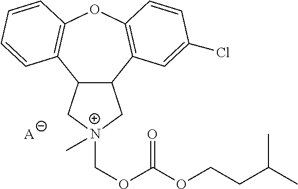 |
| 75 | 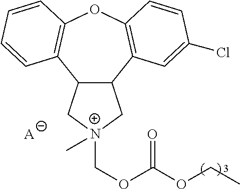 |
| 76 | 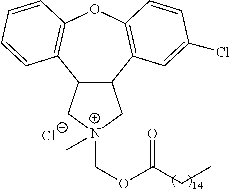 |
| 77 | 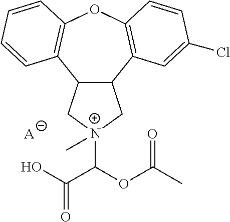 |
| 78 | 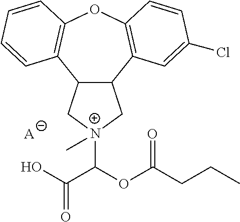 |
| 79 | 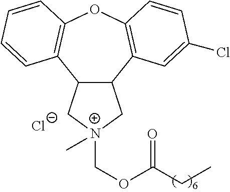 |
| 80 | 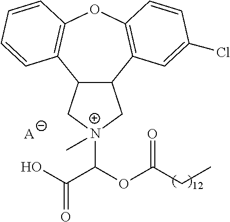 |
| 81 | 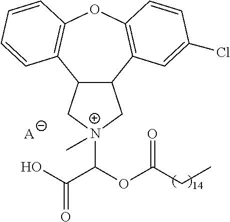 |
| 82 | 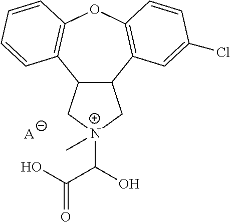 |
| 83 | 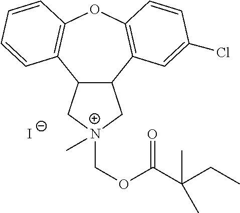 |
| 84 | 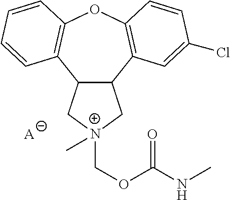 |
| 85 | 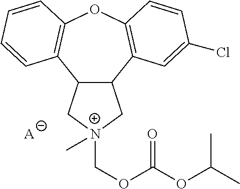 |
| 86 | 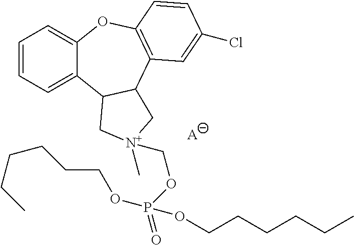 |
| 87 | 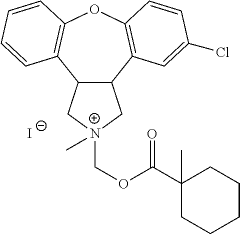 |
| 88 | 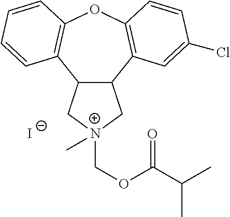 |
| 89 | 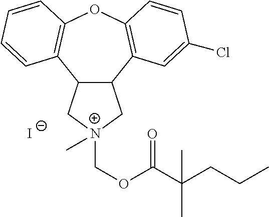 |
| 90 | 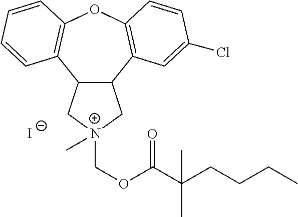 |
| 91 | 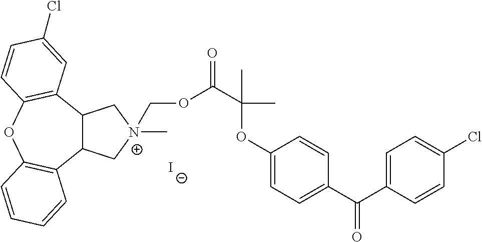 |
| 92 | 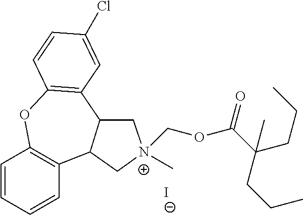 |
| 93 | 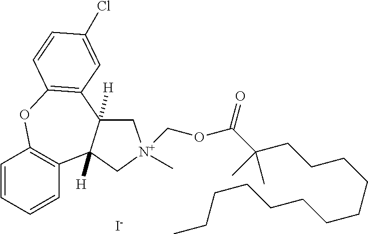 |
| 94 | 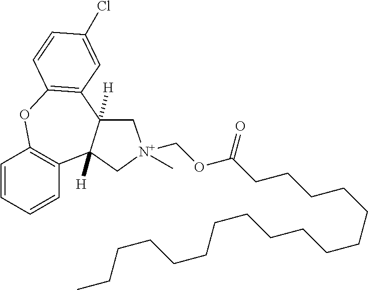 |
| 95 | 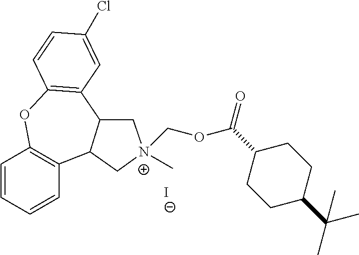 |
| 96 | 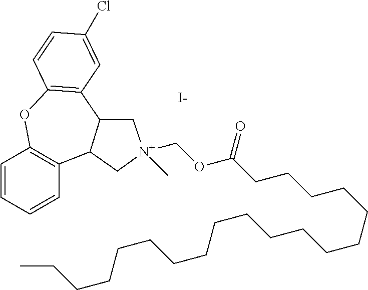 |
| 97 | 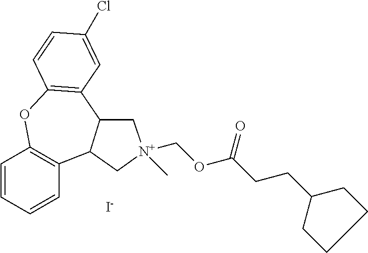 |
| 98 | 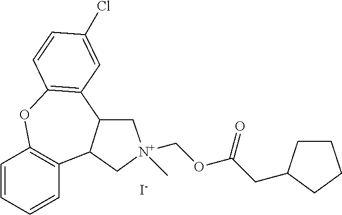 |
| 99 | 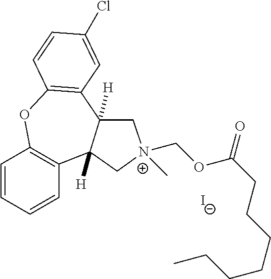 |
| 100 | 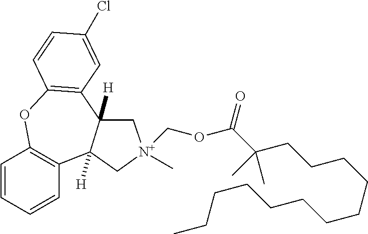 |
| 101 | 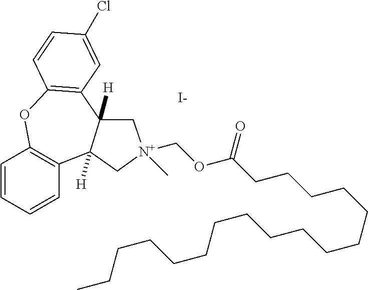 |
| 102 | 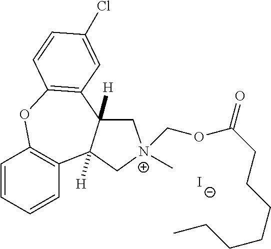 |
| 103 | 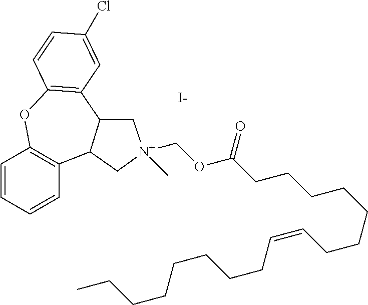 |
| 104 | 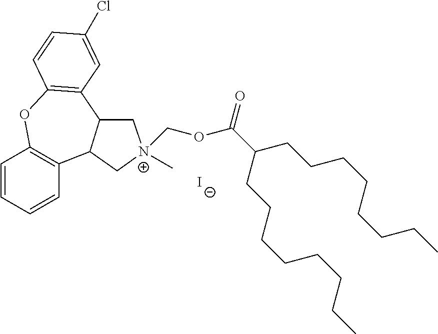 |
| 105 | 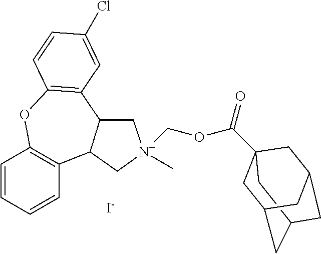 |
| 106 | 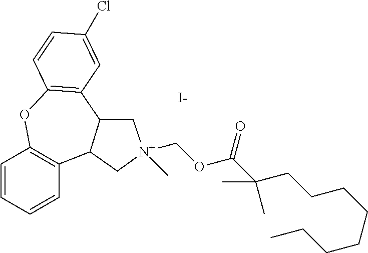 |
| 107 | 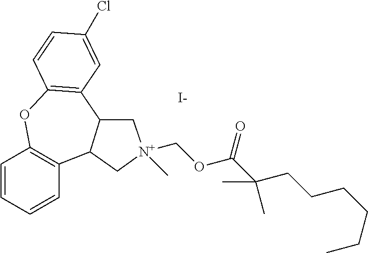 |
| 108 | 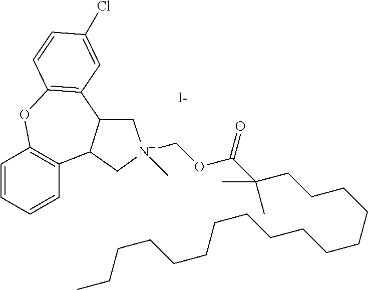 |
| 109 | 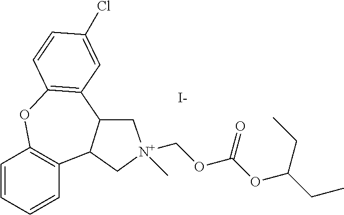 |
| 110 | 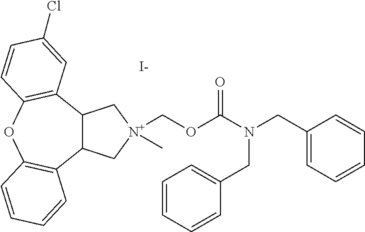 |
| 111 | 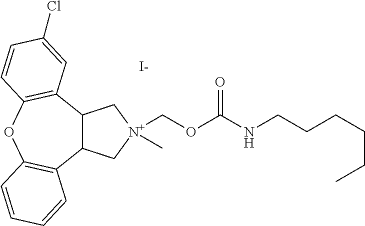 |
| 112 | 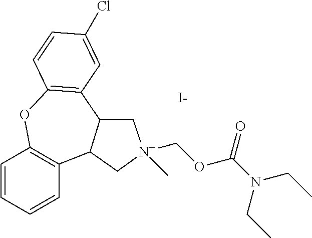 |
| 113 | 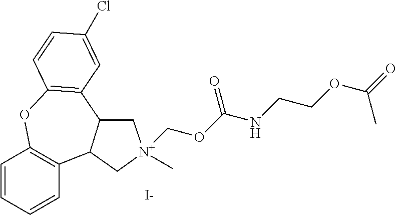 |
| 114 | 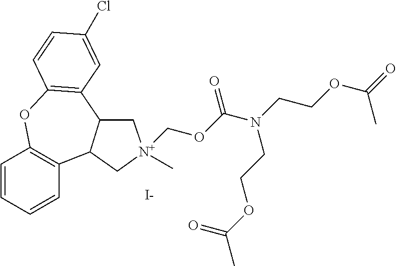 |
| 115 | 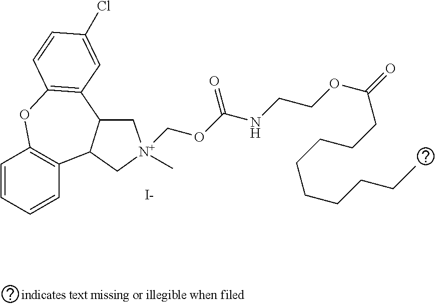 |
| 116 | 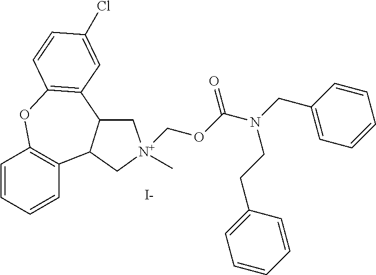 |

In another aspect of the invention, a general method to convert compounds of formula III to substituted tertiary amines is provided (Scheme 1).

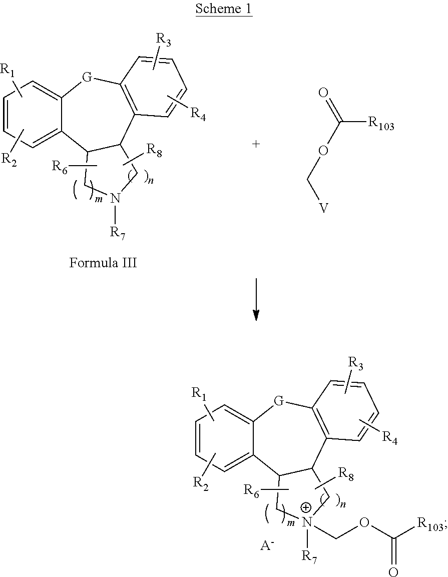

wherein V is a leaving group. A preferred V is selected from —I, —Br, —Cl, tosylate, triflate, mesylate and acetate.

The invention also provides methods for sustained release delivery and methods for pH independent sustained release delivery comprising administering to a patient a therapeutically effective amount of a compound of formula I or formula II.

The invention also provides methods for reducing sedation in a patient as compared to the parent drug comprising administering to a patient a therapeutically effective amount of a compound of formula I or formula II.

### DEFINITIONS

Listed below are definitions of various terms used to describe this invention. These definitions apply to the terms as they are used throughout this specification and claims, unless otherwise limited in specific instances, either individually or as part of a larger group.

The term “acyl” refers to a carbonyl substituted with hydrogen, alkyl, partially saturated or fully saturated cycloalkyl, partially saturated or fully saturated heterocycle, aryl, or heteroaryl. For example, acyl includes groups such as (C₁-C₆) alkanoyl (e.g., formyl, acetyl, propionyl, butyryl, valeryl, caproyl, t-butylacetyl, etc.), (C₃-C₆) cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “alkyl” is intended to include both branched and straight chain, substituted or unsubstituted, saturated aliphatic hydrocarbon radicals/groups having the specified number of carbons. Preferred alkyl groups comprise about 1 to about 24 carbon atoms (“C₁-C₂₄”) preferably about 7 to about 24 carbon atoms (“C₇-C₂₄”), preferably about 8 to about 24 carbon atoms (“C₈-C₂₄”), preferably about 9 to about 24 carbon atoms (“C₉-C₂₄”). Other preferred alkyl groups comprise at about 1 to about 8 carbon atoms (“C₁-C₈”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tert-butyl, n-pentyl, neopentyl and n-hexyl radicals.

The term “alkenyl” refers to linear or branched radicals having at least one carbon-carbon double bond. Such radicals preferably contain from about two to about twenty-four carbon atoms (“C₂-C₂₄”) preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”) preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkynyl radicals are “lower alkynyl” radicals having two to about ten carbon atoms such as propargyl, 1-propynyl, 2-propynyl, 1-butyne, 2-butynyl and 1-pentynyl. Preferred lower alkynyl radicals include 2 to about 6 carbon atoms (“C₂-C₆”).

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkylene,” as used herein, refers to a divalent group derived from a straight chain or branched saturated hydrocarbon chain having the specified number of carbons atoms. Examples of alkylene groups include, but are not limited to, ethylene, propylene, butylene, 3-methyl-pentylene, and 5-ethyl-hexylene.

The term “alkenylene,” as used herein, denotes a divalent group derived from a straight chain or branched hydrocarbon moiety containing the specified number of carbon atoms having at least one carbon-carbon double bond. Alkenylene groups include, but are not limited to, for example, ethenylene, 2-propenylene, 2-butenylene, 1-methyl-2-buten-1-ylene, and the like.

The term “alkynylene,” as used herein, denotes a divalent group derived from a straight chain or branched hydrocarbon moiety containing the specified number of carbon atoms having at least one carbon-carbon triple bond. Representative alkynylene groups include, but are not limited to, for example, propynylene, 1-butynylene, 2-methyl-3-hexynylene, and the like.

The term “alkoxy” refers to linear or branched oxy-containing radicals each having alkyl portions of one to about twenty-four carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “alkoxyalkyl” refers to alkyl radicals having one or more alkoxy radicals attached to the alkyl radical, that is, to form monoalkoxyalkyl and dialkoxyalkyl radicals.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indanyl and biphenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” refers to unsaturated aromatic heterocyclyl radicals. Examples of heteroaryl radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4- thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “heterocycloalkyl” refers to heterocyclo-substituted alkyl radicals. More preferred heterocycloalkyl radicals are “lower heterocycloalkyl” radicals having one to six carbon atoms in the heterocyclo radical.

The term “alkylthio” refers to radicals containing a linear or branched alkyl radical, of one to about ten carbon atoms attached to a divalent sulfur atom. Preferred alkylthio radicals have alkyl radicals of one to about twenty-four carbon atoms or, preferably, one to about twelve carbon atoms. More preferred alkylthio radicals have alkyl radicals which are “lower alkylthio” radicals having one to about ten carbon atoms. Most preferred are alkylthio radicals having lower alkyl radicals of one to about eight carbon atoms. Examples of such lower alkylthio radicals include methylthio, ethylthio, propylthio, butylthio and hexylthio.

The terms “aralkyl” or “arylalkyl” refer to aryl-substituted alkyl radicals such as benzyl, diphenylmethyl, triphenylmethyl, phenylethyl, and diphenylethyl.

The term “aryloxy” refers to aryl radicals attached through an oxygen atom to other radicals.

The terms “aralkoxy” or “arylalkoxy” refer to aralkyl radicals attached through an oxygen atom to other radicals.

The term “aminoalkyl” refers to alkyl radicals substituted with amino radicals.

Preferred aminoalkyl radicals have alkyl radicals having about one to about twenty-four carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term “alkylamino” denotes amino groups which are substituted with one or two alkyl radicals. Preferred alkylamino radicals have alkyl radicals having about one to about twenty carbon atoms or, preferably, one to about twelve carbon atoms. More preferred alkylamino radicals are “lower alkylamino” that have alkyl radicals having one to about ten carbon atoms. Most preferred are alkylamino radicals having lower alkyl radicals having one to about eight carbon atoms. Suitable lower alkylamino may be monosubstituted N-alkylamino or disubstituted N,N-alkylamino, such as N-methylamino, N-ethylamino, N,N-dimethylamino, N,N-diethylamino or the like.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

The terms “halogen” or “halo” as used herein, refers to an atom selected from fluorine, chlorine, bromine and iodine.

The terms “compound” “drug”, and “prodrug” as used herein all include pharmaceutically acceptable salts, solvates, hydrates, polymorphs, enantiomers, diastereoisomers, racemates and the like of the compounds, drugs and prodrugs having the formulas as set forth herein.

Substituents indicated as attached through variable points of attachments can be attached to any available position on the ring structure.

For simplicity, chemical moieties that are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

As used herein, the term “effective amount of the subject compounds,” with respect to the subject method of treatment, refers to an amount of the subject compound which, when delivered as part of desired dose regimen, brings about management of the disease or disorder to clinically acceptable standards.

“Treatment” or “treating” refers to an approach for obtaining beneficial or desired clinical results in a patient. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., metastasis) of disease, preventing occurrence or recurrence of disease, delay or slowing of disease progression, amelioration of the disease state, and remission (whether partial or total).

The neurological and psychiatric disorders include, but are not limited to, disorders such as cerebral deficit subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, social phobia, obsessive compulsive disorder, and post-traumatic stress disorder (PTSD)), mood disorders (including depression, mania, bipolar disorders), circadian rhythm disorders (including jet lag and shift work), trigeminal neuralgia, hearing loss, tinnitus, macular degeneration of the eye, emesis, brain edema, pain (including acute and chronic pain states, severe pain, intractable pain, neuropathic pain, inflammatory pain, and post-traumatic pain), tardive dyskinesia, sleep disorders (including narcolepsy), eating disorders, attention deficit/hyperactivity disorder, and conduct disorder.

The compounds of the invention can be prepared as acid addition salts. Preferably, the acid is a pharmaceutically acceptable acid. Such acids are described in Stahl, P. H. and Wermuth, C. G. (eds.), Handbook of Pharmaceutical Salts: Properties, Selection and Use, Wiley VCH (2008). Pharmaceutically acceptable acids include acetic acid, dichloroacetic acid, adipic acid, alginic acid, L-ascorbic acid, L-aspartic acid, benzenesulfonic acid, 4-acetamidobenzoic acid, benzoic acid, p-bromophenylsulfonic acid; (+)-camphoric acid, (+)-camphor-10-sulfonic acid, capric acid, caproic acid, caprylic acid, carbonic acid, cinnamic acid, cyclamic acid, dodecylsulfuric acid, ethane-1,2-disulfonic acid, ethanesulfonic acid, 2-hydroxyethanesulfonic acid, sulfuric acid, boric acid, citric acid, formic acid, fumaric acid, galactaric acid, gentisic acid, D-glucoheptonic acid, D-gluconic acid, D-glucuronic acid, glutamic acid, glutaric acid, 2-oxoglutaric acid, glycerophosphoric acid, glycolic acid, hippuric acid, hydrochloric acid, hydrobromic acid, hydroiodic acid, isobutyric acid, DL-lactic acid, lactobionic acid, lauric acid, maleic acid, (−)-L-malic acid, malonic acid, DL-mandelic acid, methanesulfonic acid, naphthalene-1,5-disulfonic acid, naphthalene-2-sulfonic acid, 1-hydroxy-2-naphthoic acid, nicotinic acid, nitric acid, oleic acid, orotic acid, oxalic acid, palmitic acid, pamoic acid, phosphoric acid, propionic acid, (−)-L-pyroglutamic acid, salicyclic acid, 4-aminosalicyclic acid, sebacic acid, stearic acid, succininc acid, (+)-L-tartaric acid, thiocyanic acid, p-toluenesulfonic acid, and undecylenic acid.

The term “pharmaceutically acceptable anion” as used herein, refers to the conjugate base of a pharmaceutically acceptable acid. Such anions include the conjugate base of any the acids set forth above. Preferred pharmaceutically acceptable anions include acetate, bromide, camsylate, chloride, formate, fumarate, maleate, mesylate, nitrate, oxalate, phosphate, sulfate, tartrate, thiocyanate and tosylate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The synthesized compounds can be separated from a reaction mixture and further purified by a method such as column chromatography, high pressure liquid chromatography, trituration or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers and/or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; cyclodextrins such as alpha- (α), beta- (β) and gamma- (γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Pharmaceutically acceptable carriers include any and all solvents, dispersion media, coatings, antibacterial and antifungal agents, isotonic and absorption delaying agents, and the like, compatible with pharmaceutical administration, such as sterile pyrogen-free water. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, dimethylacetamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable suspension or emulsion, such as Intralipid®, Liposyn® or Omegaven, or solution in a nontoxic parenterally acceptable diluent or solvent, for example, as a solution in 1,3-butanediol. Intralipid® is an intravenous fat emulsion containing 10-30% soybean oil, 1-10% egg yolk phospholipids, 1-10% glycerin and water. Liposyn® is also an intravenous fat emlusion containing 2-15% safflower oil, 2-15% soybean oil, 0.5-5% egg phosphatides 1-10% glycerin and water. Omegaven® is an emulsion for infusion containing about 5-25% fish oil, 0.5-10% egg phosphatides, 1-10% glycerin and water.

Among the acceptable vehicles and solvents that may be employed are water, Ringer's solution, USP and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide co-polymers. Depending upon the ratio of drug to polymer and the nature of the particular polymer employed, the rate of drug release can be controlled to a desirable extent. Examples of other biodegradable polymers include poly(orthoesters) poly(anhydrides). Depot injectable formulations are also prepared by entrapping the drug in liposomes or microemulsions that are compatible with body tissues.

In one preferred embodiment, the formulation provides a biocompatible sustained release delivery system that is capable of minimizing the exposure of the prodrug to water. This can be accomplished by formulating the prodrug with a sustained release delivery system that is a polymeric matrix capable of minimizing the diffusion of water into the matrix. Suitable polymers comprising the matrix include poly(lactide) (PLA) polymers and the lactide/glycolide (PLGA) co-polymers as described earlier.

Alternatively, the sustained release delivery system may comprise poly-anionic molecules or resins that are suitable for injection or oral delivery. Suitable polyanionic molecules include cyclodextrins and polysulfonates formulated to form a poorly soluble mass that minimizes exposure of the prodrug to water and from which the prodrug slowly leaves.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, all of which are incorporated herein by reference). A discussion of pulmonary delivery of antibiotics is also found in U.S. Pat. No. 6,014,969, incorporated herein by reference.

By a “therapeutically effective amount” of a prodrug compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect).

In accordance with the invention, the therapeutically effective amount of a prodrug of the invention is typically based on the target therapeutic amount of the tertiary-amine containing parent drug. Information regarding dosing and frequency of dosing is readily available for many tertiary amine-containing parent drugs and the target therapeutic amount can be calculated for each prodrug of the invention. In accordance with the invention, the same dose of a prodrug of the invention provides a longer duration of therapeutic effect as compared to the parent drug. Thus if a single dose of the parent drug provides 12 hours of therapeutic effectiveness, a prodrug of that same parent drug in accordance with the invention that provides therapeutic effectiveness for greater than 12 hours will be considered to achieve a “sustained release”.

The precise dose of a prodrug of the invention depends upon several factors including the nature and dose of the parent drug and the chemical characteristics of the prodrug moiety linked to the parent drug. Ultimately, the effective dose and dose frequency of a prodrug of the invention will be decided by the attending physician within the scope of sound medical judgment. The specific therapeutically effective dose level and dose frequency for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims. General methodology for the preparation of asenapine-related compounds can be found in the following publications: U.S. Pat. No. 4,145,434, U.S. Pat. No. 5,763,476, U.S. Pat. No. 4,263,315 and U.S. Pat. No. 5,985,856. Unless otherwise noted the quaternization of racemic Asenapine provides an about 1:1:1:1 mix of the four possible stereoisomers.

### Example 1

### Asenapine

### Synthesis of Compound 69 (ASP stearate iodide) 5-chloro-2-methyl-2-((stearoyloxy)methyl)-2,3,3a,12b-tetrahydro-1H-dibenzo[2,3:6,7]oxepino[4,5-c]pyrrol-2-ium iodide

General Reaction Procedure I

Step a—Formation of Acid Chloride

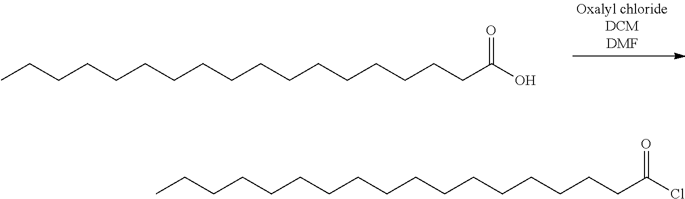

To a stirred suspension of stearic acid (20 g, 70.3 mmol) in dichloromethane (100 mL) was added oxalyl chloride (8.92 mL, 105.5 mmol). One drop dimethylformamide was added and the reaction stirred at 25° C. for 3 hours. The solvent was removed in vacuo and the resulting product used in the next step without further purification.

¹H-NMR (CDCl₃) δ 0.87 (3H, t), 1.20-1.40 (28H, m), 1.65-1.70 (2H, m), 2.87 (2H, t).

### Step B—Formation of Chloromethyl Alkyl Ester

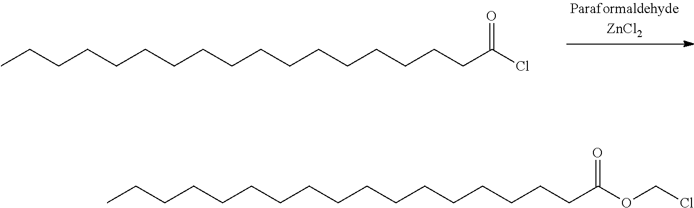

Paraformaldehyde (2.11 g, 70.3 mmol) and zinc chloride (258 mg) were added to the acid chloride prepared above and the reaction mixture was heated at 65° C. for 16 hours and then allowed to cool to 25° C. Dichloromethane (200 mL) and saturated aqueous NaHCO₃ (70 mL) were added. The aqueous emulsion was extracted with dichloromethane (2×50 mL) and the combined organic extracts washed with saturated aqueous NaHCO₃ (70 mL), brine (70 mL), and dried over MgSO₄. After filtration, the volatiles were removed and the residue purified by silica chromatography eluting with heptane to 12% dichloromethane (DCM) in heptane to give a yellow solid (12.64 g, 54% yield over two steps).

¹H-NMR (CDCl₃) δ 0.86 (3H, t), 1.20-1.40 (28H, m), 1.55-1.70 (2H, m), 2.37 (2H, t), 5.70 (2H, s).

### Step C—Formation of Iodomethyl Stearate Ester

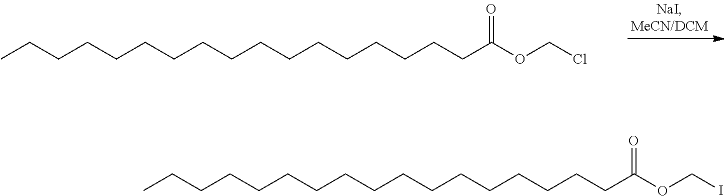

To a solution of the iodomethyl alkyl ester (12.64 g, 37.96 mmol) in acetonitrile (150 mL) and dichloromethane (75 mL) was added sodium iodide (17.07 g, 113.9 mmol). The flask was covered in tin foil to exclude light and stirred at 25° C. for 70 hours and then at 25° C. for 24 hours. The reaction mixture was partitioned between dichloromethane (200 mL) and water (150 mL). The aqueous layer was extracted with dichloromethane (2×150 mL). The combined organics were washed with saturated aqueous (aq) NaHCO₃ (200 mL), 5% aq sodium sulfite solution (200 mL) and brine (2×100 mL), then dried (MgSO₄) and concentrated to give the product as a yellow solid (14.53 g, 90% yield) which was not further purified. ¹H-NMR (CDCl₃) δ 0.87 (3H, t), 1.20-1.35 (28H, m), 1.55-1.70 (2H, m), 2.32 (2H, t), 5.90 (2H, s).

### Step D—Quaternisation Reaction

Asenapine (2 g, 4.85 mmol) and the iodomethyl stearate ester (3.55 g, 14.55 mmol) were stirred together in acetonitrile (50 mL) at 25° C. overnight. The reaction mixture was concentrated and the residue triturated with diethyl ether to give compound 69 (2.80 g, 81% yield).

¹H-NMR (CDCl₃) δ 7.30-7.10 (14H, m), 6.05-5.95 (4H, m), 4.90-4.55 (4H, m), 4.40-3.90 (8H, m), 3.85-3.80 (6H, m), 2.60-2.50 (4H, m), 1.65-1.55 (4H, m), 1.35-1.15 (56H, m), 0.85 (6H, 2×t).

### Synthesis of compound 5 (ASP butyrate chloride) 2-((butyryloxy)methyl)-5-chloro-2-methyl-2,3,3a,12b-tetrahydro-1H-dibenzo[2,3:6,7]oxepino[4,5-c]pyrrol-2-ium chloride

The general procedure I described above was used for the synthesis of compound 5, starting from step B using butyroyl chloride. In step D, 3 equiv of iodomethyl butyrate was used. The iodide salt was converted to the corresponding chloride by passing through Dowex 1X8, 50-100 mesh, ion exchange resin eluting with methanol followed by a diethyl ether trituration then a ethyl acetate trituration to give compound 5 (1.44 g).

¹H-NMR (CDCl₃) δ 7.30-7.00 (14H, m), 6.17-6.11 (4H, m), 4.83-4.72 (2H, m), 4.63-4.53 (2H, m), 4.28-3.97 (7H, m), 3.95-3.83 (7H, m), 2.48 (4H, 2×t), 1.66 (4H, 2×sextet), 0.95 (6H, 2×t).

### Synthesis of compound 47 (ASP laurate chloride) 5-chloro-2-((dodecanoyloxy)methyl)-2-methyl-2,3,3a,12b-tetrahydro-1H-dibenzo[2,3:6,7]oxepino[4,5-c]pyrrol-2-ium chloride

The general procedure I described above was used for the synthesis of compound 47, starting from step B using lauroyl chloride. In step D, 3 equiv of iodomethyl laurate was used. The iodide salt was converted to the corresponding chloride by passing through Dowex 1X8, 50-100 mesh, ion exchange resin eluting with dichloromethane. The exchange was then repeated followed by an diethyl ether trituration to give compound 47 (1.89 g).

¹H-NMR (CDCl₃) δ 7.29-7.09 (14H, m), 6.15-6.10 (4H, m), 4.81-4.73 (2H, m), 4.63-4.57 (2H, m), 4.31-3.83 (14H, m), 2.48 (4H, 2×t), 1.68-1.51 (4H, m), 1.29-1.18 (32H, m), 0.86 (6H, 2×t).

### Synthesis of compound 76 (ASP palmitate chloride) 5-chloro-2-methyl-2-((palmitoyloxy)methyl)-2,3,3a,12b-tetrahydro-1H-dibenzo[2,3:6,7]oxepino[4,5-c]pyrrol-2-ium chloride

The general procedure I described above was used for the synthesis of compound 76, starting from step B using palmitoyl chloride. In step D, 3 equiv of iodomethyl palmitate was used. The iodide salt was converted to the corresponding chloride by passing through Dowex 1X8, 50-100 mesh, ion exchange resin eluting with dichloromethane. The exchange was then repeated followed by an diethyl ether trituration to give compound 76 (2.05 g).

¹H-NMR (CDCl₃) δ 7.26-7.07 (14H, m), 6.17-6.12 (4H, m), 4.83-4.71 (2H, m), 4.64-4.52 (2H, m), 4.27-3.84 (14H, m), 2.49 (4H, 2×t), 1.64-1.58 (4H, m), 1.32-1.16 (48H, m), 0.87 (6H, 2×t).

### Synthesis of compound 9 (ASP pivalate chloride) 5-chloro-2-methyl-2-((pivaloyloxy)methyl)-2,3,3a,12b-tetrahydro-1H-dibenzo[2,3:6,7]oxepino[4,5-c]pyrrol-2-ium chloride

The general procedure I described above was used for the synthesis of compound 9 starting from step C using chloromethyl pivalate. In step D, 3 equiv of iodomethyl pivalate was used. The iodide salt was converted to the corresponding chloride by passing through Dowex 1X8, 50-100 mesh, ion exchange resin eluting with methanol followed by an diethyl ether trituration to provide compound 9 (1.96 g).

¹H-NMR (CDCl₃) δ 7.30-7.05 (14H, m), 6.12-6.10 (4H, m), 4.75-4.55 (4H, m), 4.30-3.90 (8H, m), 3.87-3.85 (6H, m), 1.27 (18H, 2×s).

### Synthesis of compound 79 (ASP octanoate chloride) 5-chloro-2-methyl-2-((octanoyloxy)methyl)-2,3,3a,12b-tetrahydro-1H-dibenzo[2,3:6,7]oxepino[4,5-c]pyrrol-2-ium chloride

The general procedure I described above was used for the synthesis of compound 79 starting from step B using octanoyl chloride. In step D, 3 equiv of iodomethyl octanoate was used. The iodide salt was converted to the corresponding chloride by passing through Dowex 1X8, 50-100 mesh, ion exchange resin eluting with methanol followed by an diethyl ether trituration, to provide compound 79 (1.58 g).

¹H-NMR (CDCl₃) δ 7.30-7.00 (14H, m). 6.20-6.10 (4H, m), 4.85-4.55 (4H, m), 4.40-3.90 (8H, m), 3.90-3.80 (6H, m), 2.55-2.40 (4H, m), 1.70-1.50 (4H, m), 1.35-1.10 (16H, m) 0.85 (6H, 2×t).

### Synthesis of compound 8 (ASP decanoate iodide) 5-chloro-2-((decanoyloxy)methyl)-2-methyl-2,3,3a,12b-tetrahydro-1H-dibenzo[2,3:6,7]oxepino[4,5-c]pyrrol-2-ium iodide

The general procedure I described above was used for the synthesis of compound 8 starting from step B using decanoyl chloride. In step D, 3 equiv of iodomethyl decanoate was used. After diethyl ether trituration compound 8 (3.04 g) was obtained.

¹H-NMR (CDCl₃) δ 7.31-7.10 (14H, m), 6.06-6.00 (4H, m), 4.89-4.76 (2H, m), 4.71-4.58 (2H, m), 4.37-3.83 (14H, m), 2.53 (4H, 2×t), 1.67-1.54 (4H, m), 1.34-1.14 (24H, m), 0.85 (6H, 2×t).

### Synthesis of compound 83 (ASP dimethyl butyrate iodide) 5-chloro-2-4(2,2-dimethylbutanoyl)oxy)methyl)-2-methyl-2,3,3a,12b-tetrahydro-1H-dibenzo[2,3:6,7]oxepino[4,5-c]pyrrol-2-ium iodide

The general procedure I described above was used for the synthesis of compound 83 starting from step B using 2,2-dimethylbutyryl chloride. In step D, 3 equiv of iodomethyl 2,2-dimethylbutyrate was used. After diethyl ether trituration compound 83(2.61 g) was obtained.

¹H-NMR (CDCl₃) δ 7.30-7.10 (14H, m), 6.05-5.95 (4H, m), 4.80-4.60 (4H, m), 4.45-3.95 (8H, m), 3.90-3.80 (6H, m), 1.70-1.60 (4H, m), 1.23 (12H, 2×s), 0.85 (6H, 2×t).

### Synthesis of compound 87 (ASP 2-methyl cyclohexyl carboxylate iodide) 5-chloro-2-methyl-2-(((1-methylcyclohexanecarbonyl)oxy)methyl)-2,3,3a,12b-tetrahydro-1H-dibenzo[2,3:6,7]oxepino[4,5-c]pyrrol-2-ium iodide.

Synthesized using the general procedure I starting from 1-methyl cyclohexane carboxylic acid. After diethyl ether trituration compound 87 (2.75 g) was obtained.

¹H-NMR (300 MHz, CDCl₃) δ 7.32-7.05 (14H, m), 6.00 (2H, s), 5.95 (2H, s), 4.76-4.52 (4H, m), 4.39-3.82 (12H, m), 2.04-2.00 (4H, m), 1.56-1.28 (23H, m).

### Synthesis of compound 88 (ASP isobutyrate iodide) 5-chloro-2-((isobutyryloxy)methyl)-2-methyl-2,3,3a,12b-tetrahydro-1H-dibenzo[2,3:6,7]oxepino[4,5-c]pyrrol-2-ium iodide

Synthesized using the general procedure I starting from isobutyryl chloride. After dissolving in a minimum amount of tetrahydrofuran followed by precipitation with diethyl ether compound 88 (2.23 g) was obtained. ¹H-NMR (300 MHz, CDCl₃) δ 7.30-7.09 (14H, m), 6.03 (2H, s), 5.99 (2H, s), 4.85-4.54 (4H, m), 4.37-3.89 (8H, m), 3.48-3.82 (6H, 2×s), 2.83-2.72 (2H, m), 1.25 (12H, 2×d).

### Synthesis of compound I (ASP Dimethyl myristate iodide) 5-chloro-2-(((2,2-dimethyltetradecanoyl)oxy)methyl)-2-methyl-2,3,3a,12b-tetrahydro-1H-dibenzo[2,3:6,7]oxepino[4,5-c]pyrrol-2-ium iodide

### Synthesis of methyl 2,2-dimethyltetradecanoate

To a stirred solution of diisopropylamine (6.90 mL, 49.0 mmol) in tetrahydrofuran (50 mL) under Ar (g) at −7° C. was added ⁿBuLi (2.3M in hexanes, 21.3 mL, 49.0 mmol) dropwise via a dropping funnel keeping the temperature between 0° C. and 5° C. The reaction was stirred at −7° C. for 30 min and then cooled to −78° C. Methyl isobutyrate (5.61 mL, 49.0 mmol) was added and the reaction stirred at −78° C. for 1.5 hours. 1-Iodododecane (13.05 g, 44.1 mmol) in tetrahydrofuran (10 mL) was added dropwise via a dropping funnel keeping the temperature below −70° C. Further tetrahydrofuran (40 mL) was added over 5 min to aid stirring. After complete addition the reaction was stirred at −78° C. for approximately 2 hours and then allowed to slowly warm to 25° C. overnight.

The reaction was quenched with sat. aq. NH₄Cl (100 mL) and diluted with ethyl acetate (100 mL). The aqueous layer was extracted with ethyl acetate (2×50 mL) and the combined organics washed with brine (50 mL) and dried over MgSO₄. After filtration, the volatiles were removed. The reaction was repeated in a similar manner using methyl isobutyrate (15.05 mL, 131.27 mmol). The two crude batches were combined and purified by silica chromatography eluting heptane to 50% dichloromethane/heptane to give methyl 2,2-dimethyl myristate (31.7 g).

### Synthesis of 2,2-dimethyltetradecanoic acid

To a stirred solution of methyl 2,2-dimethyltetradecanoate (31.7 g, 117.2 mmol) in ethanol (234 mL) was added 2M NaOH (117 mL, 234.4 mmol). The reaction was stirred at 25° C. overnight. NaOH (4.69 g, 117 mmol) was added and the reaction heated at 50° C. for 24 hours. NaOH (4.69 g, 117 mmol) was added and the reaction heated to 100° C. for 4 hours and then cooled to 25° C. 4M HCl (140 mL) was added to acidify. Ethyl acetate (200 mL) was added and the layers separated. The aqueous was extracted with ethyl acetate (2×100 mL) and the combined organics concentrated in vacuo. The residue was partitioned between ethyl acetate (200 mL) and brine (100 mL). The organic layer was washed with brine (50 mL) and dried over MgSO₄. After filtration, the volatiles were removed to give 2,2-dimethyltetradecanoic acid (26.9 g).

Compound 1 was prepared using the general procedure I starting from 2,2-dimethyltetradecanoic acid (synthesized as described above). After diethyl ether trituration compound I (1.07 g) was obtained.

¹H-NMR (300 MHz, CDCl₃) δ 7.32-7.05 (14H, m), 6.02-5.91 (4H, m), 4.78-4.59 (4H, m), 4.44-3.98 (8H, m), 3.92-3.84 (6H, m), 1.62-1.50 (4H, m), 1.34-1.11 (52H, m), 0.88 (6H, 2×t).

### Synthesis of compound 3 (ASP 2-propyl pentanoate iodide) 5-chloro-2-methyl-2-(((2-propylpentanoyl)oxy)methyl)-2,3,3a,12b-tetrahydro-1H-dibenzo[2,3:6,7]oxepino[4,5-c]pyrrol-2-ium iodide

Synthesized using the general procedure I starting from 2,2-di-n-propylacetic acid. After diethyl ether trituration compound 3 (2.46 g) was obtained.

¹H-NMR (300 MHz, CDCl₃) δ 7.33-7.05 (14H, m), 6.04-5.94 (4H, m), 4.78-4.54 (4H, m), 4.43-3.96 (8H, m), 3.93-3.84 (6H, m), 2.62-2.50 (2H, m), 1.72-1.43 (8H, m), 1.38-1.18 (8H, m), 0.93-0.83 (12H, m).

### Synthesis of compound 89 (ASP dimethyl pentanoate iodide) 5-chloro-2-(((2,2-dimethylpentanoyl)oxy)methyl)-2-methyl-2,3,3a,12b-tetrahydro-1H-dibenzo[2,3:6,7]oxepino[4,5-c]pyrrol-2-ium iodide

Synthesized using the general procedure I starting from 2,2-dimethylvaleric acid. After diethyl ether trituration compound 89 (2.58 g) was obtained. ¹H-NMR (300 MHz, CDCl₃) δ 7.30-7.06 (14H, m), 6.02-5.94 (4H, m), 4.77-4.58 (4H, m), 4.41-4.30 (2H, m), 4.25-3.97 (6H, m), 3.90-3.84 (6H, m) 1.59-1.52 (4H, m), 1.29-1.18 (16H, m), 0.87 (6H, 2× t).

### Synthesis of compound 90 (ASP dimethyl hexanoate iodide) 5-chloro-2-(((2,2-dimethylhexanoyl)oxy)methyl)-2-methyl-2,3,3a,12b-tetrahydro-1H-dibenzo[2,3:6,7]oxepino[4,5-c]pyrrol-2-ium iodide

Synthesized in a similar manner to compound I from methyl isobutyrate and 1-iodobutane. After diethyl ether trituration compound 90 (2.50 g) was obtained. ¹H-NMR (300 MHz, CDCl₃) δ 7.32-7.06 (14H, m), 6.03-5.92 (4H, m), 4.78-4.57 (4H, m), 4.44-3.97 (8H, m), 3.94-3.83 (6H, m) 1.62-1.51 (4H, m), 1.34-1.10 (20H, m), 0.84 (6H, 2×t).

### Synthesis of Compound 94—((+)-ASP-stearate iodide) 5-chloro-2-methyl-2-((stearoyloxy)methyl)-2,3,3a,12b-tetrahydro-1H-dibenzo[2,3:6,7]oxepino[4,5-c]pyrrol-2-ium

### Step D—Quaternisation Reaction

This was synthesized employing general reaction procedure I starting from (+)-Asenapine (835 gm, 2.92 mmol) and stearic acid to give Compound 94 (1.98 g, 95%), which was obtained as an approx 1:1 mixture of 2 conformers. ¹H-NMR (300 MHz, CDCl₃) δ 7.31-7.08 (14H, m), 6.04-5.99 (4H, m), 4.84-3.88 (12H, m), 3.83-3.80 (6H, 2×s), 2.56-2.52 (4H, m), 1.71-1.56 (4H, m), 1.37-1.16 (56H, m), 0.88 (6H, 2×t).

### Synthesis of Compound 101—((−)-ASP-stearate iodide) 5-chloro-2-methyl-2-((stearoyloxy)methyl)-2,3,3a,12b-tetrahydro-1H-dibenzo[2,3:6,7]oxepino[4,5-c]pyrrol-2-ium iodide

This was synthesized employing general reaction procedure I starting from stearic acid and (−)-Asenapine. Compound 101 (1.92 g, 91%) was obtained as an approx 1:1 mixture of 2 conformers. ¹H-NMR (300 MHz, CDCl₃) δ 7.31-7.08 (14H, m), 6.05-6.00 (4H, m), 4.86-4.53 (4H, m), 4.39-3.85 (8H, m), 3.84-3.82 (6H, m), 2.57-2.49 (4H, m), 1.64-1.58 (4H, m), 1.31-1.15 (56H, m), 0.87 (6H, 2×t).

### Synthesis of Compound 99—((+)-ASP-octanoate iodide)5-chloro-2-methyl-2-((octanoyloxy)methyl)-2,3,3a,12b-tetrahydro-1H-dibenzo[2,3:6,7]oxepino[4,5-c]pyrrol-2-ium iodide

This was synthesized employing general reaction procedure I starting from octanoyl chloride and (+)-Asenapine to give Compound 99 (1.55 g, 78%) as an approx 1:1 mixture of 2 conformers. ¹H-NMR (300 MHz, CDCl₃) δ 7.32-7.09 (14H, m), 6.04-6.01 (4H, m), 4.87-4.56 (4H, m), 4.38-3.82 (14H, m), 2.55-2.52 (4H, m), 1.76-1.59 (4H, m), 1.36-1.11 (16H, m), 0.85 (6H, 2×t).

### Synthesis of Compound 102—((−)-ASP-octanoate iodide) 5-chloro-2-methyl-2-((octanoyloxy)methyl)-2,3,3a,12b-tetrahydro-1H-dibenzo[2,3:6,7]oxepino[4,5-c]pyrrol-2-ium iodide

This was synthesized employing general reaction procedure I starting from octanoyl chloride and (−)-Asenapine. Compound 102 (1.34 g, 67%) was obtained as an approx 1:1 mixture of 2 conformers. ¹H-NMR (300 MHz, CDCl₃) δ 7.31-7.08 (14H, m), 6.05-5.98 (4H, m), 4.89-4.59 (4H, m), 4.40-3.82 (14H, m), 2.55-2.49 (4H, m), 1.64-1.60 (4H, m), 1.31-1.10 (16H, m), 0.85 (6H, 2×t).

### Synthesis of Compound 95—(ASP trans 4-tBu-cyclobutylcarboxylate iodide) 2-((((1,4-trans)-4-(tert-butyl)cyclohexanecarbonyl)oxy)methyl)-5-chloro-2-methyl-2,3,3a,12b-tetrahydro-1H-dibenzo[2,3:6,7]oxepino[4,5-c]pyrrol-2-ium iodide

General Reaction Procedure II

### Chloromethyl ester of 4-trans-t-butyl cyclohexane carboxylic acid

To a suspension of 4-trans-t-butyl cyclohexane carboxylic acid (5 g, 27.1 mmol) in water (50 mL) was added sodium carbonate (11.5 g, 108.5 mmol). After 20 minutes the reaction mixture was cooled to 0° C. the dichloromethane (100 mL) and chloromethyl chlorosulfate (3.6 mL, 35.3 mmol). The reaction was stirred at 0° C. for 1 hour then allowed to warm to 25° C. and stirred overnight. The reaction mixture was separated and the aqueous washed with dichloromethane (100 mL). The combined organics were dried (MgSO₄) and concentrated to give the crude product which was purified by filtering through silica eluting with 40% dichloromethane/heptane to give the product (4.91 g, 78%).

The product from this was then converted to the corresponding iodide using general reaction procedure I step C and the quaternization reaction was carried out using general reaction procedure I step D to give Compound 95 (2.71 g, 98%). ¹H-NMR (300 MHz, CDCl₃) δ 7.25-7.04 (14H, m), 6.01-5.97 (4H, m), 4.83-4.47 (2H, m), 4.31-4.04 (6H, m), 3.81-3.77 (6H, m), 2.49-2.35 (2H, m), 2.10-2.05 (4H, m), 1.88-1.85 (4H, m), 1.56 (8H, s), 1.51-1.41 (4H, m), 1.11-0.98 (6H, m), 0.84 (18H, s).

### Synthesis of Compound 91—(ASP Fenofibrate iodide) 5-chloro-2-(((2-(4-(4-chlorobenzoyl)phenoxy)-2-methylpropanoyl)oxy)methyl)-2-methyl-2,3,3a,12b-tetrahydro-1H-dibenzo[2,3:6,7]oxepino[4,5-c]pyrrol-2-ium iodide.

This was synthesized employing general reaction procedure II starting from 2-[4-(4-Chlorobenzoyl)-phenoxy]-2-methylpropionic acid. The solvent was removed from the quaternization reaction and the solid triturated with diethyl ether, filtered and dried under vacuum to give Compound 91 (2.03 g, 97%).

¹H-NMR (300 MHz, CDCl₃) δ 7.77-7.61 (8H, m), 7.48-7.38 (4H, m), 7.28-7.10 (8H, m), 7.08-6.78 (10H, m), 6.61-6.36 (4H, m), 4.75-4.59 (2H, m), 4.43-4.29 (2H, m), 4.14-3.94 (8H, m), 3.83-3.67 (6H, m), 1.79-1.72 (12H, m).

### Synthesis of Compound 60—(ASP C22 iodide) 5-chloro-2-((docosanoyloxy)methyl)-2-methyl-2,3,3a,12b-tetrahydro-1H-dibenzo[2,3:6,7]oxepino[4,5-c]pyrrol-2-ium.

This was synthesized employing general reaction procedure II starting from behenic acid to give Compound 60 (3.9 g 94%).

¹H-NMR (300 MHz, CDCl₃) δ 7.30-7.10 (7H, m), 6.02 (2H, d), 4.88-4.55 (2H, m), 4.39-3.88 (4H, m), 3.83 (3H, m), 2.57-2.48 (2H, m), 1.66-1.60 (2H, m), 1.32-1.20 (36H, m), 0.87 (3H, t).

### Synthesis of Compound 97—(ASP cypionate iodide) 5-chloro-2-(((3-cyclopentylpropanoyl)oxy)methyl)-2-methyl-2,3,3a,12b-tetrahydro-1H-dibenzo[2,3:6,7]oxepino[4,5-c]pyrrol-2-ium iodide.

This was synthesized employing general reaction procedure II starting from 3-cyclopentylpropanoic acid to give Compound 97 (0.83 g 84%). ¹H-NMR (300 MHz, CDCl₃) δ 7.30-7.07 (7H, m), 6.05-5.99 (2H, m), 4.88-4.73 (1H, m), 4.70-4.55 (1H, m), 4.40-3.86 (4H, m), 3.83 (3H, m), 2.60-2.59 (2H, m), 1.78-1.42 (9H, m), 1.10-1.00 (2H, m).

### Synthesis of Compound 98—(ASP cyclopentyl acetate iodide) 5-chloro-2-((2-cyclopentylacetoxy)methyl)-2-methyl-2,3,3a,12b-tetrahydro-1H-dibenzo[2,3:6,7]oxepino[4,5-c]pyrrol-2-ium iodide.

This was synthesized employing general reaction procedure II starting from 2-cyclopentylacetic acid to give Compound 98 (0.84 g 87%). ¹H-NMR (300 MHz, CDCl₃) δ 7.31-7.08 (7H, m), 6.03-5.99 (2H, m), 4.86-4.54 (2H, m), 4.38-3.88 (4H, m), 3.83 (2H, m), 2.57-2.50 (2H, m), 2.28-2.16 (1H, m), 1.86-1.76 (2H, m), 1.67-1.44 (4H, m), 1.20-1.05 (2H, m).

### Synthesis of Compound 103—(ASP Oleate iodide) (Z)-5-chloro-2-methyl-2-((oleoyloxy)methyl)-2,3,3a,12b-tetrahydro-1H-dibenzo[2,3:6,7]oxepino[4,5-c]pyrrol-2-ium iodide

This was synthesized employing general reaction procedure II starting from oleic acid to give Compound 103 (1.69 g 49%).

¹H-NMR (300 MHz, CDCl₃) δ 7.29-7.07 (7H, m), 6.02-5.99 (2H, m), 5.38-5.25 (2H, m), 4.85-4.55 (2H, m), 4.37-4.25 (1H, m), 4.22-3.90 (3H, m), 3.83 (3H, m), 2.55-2.49 (2H, m), 2.03-1.95 (4H, m), 1.77-1.67 (2H, m), 1.33-1.20 (20H, m), 0.86 (3H, t).

### Synthesis of Compound 105—(ASP Adamantate iodide) 2-((((1s,3s)-adamantane-1-carbonyl)oxy)methyl)-5-chloro-2-methyl-2,3,3a,12b-tetrahydro-1H-dibenzo[2,3:6,7]oxepino[4,5-c]pyrrol-2-ium iodide.

This was synthesized employing general reaction procedure II starting from adamantane carboxylic acid to give Compound 105 (2.70 g, 85%). ¹H-NMR (300 MHz, CDCl₃) δ 7.30-7.22 (5H, m), 7.22-7.17 (2H, m), 5.44-5.37 (2H, m), 4.50-4.45 (1H, m), 4.33-4.25 (1H, m), 4.25-3.86 (4H, m), 2.02-1.95 (3H, m), 1.95-1.88 (6H, m), 1.73-1.66 (6H, m).

### Synthesis of Compound 6—(ASP Isovalerate iodide) 5-chloro-2-methyl-2-(((3-methylbutanoyl)oxy)methyl)-2,3,3a,12b-tetrahydro-1H-dibenzo[2,3:6,7]oxepino[4,5-c]pyrrol-2-ium iodide

This was synthesized employing general reaction procedure I starting from isovaleryl chloride to give Compound 6 (0.83 g, 92%) was obtained. ¹H-NMR (300 MHz, CDCl₃) δ 7.32-7.09 (14H, m), 6.04-6.01 (4H, m), 4.87-4.56 (4H, m), 4.36-3.91 (8H, m), 3.85-3.83 (6H, 2×s), 2.44-2.42 (4H, m), 2.15-2.04 (2H, m), 0.97 (12H, 2×d).

### Synthesis of Compound 104—(ASP Octyldecanoate iodide) 5-chloro-2-methyl-2-(((2-octyldecanoyl)oxy)methyl)-2,3,3a,12b-tetrahydro-1H-dibenzo[2,3:6,7]oxepino[4,5-c]pyrrol-2-ium iodide

General Reaction Procedure III

### Step A—Synthesis of Diethyl 2,2-dioctylmalonate

To a solution of diethylmalonate (20 g, 0.125 mol) in tetrahydrofuran (500 mL) was added octyl bromide (47 mL, 0.275 mol), followed by sodium hydride (60% in mineral oil, 11 g, 0.275 mol) over 1 h. The reaction mixture was stirred at 25° C. for 3 days. A second portion of sodium hydride (5 g, 0.125 mol) and octyl bromide (15 mL, 0.086) were added and the mixture heated at reflux for 5 hours. The reaction was cooled, carefully quenched with water and then diluted with 2M HCl. The reaction mixture was extracted with ethyl acetate, dried over MgSO₄ and evaporated. The residue was further purified by flash column chromatography eluting with 1:1 heptane/toluene to toluene gave diethyl 2,2-dioctylmalonate (41.4 g, 86%) as a pale yellow oil. ¹H-NMR (300 MHz, CDCl₃) δ 3.98 (4H, q), 1.70-1.60 (4H, m), 1.15-0.88 (30H, m), 0.69 (6H, t).

### Step B—Synthesis of 2-Octyldecanoic acid

To diethyl 2,2-dioctylmalonate (41.4 g, 0.108 mol) was added industrial methylated spirit (50 mL), followed by a solution of KOH (40 g, 0.714 mol) in water (500 mL). The reaction mixture was heated at reflux for 20 hours, poured into ice/water and made acidic with 2M HCl. The mixture was then extracted with ethyl acetate and the organic phase dried over MgSO₄ before evaporation of the volatiles. The residue was then heated neat at 170° C. until gas evolution had ceased (˜5 h) and on cooling 2-octyldecanoic acid (26.4 g, 86%) was obtained as a yellow solid. ¹H-NMR (300 MHz, CDCl₃) δ2.40-2.26 (1H, m), 1.66-1.52 (2H, m), 1.51-1.39 (2H, m), 1.35-1.18 (24H, m), 0.87 (3H, t).

### Step C—Synthesis of Chloromethyl 2-octyldecanoate

To a mixture of 2-octyldecanoic acid (12.2 g, 42.9 mmol) and water (90 mL) was added Na₂CO₃ (17.7 g, 108 mmol), tetrabutylammonium hydrogensulfate (2.8 g, 8.2 mmol), dichloromethane (180 mL) and then chloromethyl chlorosulfate (5.5 mL, 54.3 mmol). The reaction mixture was stirred for 18 h and then diluted with water (300 mL) and dichloromethane (300 mL). The organic phase was separated, dried over MgSO₄ and evaporated. The residue was purified on silica eluting with heptane/dichloromethane (8:1) to give chloromethyl 2-octyldecanoate (12.0 g, 84%) as a colorless oil. ¹H-NMR (300 MHz, CDCl₃) δ 5.72 (2H, s), 2.43-2.33 (1H, m), 1.67-1.52 (2H, m), 1.51-1.40 (2H, m), 1.33-1.18 (24H, m), 0.86 (3H, t).

The product from this was then converted to the corresponding iodide using general reaction procedure I step C and the quaternization reaction was carried out using general reaction procedure I step D to give Compound 104 (3.09 g, 100%). ¹H-NMR (300 MHz, CDCl₃) δ 7.33-7.03 (14H, m), 6.02-5.93 (4H, m), 4.74-4.57 (4H, m), 4.34-4.28 (2H, m), 4.21-3.94 (4H, m), 3.90-3.87 (6H, 2×s), 2.54-2.48 (2H, m), 1.76-1.47 (8H, m), 1.31-1.12 (48H, m), 0.88-0.84 (12H, 2×t).

### Synthesis of Compound 93—((+)-ASP-dimethyl myristate iodide) 5-chloro-2-(((2,2-dimethyltetradecanoyl)oxy)methyl)-2-methyl-2,3,3a,12b-tetrahydro-1H-dibenzo[2,3:6,7]oxepino[4,5-c]pyrrol-2-ium iodide.

To a solution of 2,2-dimethyltetradecanoic acid, synthesized above, (3.5 g, 13.6 mmol) in water (35 mL) was added Na₂CO₃ (5.8 g, 54 mmol). After 20 minutes, the reaction was cooled to 0° C. and nBu₄NHSO₄ (0.93 g, 3 mmol), dichloromethane (75 mL) and chloromethyl chlorosulfate (1.8 mL, 17.7 mmol) was added. The reaction was allowed to warm to 25° C. and stirred overnight. The reaction mixture was separated and the aqueous extracted with dichloromethane (2×100 mL). The combined organics were dried (MgSO₄) and concentrated in vacuo. The product was purified by column chromatography eluting with heptane to 10% dichloromethane/heptane to give the product (5.0 g, 71%). ¹H-NMR (CDCl₃) δ 7.01-6.89 (3H, m), 6.71-6.66 (1H, m), 6.37 (1H, s), 5.77 (2H, s), 5.40 (1H, s), 4.04-3.90 (2H, m), 3.84-3.67 (6H, m), 3.57 (3H, s), 2.31 (3H, s), 1.59-1.49 (2H, m), 1.31-1.10 (26H, m), 0.87 (3H, t).

The product from this was then converted to the corresponding iodide using general reaction procedure I step C and the quaternization reaction was carried out using general reaction procedure I step D with (+)-Asenapine to give Compound 93 (1.93 g, 81%) as an approx 1:1 mixture of 2 conformers. ¹H-NMR (300 MHz, CDCl₃) δ 7.32-7.05 (14H, m), 6.01-5.94 (4H, m), 4.78-4.59 (4H, m), 4.44-3.98 (8H, m), 3.89-3.87 (6H, m), 1.59-1.50 (4H, m), 1.34-1.11 (52H, m), 0.87 (6H, 2×t).

### Compound 100—((−)-ASP-dimethyl myristate iodide) 5-chloro-2-(((2,2-dimethyltetradecanoyl)oxy)methyl)-2-methyl-2,3,3a,12b-tetrahydro-1H-dibenzo[2,3:6,7]oxepino[4,5-c]pyrrol-2-ium iodide.

This was synthesized employing general reaction procedure II starting from 2,2-dimethyltetradecanoic acid and (−)-Asenapine to give Compound 100 (1.97 g, 85%) was obtained as an approx 1:1 mixture of 2 conformers. ¹H-NMR (300 MHz, CDCl₃) δ 7.32-7.07 (14H, m), 6.01-5.94 (4H, m), 4.73-4.58 (4H, m), 4.41-3.96 (8H, m), 3.89-3.86 (6H, m), 1.59-1.56 (4H, m), 1.31-1.11 (52H, m), 0.87 (6H, 2×t).

### Compound 92—(2-Methyl-2propyl pentanoate iodide) 5-chloro-2-methyl-2-(((2-methyl-2-propylpentanoyl)oxy)methyl)-2,3,3a,12b-tetrahydro-1H-dibenzo[2,3:6,7]oxepino[4,5-c]pyrrol-2-ium iodide.

This was synthesized employing general reaction procedure II starting from methyl 2-methylpentanoate to give Compound 92 (1.97 g, 85%). ¹H-NMR (300 MHz, CDCl₃) δ 7.31-7.04 (8H, m), 6.00-5.88 (2H, m), 4.78-4.55 (2H, m), 4.43-4.30 (1H, m), 4.22-4.08 (2H, m), 4.07-3.95 (1H, m), 3.90 (3H, m), 1.68-1.54 (2H, m), 1.53-1.40 (2H, m), 1.38-1.05 (7H, m), 0.90-0.80 (6H, m).

### Compound 59—(Hexyl carbonate iodide) 5-chloro ((((hexyloxy)carbonyl)oxy)methyl)-2-methyl-2,3,3a,12b-tetrahydro-1H-dibenzo[2,3:6,7]oxepino[4,5-c]pyrrol-2-ium iodide

General Reaction Procedures IV

To a solution of chloromethyl chloroformate (9.6 mL, 107.7 mmol) in dichloromethane (100 mL) at 0° C. was added a solution of 1-hexanol (10 g, 97.9 mmol) and pyridine (8.7 mL, 107.7 mmol) in dichloromethane (25 mL) dropwise over 3 hours (keeping the temp at approx 0° C.). The reaction was allowed to gradually warm to 25° C. overnight. 1 M HCl (50 ml) was added to the reaction mixture and separated. The organics were washed with 1M HCl (50 mL), water (100 mL), aq satd NaHCO₃ (2×100 mL), brine (100 mL) and dried (MgSO₄) to give hexyl chloromethyl carbonate (18.53 g, 97%).

The product from this was then converted to the corresponding iodide using general reaction procedure I step C and the quaternization reaction was carried out using general reaction procedure I step D. The quaternization reaction mixture was concentrated and the resulting residue dissolved in a minimum amount of chloroform and diethyl ether was added. A precipitate was formed which was filtered and dried to give Compound 59 (2.09 g, 80%). ¹H-NMR (300 MHz, CDCl₃) δ 7.32-7.04 (14H, m), 6.13-6.04 (4H, m), 4.92-4.54 (4H, m), 4.39-4.03 (8H, m), 3.87-3.84 (6H, 2×s), 1.77-1.59 (8H, m), 1.41-1.18 (12H, m), 0.90-0.86 (6H, 2×t).

### Compound 109—(3-pentanol carbonate) 5-chloro-2-methyl-2-(pentan-3-yloxy)carbonyl)oxy)methyl)-2,3,3a,12b-tetrahydro-1H-dibenzo[2,3:6,7]oxepino[4,5-c]pyrrol-2-ium iodide

This was synthesized employing general reaction procedure IV starting from iodomethyl pentan-3-yl carbonate to give Compound 109 (3.20 g, 91%). ¹H-NMR (300 MHz, CDCl₃) δ 7.32-7.08 (14H, m), 6.06-6.02 (4H, 2×s), 4.87-4.53 (6H, m), 4.41-3.92 (8H, m), 3.88-3.85 (6H, 2×s), 1.72-1.59 (8H, m), 0.92-0.88 (12H, m).

### Compound 112—(Diethyl carbamate) 5-chloro-2-(((diethylcarbamoyl)oxy)methyl)-2-methyl-2,3,3a,12b-tetrahydro-1H-dibenzo[2,3:6,7]oxepino[4,5-c]pyrrol-2-ium iodide

This was synthesized via iodomethyl diethyl carbamate employing general procedure IV. The reaction final mixture concentrated and the resulting residue was dissolved in a minimum amount of dichloromethane and diethyl ether added. A precipitate formed which was filtered then dissolved in dichloromethane and washed with water. The dichloromethane layer was dried and concentrated then triturated with diethyl ether to give a solid which was filtered and dried to give Compound 112 (3.10 g, 91%).

¹H-NMR (300 MHz, CDCl₃) δ 7.31-7.03 (14H, m), 5.93-5.90 (4H, m), 4.82-4.49 (4H, m), 4.38-3.99 (7H, m), 3.97-3.78 (7H, m), 3.40-3.31 (8H, m), 1.24-1.13 (12H, m).

### Compound 110—(Dibenzyl carbamate) 5-chloro-2-(((dibenzylcarbamoyl) oxy)methyl)-2-methyl-2,3,3a,12b-tetrahydro-1H-dibenzo[2,3:6,7]oxepino[4,5-c]pyrrol-2-ium iodide

This was synthesized via iodomethyl dibenzyl carbamate employing general procedure IV. The reaction final mixture was concentrated and the resulting residue dissolved in a minimum amount of chloroform and diethyl ether was added. A precipitate was formed which was filtered and dried to give Compound 110 (3.21 g, >100%). ¹H-NMR (300 MHz, CDCl₃) δ 7.64-6.83 (34H, m), 5.98-5.89 (4H, m), 4.66-3.65 (20H, m), 3.45-3.40 (6H, 2×s).

### Compound 111—(hexyl carbamate) 5-chloro-2-(((hexylcarbamoyl)oxy)methyl)-2-methyl-2,3,3a,12b-tetrahydro-1H-dibenzo[2,3:6,7]oxepino[4,5-c]pyrrol-2-ium iodide.

This was synthesized via iodomethyl hexyl carbamate employing general procedure IV. At the end of the quaternization reaction RDC4560 was filtered off and the mother liqueur concentrated. The residue was triturated with diethyl ether, filtered and the resulting solids then combined to give Compound III (2.58 g, 86%). ¹H-NMR (300 MHz, CDCl₃) δ 7.31-7.05 (7H, m), 6.54 (NH), 5.81 (2H, m), 4.78-4.65 (1H, m), 4.64-4.49 (1H, m), 4.33-4.21 (1H, m), 4.21-4.05 (2H, m), 4.01-3.78 (1H, m), 3.79 (3H, s), 3.18 (2H, q), 1.60-1.49 (2H, m), 1.33-1.19 (6H, m), 0.84 (3H, t).

### Compound 113—(ethanolamine acetate) 2-((((2-acetoxyethyl)carbamoyl)oxy)methyl)-5-chloro-2-methyl-2,3,3a,12b-tetrahydro-1H-dibenzo[2,3:6,7]oxepino[4,5-c]pyrrol-2-ium iodide

This was synthesized employing 2-((iodomethoxy)carbonylamino)ethyl acetate (made using general procedure IV) following general procedure I step D, to give Compound 113 (1.37 g, 91%). ¹H-NMR (300 MHz, DMSO-d₆) δ 7.40-7.25 (5H, m), 5.45-5.35 (2H, m), 4.40-3.85 (8H, m), 3.35-3.25 (2H, m), 1.94 (3H, s).

### Compound 114—(Bis-ethanolamine acetate) 2-(((bis(2-acetoxyethyl) carbamoyl)oxy)methyl)-5-chloro-2-methyl-2,3,3a,12b-tetrahydro-1H-dibenzo[2,3:6,7]oxepino[4,5-c]pyrrol-2-ium iodide.

This was synthesized employing 2,2′-((iodomethoxy)carbonylazanediyl) bis(ethane-2,1-diyl) diacetate (made using general procedure IV) following general procedure I step D, to give Compound 114 (1.63 g, 94%).

¹H-NMR (300 MHz, DMSO-d₆) δ 7.40-7.25 (5H, 7.20-7.16 (2H, m), 5.50-5.38 (2H, m), 4.52-4.45 (1H, m), 4.28-3.85 (10H, m), 3.59-3.66 (2H, m), 3.56-3.50 (2H, m), 1.99 (3H, s), 1.96 (3H, s).

### Compound 116—(Benzyl-phenethyl carbamate) 2-(((benzyl(phenethyl) carbamoyl)oxy)methyl)-5-chloro-2-methyl-2,3,3a,12b-tetrahydro-1H-dibenzo[2,3:6,7]oxepino[4,5-c]pyrrol-2-ium iodide

This was synthesized via iodomethyl benzyl(phenethyl)carbamate employing general procedure IV. The reaction mixture was concentrated and the resulting residue dissolved in a minimum amount of chloroform and diethyl ether was added. A precipitate was formed which was filtered and dried to give Compound 116 (1.79 g, 94%). ¹H-NMR (300 MHz, CDCl₃) δ 7.34-6.79 (34H, m), 5.92-5.68 (4H, m), 4.62-3.39 (26H, m), 2.96-2.79 (4H, m).

### Compound 115—(O-decyl ethanolamine carbamate) 5-chloro-2-((((2-(decanoyloxy)ethyl)carbamoyl)oxy)methyl)-2-methyl-2,3,3a,12b-tetrahydro-1H-dibenzo[2,3:6,7]oxepino[4,5-c]pyrrol-2-ium iodide.

This was synthesized via 2-((iodomethoxy)carbonylamino)ethyl decanoate employing general procedure IV. At the end of the quaternization reaction diethyl ether was added to aid precipitation. Filtration and drying gave Compound 115 (0.78 g, 64%).

¹H-NMR (300 MHz, CDCl₃) δ 7.32-7.10 (7H, m), 7.00-6.92 (1H, m), 5.85 (2H, s), 4.80-4.69 (1H, m), 4.65-4.49 (1H, m), 4.32-4.07 (5H, m), 3.97-3.82 (1H, m), 3.78 (3H, s), 3.50-3.42 (3H, m), 2.31 (2H, t), 1.65-1.49 (2H, m), 1.33-1.16 (12H, m), 0.87 (3H, t).

### Example 2

### Solution Stability of Asenapine Prodrugs as a Function of pH

The asenapine derived prodrugs were prepared at approximately 300 ug/mL in buffers (see table of buffers below). The initial ratio of prodrug/parent was measured using a freshly prepared solution in unbuffered water. Acetonitrile was titrated into all samples as needed to ensure the complete dissolution of the compounds. The amount of acetonitrile varied depending on the solubility of each compound (see Note 1). 1.5 mL of each stability sample was transferred into a HPLC vial and the vials were maintained at 25° C. in the temperature controlled sample compartment of the HPLC. Each sample was assayed by HPLC after 1, 4, 10, and 24 hours for prodrug and asenapine content (see Note 2).

The fraction of prodrug remaining at each time point was calculated as;

Fraction Prodrug=(HPLC Area of Prodrug)/(HPLC area of prodrug+asenapine) (see Note 3).

The loss of prodrug was then fit to the equation for first order decay:

Fraction Prodrug=(Initial Fraction prodrug)*e^{-kt}

where t=time (in hours) and k is the rate constant for decay.
Finally, the half-lives are were calculated as

t_{1/2}=0.693/k

Table of Buffers:

| | | Citric | | | |
|---|---|---|---|---|---|
| Buffering | H₃PO₄/ | Acid/Sodium | NaH₂PO₄/ | NaH₂PO₄/ | Glycine/ |
| Agents | NaH₂PO₄ | Citrate | Na₂HPO₄/ | Na₂HPO₄/ | NaOH |
| pH | 2.11 | 5.08 | 5.95 | 6.95 | 9.01 |
| (measured) | | | | | |

All buffers were 0.01M.

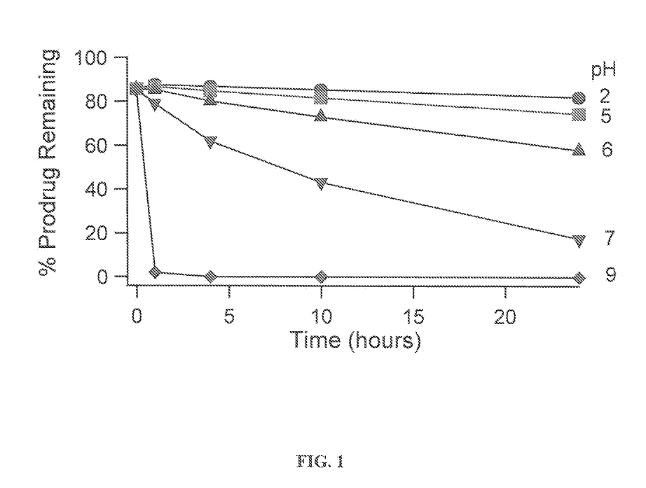

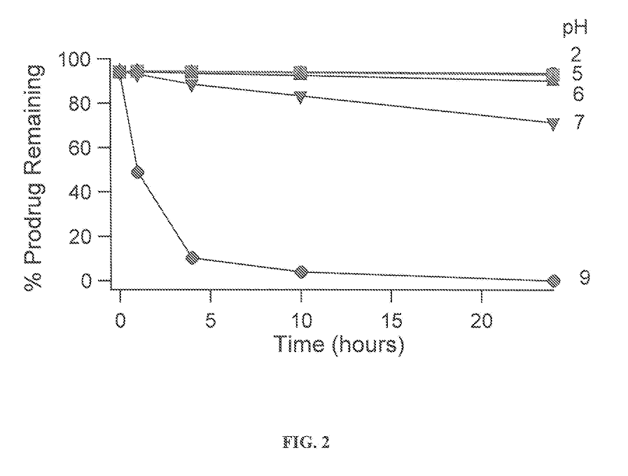

The results are shown in FIG's. 1 and 2. As shown in the figures, asenapine pivalate (FIG. 2) is more stable than the asenapine octanoate (FIG. 1).

Note 1: The acetonitrile concentration is not expected to have a large impact on the degradation rate since the rate follows first order decay with respect to compound (ie, the rate constant is independent of the concentration). The absolute concentration of prodrug does not need to be known since the data are fit as a fraction of prodrug relative to total prodrug + asenapine.

Note 2: A duplicate sample of the pivalate prodrug of asenapine at pH 7 was injected at more frequent time points (initial +0.5, 1, 2, 4, 8, 12, and 24 hours) to ensure that the 5-point curve (includes initial time point +1, 4, 10, and 24 hours)) adequately represents the degradation rate; the two curves were virtually identical.

Note 3: Since HPLC area percents without conversion factors are used in the calculation instead of actual concentration values, therefore, the “Fraction Prodrug” is an estimate and the reported half-lives are estimated based on the area under the curve. However, the trends/conclusion for degradation vs. pH are indisputable. The rank-order of stability for two different prodrugs will also be correct though the relative rates of degradation between any two compounds may differ from those predicted here.

### Example 3

### Pharmacokinetic Evaluation of Asenapine and Asenapine Prodrugs in Rats

Animals: 18 Male Sprague-Dawley rats (Charles River Laboratories, Wilmington, Mass.) were used in the study. Three groups of 6 rats were used and are referred to in this study as Groups A, B and C. Rats were approximately 350-375 g at time of arrival. Rats are housed 2 per cage with ad libitum chow and water. Environmental conditions in the housing room: 64-67° F., 30% to 70% relative humidity, and 12:12-h light:dark cycle. All experiments were approved by the institutional animal care and use committee.

Test Compounds: The following formulations of Asenapine parent drug and prodrug compounds of the invention were used in the study.

| Study | | Dose | Dose volume | Dosing |
|---|---|---|---|---|
| Group | Formulation | mg/rat | (mL)/route | Vehicle |
| A | Asenapine: | 10 | 0.3/IM | 1% HPMC in PBS |
| | Maleic Acid | | | saline with 0.2% |
| | (1:1 molar | | | Tween pH 6.0 |
| | ratio) | | | |
| B | Asenapine | 10 | 0.3/IM | 1% HPMC in PBS |
| | Palmitate | | | saline with 0.2% |
| | Chloride (Cpd | | | Tween pH 6.0 |
| | ASN-76) | | | |
| C | Asenapine | 10 | 0.3/IM | 1% HPMC in PBS |
| | Dimethyl | | | saline with 0.2% |
| | butyrate | | | Tween pH 6.0 |
| | Iodide (Cpd | | | |
| | ASN-83) | | | |

Pharmacokinetics study: Rats were dosed IM by means of a 23 gauge, 1 in. needle with 1 cc syringe 0.3 mL suspension was withdrawn from the vial containing the test compound. The rat was injected in the muscles of the hind limb after anesthesia with isoflourane. Blood samples were collected via a lateral tail vein after brief anesthesia with Isoflurane. A 27½G needle and 1 cc syringe without an anticoagulant was used for the blood collection. Approximately 350 μL of whole blood was collected at each sampling time point of 6 hours, 24 hours and 2, 5, 7, 9, 12, 14, 21, 28, 35 days after administration. Once collected, whole blood was immediately transferred to tubes containing K₂ EDTA, inverted 10-15 times and immediately placed on ice. The tubes were centrifuged for 2 minutes at >14,000 g′ s (11500 RPMs using Eppendorf Centrifuge 5417C, F45-30-11 rotor) at room temperature to separate plasma. Plasma samples were transferred to labeled plain tubes (MICROTAINER®; MFG# BD5962) and stored frozen at <−70° C.

Data Analysis: Drug concentrations in plasma samples were analyzed by liquid chromatography—mass spectroscopy using appropriate parameters for each compound. Half-life, volume of distribution, clearance, maximal concentration, and AUC were calculated by using WinNonlin version 5.2 software (Pharsight, St. Louis, Mo.).

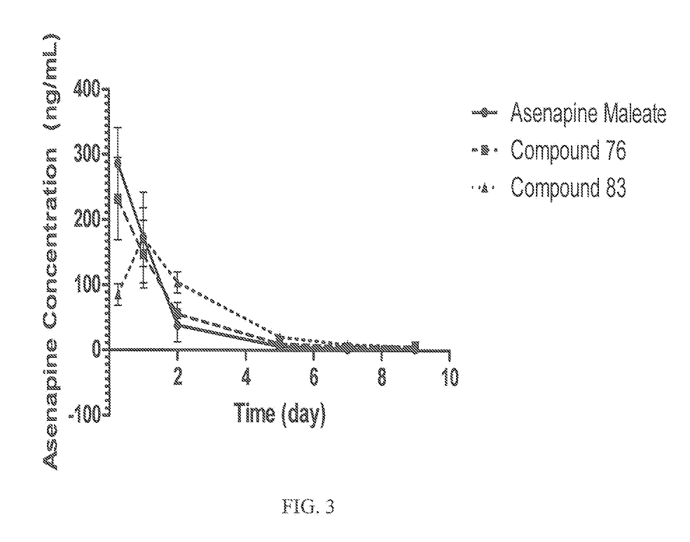

Results: The results are shown in FIG. 3. As is seen in FIG. 3, the Cmax of, the Asenapine dimethyl butyrate prodrug (ASN-83), was lower than the Cmax of the parent Asenapine formulation as well as lower than that of the Asenapine palmitate prodrug (ASN-76). It should be noted that the first sampled time point on the graph is 6 hours and therefore it is likely that the Cmax for Asenapine and Asenapine palmitate was earlier than 6 hours. The Asenapine dimethyl butyrate prodrug had a longer duration of action and a more gradual decrease in plasma concentration over all the time points sampled as compared to either Asenapine, or Asenapine palmitate prodrug.

It appears that the Asenapine palmitate prodrug formulation may have undergone rapid hydrolytic cleavage likely due to exposure to bulk water in the injection formulation. This prodrug will be tested in combination with a sustained delivery system that minimizes exposure of the prodrug to bulk water.

### Example 4

### Pharmacodynamic Studies Using an Amphetamine-Induced Locomotion Model

Introduction: Prodrugs of the invention useful in the treatment of schizophrenia and bipolar disorder are expected to show predictive validity in rodent models of hyperlocomotion. D-Amphetamine-induced locomotion is postulated to mimic the dopaminergic hyperactivity which forms the basis for the “dopamine hypothesis” of schizophrenia. The AMPH-induced hyperactivity model provides a simple, initial screen of antipsychotic compound efficacy. See, Fell et al., Journal of Pharmacology and Experimental Therapeutics, (2008) 326:209-217. Amphetamine induced hyperactivity is used to screen various doses of prodrug formulations of asenapine to measure pharmacodynamic efficacy in an acute hyperlocomotion paradigm. The hypothesis of the study is that PO administration of asenapine prodrug formulations, which result in plasma concentrations of ˜100-200 ng/ml, will produce a significant attenuation of AMPH-induced locomotion.

General behavior and activity can be measured in experimental animals (typically rats and mice) in order to assess psychomotor stimulant properties, anxiogenic/anxiolytic or sedative properties of a drug. As such, open-field studies can provide insight into the behavioral effects of test compounds. Certain prodrugs of the present invention are useful in the treatment of schizophrenia and bipolar disorder. Asenapine is a parent tertiary-amine containing drug from which prodrugs of the invention are derived that is useful in the treatment of schizophrenia and bipolar disorder. Such asenapine prodrugs of the invention show predictive validity in rodent models of hyperlocomotion. D-Amphetamine-induced locomotion is postulated to mimic the dopaminergic hyperactivity which forms the basis for the “dopamine hypothesis” of schizophrenia. Likewise, glutamate NMDA receptor antagonist (MK-801, PCP, etc.) induced locomotion is postulated to mimic the NMDA hypoactivity hypothesis of schizophrenia (Fell et al., supra). These tests of drug-induced hyperactivity provide simple, initial screens of antipsychotic compound efficacy. Amphetamine induced hyperactivity will be used to screen various prodrugs of asenapine, administered in oil solutions, to measure pharmacodynamic efficacy. The results of the D-AMPH induced locomotion done in this study will be compared to the historical results of subcutaneous (S.C.) asenapine administration on D-AMPH. The hypothesis of the study is that exposure to asenapine prodrugs, which results in asenapine concentrations that are efficacious at locomotor testing, will display efficacy in in vivo measures of antipsychotic efficacy.

Materials: Experimental animals: 12, Sprague Dawley rats are purchased from Charles River Laboratory. The rats are approximately 90 days old, and weighed in the range of 350-275 grams upon receipt from the supplier. One rat is placed in each cage and allowed to acclimate for about 1 week. The rats are provided with food and water ad libitum.

Dosing solution of D-Amphetamine (D-AMPH): D-AMPH is purchased from Sigma Aldrich. D-amphetamine HCl is prepared in 0.9% saline to a concentration of 1.5 mg/ml. D-Amphetamine was given I.P. per body weight at a dose of 1 ml/kg (=1.5 mg/kg). Salt form correction is not used in accordance with historical literature. D-Amphetamine is prepared fresh from solid form 30 min. prior to each test period.

Dosing solutions of prodrug derivatives of antipsychotic parent drugs: Dosing solutions of asenapine prodrugs of the invention useful in the treatment of schizophrenia and biopolar disorder are prepared. Dosing solutions comprise any number of suitable excipients for injection including but not limited to, i) oil emulsion in water with any combination of diphosphotidylcholine (DPPC), glycerol and NaOH, ii) aqueous suspensions including crystalline suspensions in any combination of hydroxypropylmethyl cellulose (HPMC) glycerol, phosphate buffered saline (PBS) and polysorbate (e.g. Tween 20).

Behavior Box: The behavior chambers are purchased from Med Associates, Inc. of St. Albans, Vt., Model ENV-515. Software for measuring animal movement is provided with the behavior chamber by the supplier.

Methods: The animals are acclimated for one week prior to commencing experimentation. The animals are initially acclimated to the behavior box for about 15 minutes before they are removed from the box and injected with 1.5 ml of an asenapine prodrug compound of the invention, at concentrations which produce target therapeutic levels for asenapine approximately 1 hour after administration. After an additional 15 minutes the animals are placed back in the behavior box for an additional 30 minute drug-baseline test session. The mice are then administered by IP injection, D-AMPH (1.5 mg/kg) followed by a 60 minute experimental behavioral measurement period. The parameters that are measured include a) total distance measured (primary measure), b) total number of ambulatory moves (second measure), c) total number of vertical moves (secondary measure) and d) time spent immobile (secondary measure.

Blood Sampling: Tail vein blood is taken on experiment days immediately following locomotor activity measurements (2-hours post-prodrug administration) and again the following day at time-points corresponding to 22 hours post-prodrug administration. Blood samples are collected via a lateral tail vein after anesthesia with Isoflurane. A 27½ G syringe without an anticoagulant is used for the blood collection, and the whole blood is transferred to pre-chilled (wet ice) tubes containing K2 EDTA. 0.5 ml of blood per animal is collected per time point. The tubes are inverted 15-20 times and immediately returned to the wet ice until being centrifuged for 2 minutes ≧14,000 g to separate plasma. The plasma samples that are prepared in this manner are transferred to labeled plain tubes (MICROTAINER®; MFG# BD5962) and stored frozen at <−70° C.

Behavioral Data Acquisition: Behavioral data is captured electronically by the software package associated with the behavior chambers. Data is transformed and analyzed via GraphPad Prism® 5 software (GraphPad Software, Inc., La Jolla, Calif.). The data is analyzed using a 2-way repeated measures ANOVA.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A compound represented by Formula I: 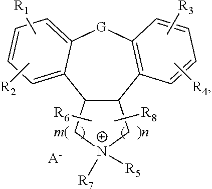
Formula I
or its geometric isomers, enantiomers, diastereomers, racemates, pharmaceutically acceptable salts and solvates thereof;
wherein each m and n is independently selected from 0, 1, 2 or 3;
each R₁, R₂, R₃, R₄, R₆, R₇, and R₈ is independently selected from absent, hydrogen, hydroxy, halogen, —OR₁₀, —SR₁₀, —N(R₁₀)(R₁₁)—, optionally substituted aliphatic, optionally substituted aryl or optionally substituted heterocyclyl;
wherein each R₁₀ and R₁₁ are independently hydrogen, halogen, aliphatic, substituted aliphatic, aryl or substituted aryl;
alternatively, two of R₁, R₂, R₃ and R₄ together form an optionally substituted ring;
R₅ is selected from —C(R₁₀) (R₁₁)—OR₁₂, —C(R₁₀)(R₁₁)—OC(O)OR₂₁, —C(R₁₀)(R₁₁)—OC(O)R₂₁, —C(R₁₀)(R₁₁)—OC(O)NR₁₂R₂₁, —C(R₁₀)(R₁₁)—OPO₃²⁻MY, —C(R₁₀)(R₁₁)—OP(O)(O⁻M)(O R₂₁), —C(R₁₀)(R₁₁)—OP(O)(OR₂₁)(OR₂₂);
each R₁₂ is independently hydrogen, halogen, aliphatic, substituted aliphatic, aryl or substituted aryl;
each R₂₁ and R₂₂ is independently hydrogen, aliphatic, substituted aliphatic, aryl or substituted aryl;
G is selected from —O—, —S—, —NR₁₀, or —C(R₁₀)(R₁)—;
Y and M are the same or different and each is a monovalent cation;
or M and Y together is a divalent cation;
A- is a pharmaceutically acceptable counterion; and
optionally, the prodrug further comprises a biocompatible delivery system for delivering the prodrug wherein the system is capable of minimizing accelerated hydrolytic cleavage of the prodrug by minimizing exposure of the prodrug to water.

2. A compound according to claim 1 represented by Formula II: 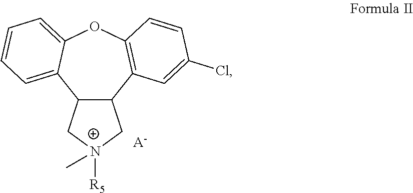
wherein R₅ and A⁻ are as defined in claim 1 and optionally, the prodrug further comprises a biocompatible delivery system for delivering the prodrug wherein the system is capable of minimizing accelerated hydrolytic cleavage of the prodrug by minimizing exposure of the prodrug to water.

3. A compound of claim 1, wherein R₅ is selected from: 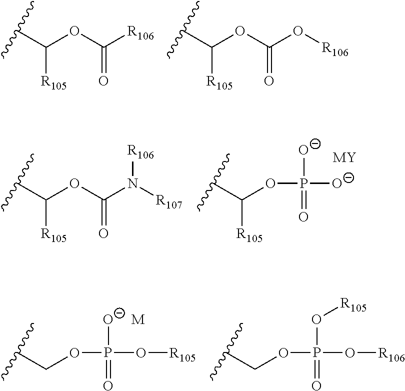
wherein R₁₀₅, R₁₀₆ and R₁₀₇ are independently selected from hydrogen, halogen, optionally substituted C₁-C₂₄ alkyl, optionally substituted C₂-C₂₄ alkenyl, optionally substituted C₂-C₂₄ alkynyl, optionally substituted C₃-C₂₄ cycloalkyl, optionally substituted C₁-C₂₄ alkoxy, optionally substituted C₁-C₂₄ alkylamino and optionally substituted C₁-C₂₄ aryl.

4. A compound of claim 1, wherein R₅ is selected from: 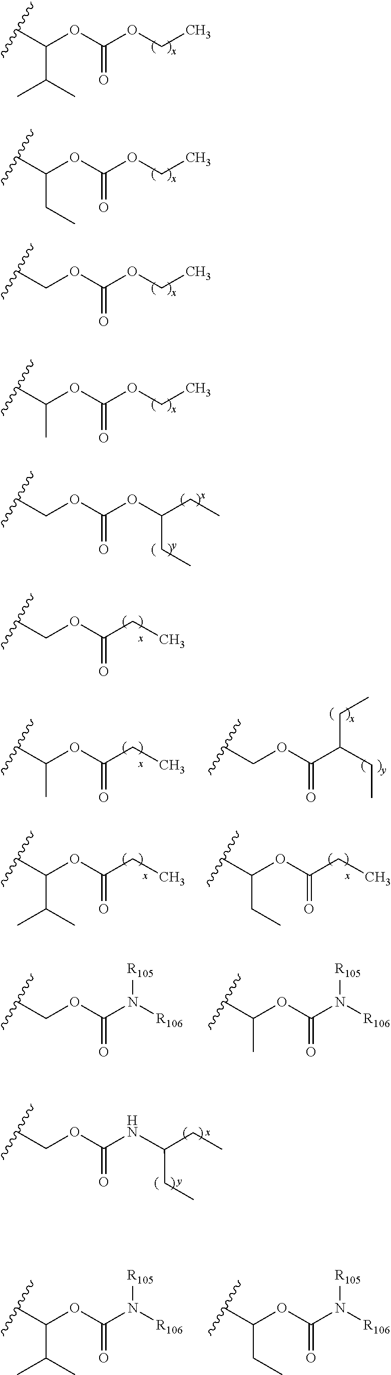 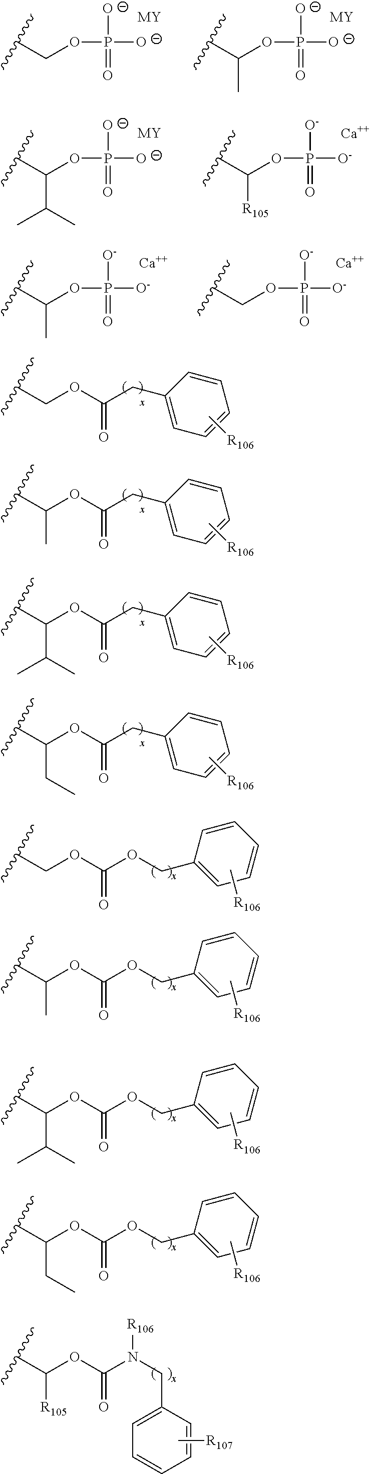 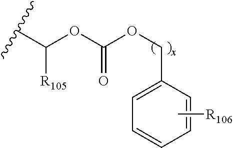
wherein x is an integer between 0 and 30; and
R₁₀₅, R₁₀₆, and R₁₀₇ are as defined above.

5. A compound of claim 1, wherein R₅ is selected from: 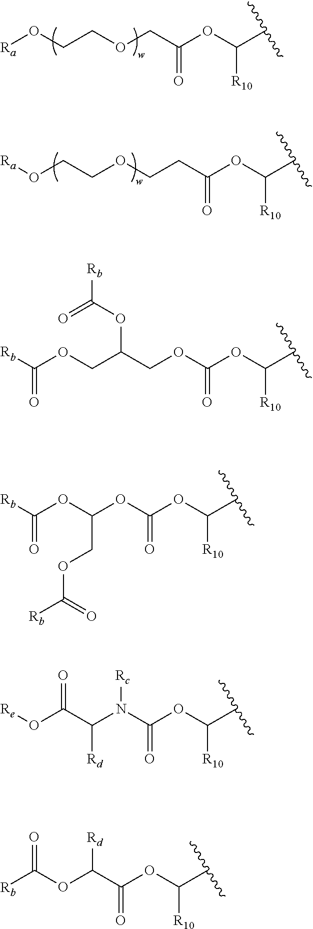
wherein w is 1 to about 1000; each Rₐ, R_{b} and Rₑ is independently C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, aryl or substituted aryl; R_{c} is H or substituted or unsubstituted C₁-C₆-alkyl; R_{d} is H, substituted or unsubstituted C₁-C₆-alkyl, substituted or unsubstituted aryl-C₁-C₆-alkyl or substituted or unsubstituted heteroaryl-C₁-C₆-alkyl; and R₁₀ is as defined above; alternatively R_{c} and R_{d} together with the carbon and nitrogen atoms to which they are attached, forms a heterocycloalkyl group.

6. A compound of claim 1, wherein R₅ is selected from Table 1, 2, 3, 4 or 5.

7. A compound of claim 1 selected from Table A.

8. A compound of claim 1 selected from: 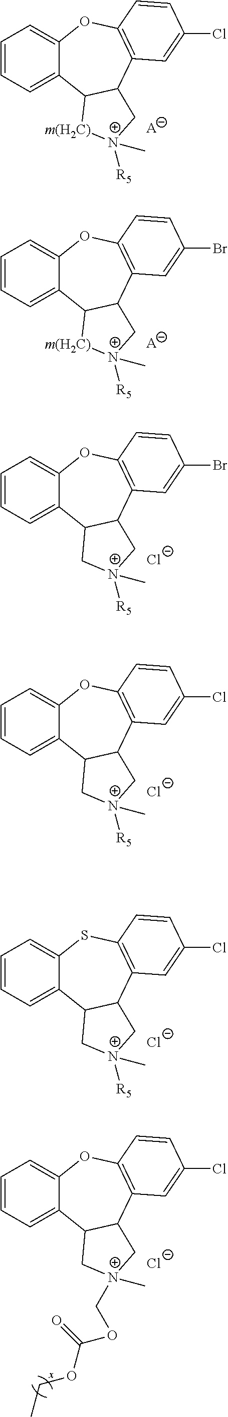 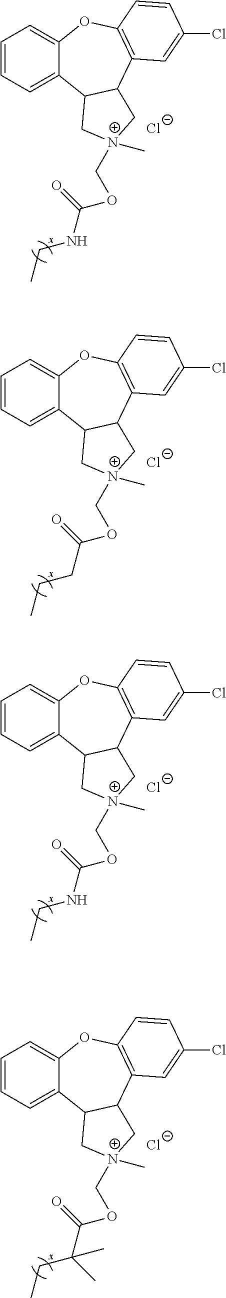 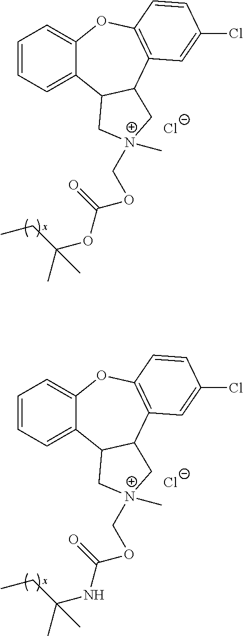
wherein m, R₅ and A⁻ are as defined in claim 1;
x is an integer between 0 and 30.

9. A compound according to claim 1, wherein the relative stereochemistry between the two pyrrolidine ring fusion centers is trans.

10. A method of treating a neurological or psychiatric disorder by administering a compound according to claim 1 to a patient in need thereof.

11. A method according to claim 10, wherein said disorder is schizophrenia.

12. A method according to claim 10, wherein said disorder bipolar I disorder.

13. A method for the synthesis of a compound of formula I: 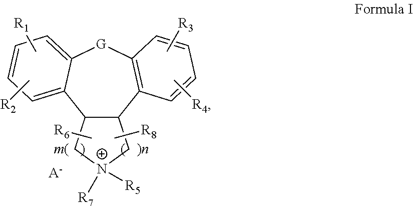
comprising the step of reacting a compound of formula III, 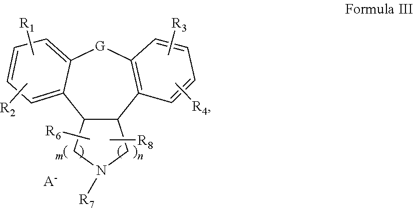
with a compound of the formula R₅—V;
wherein V is a leaving group; and
R₁, R₂, R₃, R₄, R₅, R₆, R₇, G, m, n, and A⁻ are as defined above.

14. The process according to claim 13, wherein V is iodo, bromo, or chloro, and said reaction is performed in an aprotic solvent.

15. A method of pH-independent sustained release delivery of asenapine to a patient comprising administering to the patient a compound according to claim 1.

16. A method of reducing sedation in a patient comprising administering to the patient a compound according to claim 1.
